SEQUENCE LISTING (64 sequences)

SEQ ID NO: 1 - protein, 106 aa, synthetic construct
QSPASLAVSLGQRATISCRASESVDSFGNSFMHWYQQKAGQPPKSSIYRAANLESGIPARFSGSGSRTDFTLTINPVEADDVATYFCQQSYEDPWTFGGGTKLGNQ

SEQ ID NO: 2 - protein, 105 aa, synthetic construct
LVKPGGSLKLSCVASGFTFSSYAMSWVRQTPEKRLEWVASISSGGTTYYPDNVKGRFTISRDNARNILYLQMSSLRSEDTAMYYCARGYYDYHYWGQGTTLTVSS

SEQ ID NO: 3 - protein, 106 aa, synthetic construct
QSPASLAVSLGQRATISCRASESVDSYVNSFLHWYQQKPGQPPKLLIYRASNLQSGIPARFSGSGSRTDFTLTINPVEADDVATYCCQQSNEDPTTFGGGTKLEIK

SEQ ID NO: 4 - protein, 112 aa, synthetic construct
LQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSA

SEQ ID NO: 5 - protein, 101 aa, synthetic construct
QTPSSLSASLGDRVTISCRASQDIKNYLNWYQQPGGTVKVLIYYTSRLHSGVPSRFSGSGSGTDYSLTISNLEQEDFATYFCQRGDTLPWTFGGGTKLEIK

SEQ ID NO: 6 - protein, 116 aa, synthetic construct
LESGPGLVAPSQSLSITCTVSGFSLTNYDISWIRQPPGKGLEWLGVIWTSGGTNYNSAFMSRLSITKDNSESQVFLKMNGLQTDDTGIYYCVRGDGNFYLYNFDYWGQGTTLTVSS

SEQ ID NO: 7 - protein, 15 aa, synthetic construct
RASESVDSFGNSFMH

SEQ ID NO: 8 - protein, 7 aa, synthetic construct
RAANLES

SEQ ID NO: 9 - protein, 9 aa, synthetic construct
QQSYEDPWT

SEQ ID NO: 10 - protein, 5 aa, synthetic construct
SYAMS

SEQ ID NO: 11 - protein, 16 aa, synthetic construct
SISSGGTTYYPDNVKG

SEQ ID NO: 12 - protein, 7 aa, synthetic construct
GYYDYHY

SEQ ID NO: 13 - protein, 15 aa, synthetic construct
RASESVDSYVNSFLH

SEQ ID NO: 14 - protein, 7 aa, synthetic construct
RASNLQS

SEQ ID NO: 15 - protein, 9 aa, synthetic construct
QQSNEDPTT

SEQ ID NO: 16 - protein, 6 aa, synthetic construct
SDSAWN

SEQ ID NO: 17 - protein, 16 aa, synthetic construct
YISYSGSTSYNPSLKS

SEQ ID NO: 18 - protein, 6 aa, synthetic construct
GLRFAY

SEQ ID NO: 19 - protein, 11 aa, synthetic construct
RASQDIKNYLN

SEQ ID NO: 20 - protein, 7 aa, synthetic construct
YTSRLHS

SEQ ID NO: 21 - protein, 9 aa, synthetic construct
QRGDTLPWT

SEQ ID NO: 22 - protein, 5 aa, synthetic construct
NYDIS

SEQ ID NO: 23 - protein, 16 aa, synthetic construct
VIWTSGGTNYNSAFMS

SEQ ID NO: 24 - protein, 12 aa, synthetic construct
GDGNFYLYNFDY

SEQ ID NO: 25 - protein, 238 aa, synthetic construct
METDTILLWVLLLWVPGSTGDIVLTQSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 460 aa, synthetic construct
MKVLSLLYLLTAIPGILSDVQLQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 9 aa, synthetic construct
QQSNEDPYT

SEQ ID NO: 28 - protein, 213 aa, synthetic construct
QSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 439 aa, synthetic construct
LQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 111 aa, synthetic construct
DIVLTQSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIK

SEQ ID NO: 31 - protein, 115 aa, synthetic construct
DVQLQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSS

SEQ ID NO: 32 - protein, 218 aa, synthetic construct
DIVLTQSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 442 aa, synthetic construct
DVQLQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 10 aa, synthetic construct
QNVGSNVDWY

SEQ ID NO: 35 - protein, 6 aa, synthetic construct
KASNRY

SEQ ID NO: 36 - protein, 9 aa, synthetic construct
MQSNTNPPW

SEQ ID NO: 37 - protein, 5 aa, synthetic construct
DYAMN

SEQ ID NO: 38 - protein, 15 aa, synthetic construct
WINTQTGKPTYADDF

SEQ ID NO: 39 - protein, 16 aa, synthetic construct
TYFCTRSTFYYSSYIY

SEQ ID NO: 40 - protein, 11 aa, synthetic construct
KASQNVGSNVD

SEQ ID NO: 41 - protein, 7 aa, synthetic construct
KASNRYT

SEQ ID NO: 42 - protein, 10 aa, synthetic construct
MQSNTNPPWT

SEQ ID NO: 43 - protein, 17 aa, synthetic construct
WINTQTGKPTYADDFKQ

SEQ ID NO: 44 - protein, 16 aa, synthetic construct
STFYYSSYIYGWYFDF

SEQ ID NO: 45 - protein, 15 aa, synthetic construct
RASESVDSYGNSFMH

SEQ ID NO: 46 - protein, 7 aa, synthetic construct
LASNLES

SEQ ID NO: 47 - protein, 9 aa, synthetic construct
QQNNGDPYT

SEQ ID NO: 48 - protein, 5 aa, synthetic construct
TYALN

SEQ ID NO: 49 - protein, 19 aa, synthetic construct
RIRSKSNNYTTYYADSVKD

SEQ ID NO: 50 - protein, 9 aa, synthetic construct
PQSGTSFAY

SEQ ID NO: 51 - protein, 11 aa, synthetic construct
KASQYVGTNVA

SEQ ID NO: 52 - protein, 7 aa, synthetic construct
SASYRCS

SEQ ID NO: 53 - protein, 8 aa, synthetic construct
QQYNTYVT

SEQ ID NO: 54 - protein, 5 aa, synthetic construct
TYWMN

SEQ ID NO: 55 - protein, 17 aa, synthetic construct
RIDPYDSETHYNQNFKD

SEQ ID NO: 56 - protein, 12 aa, synthetic construct
KEGRQWGAYFDY

SEQ ID NO: 57 - protein, 110 aa, synthetic construct
YIVMTQTPTSISISVGERVTMNCKASQNVGSNVDWYQQKTGQSPKLLIYKASNRYTGVPDRFTGSGSGTDFTFTISNMQAVDLAVYYCMQSNTNPPWTFGGGTKLELKRA

SEQ ID NO: 58 - protein, 125 aa, synthetic construct
EVKLLESGGGLVQPSQTLSISCKASGYTFTDYAMNWVKQAPGDGLKWMGWINTQTGKPTYADDFKQRFVFSLETSASTAYLQINNLNIEDTATYFCTRSTFYYSSYIYGWYFDFWGPGTMVTVSS

SEQ ID NO: 59 - protein, 110 aa, synthetic construct
DIVMTQSPTSISISVGERVTMNCKASQNVGSNVDWYQQKTGQSPKLLIYKASNRYTGVPDRFTGSGSGTDFTFTISNMQAVDLAVYYCMQSNTNPPWTFGGGTKLELKRA

SEQ ID NO: 60 - protein, 125 aa, synthetic construct
QIQLVQSGPELKKPGESVKISCKASGYTFTDYAMNWVKQAPGNGLKWMGWINTQTGKPTYADDFKQRFVFSLETSASTAYLQINNLNIEDTATYFCTRSTFYYSSYIYGWYFDFWGPGTMVTVSS

SEQ ID NO: 61 - protein, 112 aa, synthetic construct
NIVLTQSPASLAVSLGQRATISCRASESVDSYGNSFMHWYQQKPGQPPKLLIYLASNLESGVPARFSGSGSRTDFTLTIDPVEADDVATYYCQQNNGDPYTFGGGTKLEIIR

SEQ ID NO: 62 - protein, 120 aa, synthetic construct
EVQLVESGGGLMQPKGSLKLSCAASGFTFKTYALNWVRQAPGTGLEWVARIRSKSNNYTTYYADSVKDRFTISRDDSQNMLYLQMNNLKTEDTAMYYCVRPQSGTSFAYWGQGTLVTVSA

SEQ ID NO: 63 - protein, 107 aa, synthetic construct
DIVMTQSQKFMSTSVGDRVSVTCKASQYVGTNVAWYQQKPGQSPKALIQSASYRCSGVPDRFTGSGSGTDFTLTISNVQSEDLADYFCQQYNTYVTFGGGTKLELKR

SEQ ID NO: 64 - protein, 121 aa, synthetic construct
QVRLQQPGAELVRPGASVKLSCKASGYTFTTYWMNWVKQRPEDGLEWIGRIDPYDSETHYNQNFKDKAILTVDKSSSTAYMQLSSLTYEDSAVYYCTRKEGRQWGAYFDYWGQGTTLTVSS

(19) United States
(12) Patent Application Publication
(10) Pub. No.: US 2026/0242502 A1
(43) Pub. Date: Aug. 20, 2026

(54) AGENTS AND METHODS FOR TREATING MYOPATHIES

(71) Applicants: Implicit Bioscience Limited, Woolloongabba (AU); Belth Israel Deaconess Medical Center. Inc., Boston, MA (US)

(72) Inventors: Kory Joshua Lavine, St. Louis, MO (US); Jeffrey E. Saffiz, Waban, MA (US)

(21) Appl. No.: 19/122,121

(22) PCT Filed: Oct. 18, 2023
(86) PCT No.: PCT/AU2023/051027

§ 371 Date: Apr. 17, 2025

Related U.S. Application Data
(60) Provisional application No. 63/417,313, filed on Oct. 18, 2022.
(60) Provisional application No. 63/464,048, filed on May 4, 2023.

(51) Int. Cl. C07K 16/28; A61K 39/00; A61P 9/00

(52) U.S. Cl. CPC C07K 16/2896; A61P 9/00; A61K 2039/505; C07K 2317/76

(57) ABSTRACT

This disclosure relates generally to methods and agents for treating cardiomyopathies. More particularly, the present disclosure relates to the use of CD14 antagonist antigen-binding molecules for treating a cardiomyopathy, including alleviating or inhibiting the development of adverse cardiac remodeling, improving cardiac function including improving ventricular function and atrial function.

### RELATED APPLICATIONS

This application claims priority to U.S. Provisional Patent Application No. 63/417,313 entitled “Agents and methods for treating myopathies” filed 18 Oct. 2022 and U.S. Provisional Patent Application No. 63/464,048 entitled “Agents and methods for treating myopathies” filed 4 May 2023, the contents of which are incorporated herein by reference in their entirety.

### FIELD

This disclosure relates generally to methods and agents for treating cardiomyopathies. More particularly, the present disclosure relates to the use of CD14 antagonist antigen-binding molecules for treating a cardiomyopathy, including alleviating or inhibiting the development of adverse cardiac remodeling, improving cardiac function including improving ventricular function and atrial function.

### BACKGROUND

Cardiovascular diseases, including hypertension, coronary artery disease and cardiomyopathies may result in heart failure, which is associated with pathological remodeling of the myocardium, pump failure and sudden death. Epidemiological analysis in Western countries indicates that cardiovascular disorders are among the first causes of morbidity and mortality among people over 60 years. There are approximately 600,000 deaths per year in Europe from myocardial infarction and, even more relevant, heart failure is estimated to affect over 15 million people worldwide, representing one of the leading causes of death. This number is likely going to increase as a consequence of the ageing of the global population. While conventional pharmacological treatment strategies (e.g., β-adrenergic blocking agents and angiotensin converting enzyme (ACE) inhibitors) have shown effectiveness in prolonging survival of heart failure patients, the prognosis of affected individuals remains poor, leaving a need for new concepts.

Cardiomyopathies constitute a heterogeneous group of diseases of the myocardium associated with mechanical and/or electrical dysfunction, which cause cardiac dysfunction with heart failure, arrhythmia, and sudden death. These diseases usually (but not invariably) manifest in inappropriate ventricular hypertrophy or dilatation, as well as decreased contractile and/or diastolic function. As compared to the acute phase of myocardial infarction, in which a large number of cardiomyocytes are rapidly necrotized by ischemia and an acute inflammatory reaction ensues, cardiomyopathies do not generally cause such phenomena and are characterized by chronic inflammation and slow progression of structural abnormalities such as cardiomegaly, cardiomyocyte hypertrophy, and myocardial fibrosis.

Cardiomyopathies are typically divided into two major groups, primary cardiomyopathies and secondary cardiomyopathies based on predominant organ involvement. Primary cardiomyopathies (genetic, non-genetic, acquired) are those solely or predominantly confined to heart muscle, and include dilated cardiomyopathy, hypertrophic cardiomyopathy, restrictive cardiomyopathy, arrhythmogenic cardiomyopathy and unclassifiable cardiomyopathy. Secondary cardiomyopathies are those resulting from an underlying condition affecting many areas of the body, including nutritional disorders, metabolic disorders (hyperthyroidism, acromegaly), infiltrative processes (neoplasia, amyloidosis) and inflammatory processes (toxins, immune reactions, infectious agents).

Current drug treatments available for management of cardiomyopathies include vasodilators to reduce blood pressure and ease the workload of the heart, diuretics to reduce fluid overload, inhibitors and blocking agents of the body's neuro-hormonal responses (e.g., ACE inhibitors and β-adrenergic blocking agents), antiarrhythmic drugs, calcium channel blockers, anticoagulants, anti-inflammatoires such as corticosteroids and other medicaments. Such medications, while effective for a short time, often cannot be used for extended periods because of side effects. Various surgical procedures such as heart transplantation have also been proposed for patients who suffer from hypertrophic cardiomyopathy and develop refractory heart failure and/or intractable arrhythmia. Alternatively, an implantable medical device such as ventricular assist devices (VADs) may be implanted in the chest to increase the pumping action of the heart, or an intra-aortic balloon pump (IABP) may be used for maintaining heart function for short periods of time, but typically no longer than one month. While each of these approaches has proven to be at least partly beneficial to patients, they each have shortcomings which limit their overall effectiveness. For example, drug therapies often involve unwanted side effects and complex therapy regimens which contribute to poor patient compliance. Additionally, both drug therapy and surgical approaches are very costly, adding to the health care costs associated with heart failure. Despite the ongoing research and development of treatments for cardiomyopathy, there is still a significant need for improved and alternative treatments.

### SUMMARY

The present disclosure is based in part on the unexpected determination that targeting Cluster of Differentiation 14 (CD14) through administration of a CD14 antagonist antibody can significantly inhibit the development of ventricular dysfunction, adverse cardiac remodeling and fibrosis in a mouse model of arrhythmogenic cardiomyopathy (ACM). In particular, it has been found that administration of a CD14 antagonist antibody to ACM animals at disease onset restores left ventricular ejection fraction (% LVEF) to a level seen in wild-type animals, as shown in FIG. 2, inhibits right ventricular dysfunction by normalizing right ventricular fractional area change (% RVFAC), as shown in FIG. 3, reduces ectopic heart beats by decreasing the frequency of premature ventricular contractions (PVCs), as shown in FIG. 4, restores left ventricular mass (LVM) to a level seen in wild-type animals with associated decrease or minimization of hypertrophic remodeling, as shown in FIG. 5, and reduces myocardial fibrosis, as shown in FIG. 6, which, in turn, reduces myocardial injury. It has also been found that administration of a CD14 antagonist antibody to ACM animals with established disease mitigates significantly against further deterioration of % LVEF, as shown in FIGS. 7 and 8, and myocardial fibrosis, as shown in FIG. 9. Given that in cardiomyopathies CD14 is expressed not only on pro-inflammatory M1 macrophages, but is also expressed on anti-inflammatory M2 macrophages that are involved in healing and tissue repair, these findings are indeed surprising as targeting M2 macrophages with an anti-CD14 antagonist antibody would have been expected to interfere with tissue remodeling and repair, which would likely lead to non-treatment or worsening of disease.

It has also been found that the hearts of ACM animals contain pro-inflammatory CCR2+CD14+ monocytes and macrophages as shown in FIGS. 10B and 10C, which are likely associated with the development of cardiomyopathy disease, and that such pro-inflammatory monocytes and macrophages are also present in the myocardium of cardiomyopathic subjects generally, including cardiac sarcoidosis subjects, as shown for example in FIGS. 13A and 13B. Notably, treatment of ACM animals with an anti-CD14 antagonist antibody at disease onset markedly reduced CCR2+CD14+ macrophages in the hearts of treated animals, as shown in FIGS. 11 and 12.

Based on these findings, the present inventors propose that CD14 antagonists generally, including CD14 antagonist antigen-binding molecules, are useful for treating or alleviating at least one symptom of a cardiomyopathy, for treating or inhibiting development of a cardiomyopathy, for alleviating or inhibiting development of adverse cardiac remodeling in a cardiomyopathy, for improving cardiac function in a cardiomyopathy, for improving ventricular function in a cardiomyopathy, for improving atrial function in a cardiomyopathy, for reducing the number of premature ventricular contractions (PVCs), for treating or alleviating at least one symptom of atrial fibrillation, and/or for reducing the number of premature atrial contractions (PACs), as described for example hereafter.

Accordingly, in one aspect, the present disclosure provides methods of treating or alleviating at least one symptom of a cardiomyopathy in a subject. These methods generally comprise, consist or consist essentially of administering to the subject a CD14 antagonist antigen-binding molecule. The at least one symptom may be selected from ventricular tachycardia, implantable cardioverter-defibrillator (ICD) shock, lung congestion, fluid retention, fatigue, heart murmurs, rapid heartbeat, arrhythmias, chest pain, light-headedness, fainting, dyspnea, peripheral edema, abdominal distention, myocardial fibro-fatty infiltration, embolization, syncope, angina, exercise intolerance, orthopnea, heart failure and sudden cardiac death (SCD). Suitably, the administration of the CD14 antagonist antigen-binding molecule improves one or more clinical parameters of the subject, illustrative examples of which include, reduced left ventricular ejection fraction, left ventricular hypertrophy, increased myocardial fibrosis, adverse cardiac remodeling, right ventricular dysfunction, aberrant heart geometry (e.g., increased right ventricular fractional area change), increased end-diastolic volume, increased frequency of premature ventricular contractions, and increased left ventricular mass.

Disclosed herein in another aspect are methods of alleviating or inhibiting development of adverse cardiac remodeling in a subject with a cardiomyopathy. These methods generally comprise, consist or consist essentially of administering to the subject a CD14 antagonist antigen-binding molecule.

Yet another aspect of the present disclosure provides methods of improving cardiac function in a subject with a cardiomyopathy. These methods generally comprise, consist or consist essentially of administering to the subject a CD14 antagonist antigen-binding molecule. The improvement of cardiac function may comprise any one or more of improving left ventricular function, improving fractional shortening, improving ejection fraction, reducing end-diastolic volume, decreasing left ventricular mass, decreasing arrhythmias, reducing frequency of heart murmurs, reducing heart rate, normalizing of heart geometry, or a combination thereof.

Disclosed herein in still another aspect are methods of improving ventricular function in a subject with a cardiomyopathy. These methods generally comprise, consist or consist essentially of administering to the subject a CD14 antagonist antigen-binding molecule. The administration of a CD14 antagonist antigen-binding molecule suitably improves a measure or metric of ventricular function in the subject, representative examples of which include ventricular strain, systolic function or diastolic function. In some embodiments, the ventricular function may be measured by echocardiography (echo), ambulatory rhythm (Holter) monitoring, cardiac computed tomography (CT), or cardiac magnetic resonance imaging (MRI).

In a further aspect, disclosed herein are methods of improving atrial function in a subject with a cardiomyopathy. These methods generally comprise, consist or consist essentially of administering to the subject a CD14 antagonist antigen-binding molecule. The administration of a CD14 antagonist antigen-binding molecule suitably improves a measure or metric of atrial function in the subject, representative examples of which include atrial reservoir function, atrial conduit function and atrial pump function. In some embodiments, the atrial function may be measured by echocardiography (echo), cardiac MRI, ambulatory rhythm (Holter) monitoring, or cardiac CT.

The cardiomyopathy may be a primary cardiomyopathy or a secondary cardiomyopathy. Representative primary cardiomyopathies include genetic cardiomyopathies, mixed (genetic and non-genetic) cardiomyopathies, and acquired cardiomyopathies. Non-limiting examples of secondary cardiomyopathies include infiltrative cardiomyopathies, storage cardiomyopathies, toxicity cardiomyopathies, granulomatous cardiomyopathies, cardiofacial cardiomyopathies; neuromuscular cardiomyopathies nutritional deficiency cardiomyopathies, autoimmune or collagen cardiomyopathies, and electrolyte imbalance cardiomyopathies. In specific embodiments, the cardiomyopathy is ACM, which is suitably arrhythmogenic right ventricular cardiomyopathy (ARVC). In representative examples of this type, the subject may have one or more mutations in a gene associated with ACM. In some embodiments, the cardiomyopathy is other than ACM (a “non-ACM cardiomyopathy”).

Disclosed herein in yet another aspect are methods of reducing the number of premature ventricular contractions (PVCs) in a subject, wherein the subject is suitably a cardiomyopathy subject. These methods generally comprise, consist or consist essentially of administering to the subject a CD14 antagonist antigen-binding molecule.

Another aspect of the present disclosure provides methods of treating or alleviating at least one symptom of atrial fibrillation in a subject, wherein the subject is suitably a cardiomyopathy subject. These methods generally comprise, consist or consist essentially of administering to the subject a CD14 antagonist antigen-binding molecule. The symptom(s) of atrial fibrillation may be rapid heartbeat, arrhythmias, chest pain, light-headedness, fainting, dyspnea, heart failure, stroke or death. In specific embodiments, the subject has atrial fibrillation.

Yet another aspect of the present disclosure provides methods of reducing the number of premature atrial contractions (PACs) in a subject, wherein the subject is suitably a cardiomyopathy subject. These methods generally comprise, consist or consist essentially of administering to the subject a CD14 antagonist antigen-binding molecule.

In any of the aspects and embodiments disclosed herein, the subject may have an implantable cardioverter defibrillator (ICD). In illustrative examples of this type, the administration of a CD14 antagonist antigen-binding molecule suitably reduces the number of electrical shocks delivered by the ICD to the subject.

In any of the aspects and embodiments disclosed herein, the antigen-binding molecule may be selected from:
(i) an antibody that comprises: a) an antibody VL domain, or antigen binding fragment thereof, comprising L-CDR1, L-CDR2 and L-CDR3, wherein: L-CDR1 comprises, consists or consists essentially of the sequence RASESVDSFGNSFMH [SEQ ID NO: 7] (3C10 L-CDR1); L-CDR2 comprises, consists or consists essentially of the sequence RAANLES [SEQ ID NO: 8] (3C10 L-CDR2); and L-CDR3 comprises, consists or consists essentially of the sequence QQSYEDPWT [SEQ ID NO: 9] (3C10 L-CDR3); and b) an antibody VH domain, or antigen binding fragment thereof, comprising H-CDR1, H-CDR2 and H-CDR3, wherein: H-CDR1 comprises, consists or consists essentially of the sequence SYAMS [SEQ ID NO: 10] (3C10 H-CDR1); H-CDR2 comprises, consists or consists essentially of the sequence SISSGGTTYYPDNVKG [SEQ ID NO: 11] (3C10 H-CDR2); and H-CDR3 comprises, consists or consists essentially of the sequence GYYDYHY [SEQ ID NO: 12] (3C10 H-CDR3);
(ii) an antibody that comprises: a) an antibody VL domain, or antigen binding fragment thereof, comprising L-CDR1, L-CDR2 and L-CDR3, wherein: L-CDR1 comprises, consists or consists essentially of the sequence RASESVDSYVNSFLH [SEQ ID NO: 13] (28C5 L-CDR1); L-CDR2 comprises, consists or consists essentially of the sequence RASNLQS [SEQ ID NO: 14] (28C5 L-CDR2); and L-CDR3 comprises, consists or consists essentially of the sequence QQSNEDPTT [SEQ ID NO: 15] (28C5 L-CDR3); and b) an antibody VH domain, or antigen binding fragment thereof, comprising H-CDR1, H-CDR2 and H-CDR3, wherein: H-CDR1 comprises, consists or consists essentially of the sequence SDSAWN [SEQ ID NO: 16] (28C5 H-CDR1); H-CDR2 comprises, consists or consists essentially of the sequence YISYSGSTSYNPSLKS [SEQ ID NO: 17] (28C5 H-CDR2); and H-CDR3 comprises, consists or consists essentially of the sequence GLRFAY [SEQ ID NO: 18] (28C5 H-CDR3);
(iii) an antibody that comprises: a) an antibody VL domain, or antigen binding fragment thereof, comprising L-CDR1, L-CDR2 and L-CDR3, wherein: L-CDR1 comprises, consists or consists essentially of the sequence RASESVDSYVNSFLH [SEQ ID NO: 13] (IC14 L-CDR1); L-CDR2 comprises, consists or consists essentially of the sequence RASNLQS [SEQ ID NO: 14] (IC14 L-CDR2); and L-CDR3 comprises, consists or consists essentially of the sequence QQSNEDPYT [SEQ ID NO: 27] (IC14 L-CDR3); and b) an antibody VH domain, or antigen binding fragment thereof, comprising H-CDR1, H-CDR2 and H-CDR3, wherein: H-CDR1 comprises, consists or consists essentially of the sequence SDSAWN [SEQ ID NO: 16] (IC14 H-CDR1); H-CDR2 comprises, consists or consists essentially of the sequence YISYSGSTSYNPSLKS [SEQ ID NO: 17] (IC14 H-CDR2); and H-CDR3 comprises, consists or consists essentially of the sequence GLRFAY [SEQ ID NO: 18] (IC14 H-CDR3);
(iv) an antibody that comprises: a) an antibody VL domain, or antigen binding fragment thereof, comprising L-CDR1, L-CDR2 and L-CDR3, wherein: L-CDR1 comprises, consists or consists essentially of the sequence RASQDIKNYLN [SEQ ID NO: 19] (18E12 L-CDR1); L-CDR2 comprises, consists or consists essentially of the sequence YTSRLHS [SEQ ID NO: 20] (18E12 L-CDR2); and L-CDR3 comprises, consists or consists essentially of the sequence QRGDTLPWT [SEQ ID NO: 21] (18E12 L-CDR3); and b) an antibody VH domain, or antigen binding fragment thereof, comprising H-CDR1, H-CDR2 and H-CDR3, wherein: H-CDR1 comprises, consists or consists essentially of the sequence NYDIS [SEQ ID NO: 22] (18E12 H-CDR1); H-CDR2 comprises, consists or consists essentially of the sequence VIWTSGGTNYNSAFMS [SEQ ID NO: 23] (18E12 H-CDR2); and H-CDR3 comprises, consists or consists essentially of the sequence GDGNFYLYNFDY [SEQ ID NO: 24] (18E12 H-CDR3);
(v) an antibody that comprises: a) an antibody VL domain, or antigen binding fragment thereof, comprising L-CDR1, L-CDR2 and L-CDR3, wherein: L-CDR1 comprises, consists or consists essentially of the sequence QNVGSNVDWY [SEQ ID NO: 34] (F1024-1-3 L-CDR1); L-CDR2 comprises, consists or consists essentially of the sequence KASNRY [SEQ ID NO: 35] (F1024-1-3 L-CDR2); and L-CDR3 comprises, consists or consists essentially of the sequence MQSNTNPPW [SEQ ID NO: 36] (F1024-1-3 L-CDR3); and b) an antibody VH domain, or antigen binding fragment thereof, comprising H-CDR1, H-CDR2 and H-CDR3, wherein: H-CDR1 comprises, consists or consists essentially of the sequence DYAMN [SEQ ID NO: 37] (F1024-1-3 H-CDR1); H-CDR2 comprises, consists or consists essentially of the sequence WINTQTGKPTYADDF [SEQ ID NO: 38] (F1024-1-3 H-CDR2); and H-CDR3 comprises, consists or consists essentially of the sequence TYFCTRSTFYYSSYIY [SEQ ID NO: 39] (F1024-1-3 H-CDR3);
(vi) an antibody that comprises: a) an antibody VL domain, or antigen binding fragment thereof, comprising L-CDR1, L-CDR2 and L-CDR3, wherein: L-CDR1 comprises, consists or consists essentially of the sequence KASQNVGSNVD [SEQ ID NO: 40] (F1024 L-CDR1); L-CDR2 comprises, consists or consists essentially of the sequence KASNRYT [SEQ ID NO: 41] (F1024 L-CDR2); and L-CDR3 comprises, consists or consists essentially of the sequence MQSNTNPPWT [SEQ ID NO: 42] (F1024 L-CDR3); and b) an antibody VH domain, or antigen binding fragment thereof, comprising H-CDR1, H-CDR2 and H-CDR3, wherein: H-CDR1 comprises, consists or consists essentially of the sequence DYAMN [SEQ ID NO: 37] (F1024 H-CDR1); H-CDR2 comprises, consists or consists essentially of the sequence WINTQTGKPTYADDFKQ [SEQ ID NO: 43] (F1024 H-CDR2); and H-CDR3 comprises, consists or consists essentially of the sequence STFYYSSYIYGWYFDF [SEQ ID NO: 44] (F1024 H-CDR3);
(vii) an antibody that comprises: a) an antibody VL domain, or antigen binding fragment thereof, comprising L-CDR1, L-CDR2 and L-CDR3, wherein: L-CDR1 comprises, consists or consists essentially of the sequence RASESVDSYGNSFMH [SEQ ID NO: 45] (r18D11 L-CDR1); L-CDR2 comprises, consists or consists essentially of the sequence LASNLES [SEQ ID NO: 46] (r18D11 L-CDR2); and L-CDR3 comprises, consists or consists essentially of the sequence QQNNGDPYT [SEQ ID NO: 47] (r18D11 L-CDR3); and b) an antibody VH domain, or antigen binding fragment thereof, comprising H-CDR1, H-CDR2 and H-CDR3, wherein: H-CDR1 comprises, consists or consists essentially of the sequence TYALN [SEQ ID NO: 48] (r18D11 H-CDR1); H-CDR2, consists or consists essentially of comprises the sequence RIRSKSNNYTTYYADSVKD [SEQ ID NO: 49] (r18D11 H-CDR2); and H-CDR3 comprises, consists or consists essentially of the sequence PQSGTSFAY [SEQ ID NO: 50] (r18D11 H-CDR3); and
(viii) an antibody that comprises: a) an antibody VL domain, or antigen binding fragment thereof, comprising L-CDR1, L-CDR2 and L-CDR3, wherein: L-CDR1 comprises, consists or consists essentially of the sequence KASQYVGTNVA [SEQ ID NO: 51] (rMil2 L-CDR1); L-CDR2 comprises, consists or consists essentially of the sequence SASYRCS [SEQ ID NO: 52] (rMil2 L-CDR2); and L-CDR3 comprises, consists or consists essentially of the sequence QQYNTYVT [SEQ ID NO: 53] (rMil2 L-CDR3); and b) an antibody VH domain, or antigen binding fragment thereof, comprising H-CDR1, H-CDR2 and H-CDR3, wherein: H-CDR1 comprises, consists or consists essentially of the sequence TYWMN [SEQ ID NO: 54] (rMil2 H-CDR1); H-CDR2 comprises, consists or consists essentially of the sequence RIDPYDSETHYNQNFKD [SEQ ID NO: 55] (rMil2 H-CDR2); and H-CDR3 comprises, consists or consists essentially of the sequence KEGROWGAYFDY [SEQ ID NO: 56] (rMil2 H-CDR3).

In specific embodiments, the antigen-binding molecule is selected from:
(i) an antibody comprising:
a VL domain that comprises, consists or consists essentially of the sequence:

(3C10 VL)
[SEQ ID NO: 1]
QSPASLAVSLGQRATISCRASESVDSFGNSFMHWYQQKAGQPPKSSIYRAANLESGIPARFSGSGSRT
DFTLTINPVEADDVATYFCQQSYEDPWTFGGGTKLGNQ;

and
a VH domain that comprises, consists or consists essentially of the sequence:

(3C10 VH)
[SEQ ID NO: 2]
LVKPGGSLKLSCVASGFTFSSYAMSWVRQTPEKRLEWVASISSGGTTYYPDNVKGRFTISRDNARNILY
LQMSSLRSEDTAMYYCARGYYDYHYWGQGTTLTVSS;

(ii) an antibody comprising:
a VL domain that comprises, consists or consists essentially of the sequence:

(28C5 VL)
[SEQ ID NO: 3]
QSPASLAVSLGQRATISCRASESVDSYVNSFLHWYQQKPGQPPKLLIYRASNLQS
GIPARFSGSGSRTDFTLTINPVEADDVATYCCQQSNEDPTTFGGGTKLEIK;

and
a VH domain that comprises, consists or consists essentially of the sequence:

(28C5 VH)
[SEQ ID NO: 4]
LQQSGPGLVKPSQSLSLTCTVTGYSITSDSAWNWIRQFPGNRLEWMGYISYSGSTSYNPSLKSRISITR
DTSKNQFFLQLNSVTTEDTATYYCVRGLRFAYWGQGTLVTVSA;

(iii) an antibody comprising:
a VL domain that comprises, consists or consists essentially of the sequence:

(18E12 VL)
[SEQ ID NO: 5]
QTPSSLSASLGDRVTISCRASQDIKNYLNWYQQPGGTVKVLIYYTSRLHSGVPSRFSGSGSGTDYSLTI
SNLEQEDFATYFCQRGDTLPWTFGGGTKLEIK;

and
a VH domain that comprises, consists or consists essentially of the sequence:

(18E12 VH)
[SEQ ID NO: 6]
LESGPGLVAPSQSLSITCTVSGFSLTNYDISWIRQPPGKGLEWLGVIWTSGGTNYNSAFMSRLSITKDN
SESQVFLKMNGLQTDDTGIYYCVRGDGNFYLYNFDYWGQGTTLTVSS;

(iv) an antibody comprising:
a VL domain that comprises, consists or consists essentially of the sequence:

(F1024-1-3 VL)
[SEQ ID NO: 57]
YIVMTQTPTSISISVGERVTMNCKASQNVGSNVDWYQQKTGQSPKLLIYKASNRYTGVPDRFTGSGSG
TDFTFTISNMQAVDLAVYYCMQSNTNPPWTFGGGTKLELKRA;

and
a VH domain that comprises, consists or consists essentially of the sequence:

(F1024-1-3 VH)
[SEQ ID NO: 58]
EVKLLESGGGLVQPSQTLSISCKASGYTFTDYAMNWVKQAPGDGLKWMGWINTQTGKPTYADDFKQ
RFVFSLETSASTAYLQINNLNIEDTATYFCTRSTFYYSSYIYGWYFDFWGPGTMVTVSS;

(v) an antibody comprising:
a VL domain that comprises, consists or consists essentially of the sequence:

(F1024 VL)
[SEQ ID NO: 59]
DIVMTQSPTSISISVGERVTMNCKASQNVGSNVDWYQQKTGQSPKLLIYKASNRYTGVPDRFTGSGS
GTDFTFTISNMQAVDLAVYYCMQSNTNPPWTFGGGTKLELKRA;

and
a VH domain that comprises, consists or consists essentially of the sequence:

(F1024 VH)
[SEQ ID NO: 60]
QIQLVQSGPELKKPGESVKISCKASGYTFTDYAMNWVKQAPGNGLKWMGW
INTQTGKPTYADDFKQRFVFSLETSASTAYLQINNLNIEDTATYFCTRST
FYYSSYIYGWYFDFWGPGTMVTVSS.

(vi) an antibody comprising:
a VL domain that comprises, consists or consists essentially of the sequence:

(r18D11 VL)
[SEQ ID NO: 61]
NIVLTQSPASLAVSLGQRATISCRASESVDSYGNSFMHWYQQKPGQPPKL
LIYLASNLESGVPARFSGSGSRTDFTLTIDPVEADDVATYYCQQNNGDPY
TFGGGTKLEIIR;

and
a VH domain that comprises, consists or consists essentially of the sequence:

(r18D11 VH)
[SEQ ID NO: 62]
EVQLVESGGGLMQPKGSLKLSCAASGFTFKTYALNWVRQAPGTGLEWVAR
IRSKSNNYTTYYADSVKDRFTISRDDSQNMLYLQMNNLKTEDTAMYYCVR
PQSGTSFAYWGQGTLVTVSA;

and
(vii) an antibody comprising:
a VL domain that comprises, consists or consists essentially of the sequence:

(rMil2 VL)
[SEQ ID NO: 63]
DIVMTQSQKFMSTSVGDRVSVTCKASQYVGTNVAWYQQKPGQSPKALIQS
ASYRCSGVPDRFTGSGSGTDFTLTISNVQSEDLADYFCQQYNTYVTFGGG
TKLELKR;

and
a VH domain that comprises, consists or consists essentially of the sequence:

(rMil2 VH)
[SEQ ID NO: 64]
QVRLQQPGAELVRPGASVKLSCKASGYTFTTYWMNWVKQRPEDGLEWIGR
IDPYDSETHYNQNFKDKAILTVDKSSSTAYMQLSSLTYEDSAVYYCTRKE
GRQWGAYFDYWGQGTTLTVSS.

The antigen-binding molecule may be humanized or chimeric.

Representative examples of the antigen-binding molecule comprise a light chain and a heavy chain, wherein:
the light chain comprises, consists or consists essentially of the amino acid sequence:

[SEQ ID NO: 25]
METDTILLWVLLLWVPGSTGDIVLTQSPASLAVSLGQRATISCRASESVD
SYVNSFLHWYQQKPGQPPKLLIYRASNLQSGIPARFSGSGSRTDFTLTIN
PVEADDVATYYCQQSNEDPYTFGGGTKLEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC;

and
the heavy chain comprises, consists or consists essentially of the amino acid sequence:

[SEQ ID NO: 26]
MKVLSLLYLLTAIPGILSDVQLQQSGPGLVKPSQSLSLTCTVTGYSITSD
SAWNWIRQFPGNRLEWMGYISYSGSTSYNPSLKSRISITRDTSKNQFFLQ
LNSVTTEDTATYYCVRGLRFAYWGQGTLVTVSSASTKGPSVFPLAPCSRS
TSESTAALGCLVKDYFPEPVTVSWNSGALTSGVHTFPAVLQSSGLYSLSS
VVTVPSSSLGTKTYTCNVDHKPSNTKVDKRVESKYGPPCPSCPAPEFLGG
PSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYT
QKSLSLSLGK.

In a specific embodiment, the antigen-binding molecule is the IC14 antibody.

In any of the aspects and embodiments disclosed herein, the subject is suitably a mammal, representative examples of which include humans, canines, felines, equines, bovines, ovines and porcines. In preferred embodiments, the subject is a human.

In any of the aspects and embodiments disclosed herein, the CD14 antagonist antigen-binding molecule may be administered systemically, or locally to the heart (e.g., left ventricle) of the subject.

In any of the aspects and embodiments disclosed herein, the methods suitably comprise administering an effective amount of the CD14 antagonist antigen-binding molecule.

In any of the aspects and embodiments disclosed herein, the CD14 antagonist antigen-binding molecule may be administered at a daily dose of between about 0.1 mg/kg and 50 mg/kg (and all one tenth integer mg/kg units in between), or between about 0.2 mg/kg and 40 mg/kg (and all one tenth integer mg/kg units in between), or between about 0.5 mg/kg and 40 mg/kg (and all one tenth integer mg/kg units in between), or between about 1 mg/kg and 30 mg/kg (and all integer mg/kg units in between), or between about 2 mg/kg and 20 mg/kg (and all integer mg/kg units in between), or between about 4 mg/kg and 15 mg/kg (and all integer mg/kg units in between), or between about 5 mg/kg and 10 mg/kg (and all integer mg/kg units in between). The daily dose is suitably administered in a single dose or in two doses.

In any of the aspects and embodiments disclosed herein, the CD14 antagonist antigen-binding molecule may be administered at a weekly dose of between about 1 mg/kg and 30 mg/kg (and all integer mg/kg units in between), or between about 2 mg/kg and 20 mg/kg (and all integer mg/kg units in between), or between about 4 mg/kg and 15 mg/kg (and all integer mg/kg units in between), or between about 5 mg/kg and 10 mg/kg (and all integer mg/kg units in between).

In any of the aspects and embodiments disclosed herein, the CD14 antagonist antigen-binding molecule is administered to the subject over a period of about 1 day, or over a period of about 2 days, or over a period of about 3 days, or over a period of about 4 days, or over a period of about 5 days, or over a period of about 6 days, over a period of about 1 week, or over a period of about 2 weeks, or over a period of about 3 weeks, or over a period of about 4 weeks, or over a period of about 5 weeks, or over a period of about 6 weeks, or over a period of about 2 months, or over a period of about 3 months, or over a period of about 4 months, or over a period of about 5 months, or over a period of about 6 months, or over a period of about 7 months, or over a period of about 8 months, or over a period of about 9 months, or over a period of about 10 months, or over a period of about 11 months, or over a period of about 1 year, or over a period of about 14 months, or over a period of about 16 months, or over a period of about 18 months, or over a period of about 20 months, or over a period of about 22 months, or over a period of about 2 years.

In specific embodiments, the subject is one that has not been diagnosed with autoimmune cardiomyopathy.

In specific embodiments, the subject is one that has not undergone cardiopulmonary bypass surgery.

In specific embodiments, the subject is one that is not an acute myocardial infarction patient.

In any of the aspects and embodiments disclosed herein, the methods may further comprise concurrently administering one or more ancillary cardiomyopathy therapeutic agents to the subject, illustrative examples of which include angiotensin converting enzyme inhibitors (e.g., Enalipril, Lisinopril), angiotensin receptor blockers (e.g., Losartan, Valsartan), beta blockers (e.g., Lopressor, Toprol-XL), antiarrhythmic drugs (e.g., amiodarone (Cordarone, Pacerone), flecainide (Tambocor), ibutilide (Corvert) lidocaine (Xylocaine), procainamide (Procan, Procanbid), propafenone (Rythmol), quinidine, tocainide (Tonocarid)) digoxin, diuretics (e.g., Lasix; or Parkinson's disease therapies including, for example, levodopa, dopamine agonists (e.g., bromocriptine, pergolide, pramipexole, ropinirole, Piribedil, cabergoline, apomorphine, lisuride), statins (e.g., atorvastatin, fluvastatin, lovastatin, pitavastatin, pravastatin, rosuvastatin, simvastatin) MAO-B inhibitors (e.g., selegiline or rasagiline), amantadine, anticholinergics, antipsychotics (e.g., clozapine), cholinesterase inhibitors, modafinil, thiadiazolidindiones and non-steroidal anti-inflammatory drugs. In some of the same and other aspects or embodiments, the methods may further comprise performing a surgical procedure on the subject, wherein the surgical procedure is suitable for treating the cardiomyopathy. The surgical procedure may comprise implantation of a pacemaker, implantation of an implantable cardioverter-defibrillator (ICD), cardiac catheterization, revascularization, coronary artery bypass and/or heart transplantation. The surgical procedure may be performed before, at the same time as, or after administration of the CD14 antagonist antigen-binding molecule, optionally together with an ancillary cardiomyopathy therapeutic agent.

Still another aspect of the present disclosure provides a CD14 antagonist antigen-binding molecule for use in treating or alleviating at least one symptom of a cardiomyopathy, in treating or inhibiting development of a cardiomyopathy, in alleviating or inhibiting development of adverse cardiac remodeling in a cardiomyopathy, in improving cardiac function in a cardiomyopathy, in improving ventricular function in a cardiomyopathy, in improving atrial function in a cardiomyopathy, in reducing the number of premature ventricular contractions (PVCs), in treating or alleviating at least one symptom of atrial fibrillation, or in reducing the number of premature atrial contractions (PACs). In some embodiments, the CD14 antagonist antigen-binding molecule is used in combination with one or more ancillary cardiomyopathy therapeutic agents.

Disclosed in yet another aspect is a use of a CD14 antagonist antigen-binding molecule in the manufacture of a medicament for treating or alleviating at least one symptom of a cardiomyopathy, for treating or inhibiting development of a cardiomyopathy, for alleviating or inhibiting development of adverse cardiac remodeling in a cardiomyopathy, for improving cardiac function in a cardiomyopathy, for improving ventricular function in a cardiomyopathy, for improving atrial function in a cardiomyopathy, for reducing the number of premature ventricular contractions (PVCs), for treating or alleviating at least one symptom of atrial fibrillation, or for reducing the number of premature atrial contractions (PACs). In some embodiments, the CD14 antagonist antigen-binding molecule is used in combination with one or more ancillary cardiomyopathy therapeutic agents.

### BRIEF DESCRIPTION OF THE DRAWINGS

FIG. 1 is a schematic representation depicting cardiomyopathic disease progression in the Dsg2^{mut/mut} mouse model.

FIG. 2 is a graphical representation showing that early intervention in the form of anti-CD14 treatment of 8-week old Dsg2^{mut/mut} mice over an 8-week period restores the % LVEF to a level seen in wild-type animals.

FIG. 3 is a graphical representation showing that early intervention in the form of anti-CD14 treatment of 8-week old Dsg2^{mut/mut} mice over an 8-week period prevents right ventricular dysfunction by normalizing % RVFAC.

FIG. 4 is a graphical representation showing that early intervention in the form of anti-CD14 treatment of 8-week old Dsg2^{mut/mut} mice over an 8-week period reduces ectopic heart beats by decreasing the frequency of PVCs, as measured by ECG.

FIG. 5 is a graphical representation showing that early intervention in the form of anti-CD14 treatment of 8-week old Dsg2^{mut/mut} mice over an 8-week period restores the LVM to a level seen in wild-type animals, thereby minimizing hypertrophic remodeling.

FIG. 6 is a graphical representation showing that early intervention in the form of anti-CD14 treatment of 8-week old Dsg2^{mut/mut} mice over an 8-week period reduces myocardial injury as measured by histologic analysis of fibrosis.

FIG. 7 is a graphical representation showing that late intervention in the form of anti-CD14 treatment of 16-week old Dsg2^{mut/mut} mice over an 8-week period significantly improves % LVEF as compared to untreated animals.

FIG. 8 is a graphical representation showing that late intervention in the form of anti-CD14 treatment of 16-week old Dsg2^{mut/mut} mice over an 8-week period significantly mitigates against further deterioration of the % LVEF as compared to untreated animals.

FIG. 9 is a graphical representation showing that late intervention in the form of anti-CD14 treatment of 16-week old Dsg2^{mut/mut} mice over an 8-week period reduces myocardial injury as measured by histologic analysis of fibrosis.

FIG. 10 is a graphical representation showing that the hearts of Dsg2^{mut/mut} mice contain pro-inflammatory macrophages that co-express CCR2 and CD14. (A) Uniform Manifold Approximation and Projection (UMAP) clustering of the integrated SCRNA-seq datasets representing the identified cellular clusters from the hearts of Dsg2^{mut/mut}, which are colored by cell type (i.e., fibroblasts, endothelial cells, B cells, monocytes/macrophages, neutrophils, NK cells and T cells). (B) Average expression of CCR2 according to cell type. (C) Average expression of CD14 according to cell type.

FIG. 11 is a photographic representation showing Imaging of 68Ga-DOTA-ECL1i uptake in the hearts of wild type mice (A and B) and Dsg2^{mut/mut} mice (C and D) treated with isotype control antibody (IgG2A LALAPG) or anti-CD14 (Big 53 LALAP mAb) at 5 mg/kg 1 per week for 4 weeks. Treatment of the Dsg2^{mut/mut} mice commenced at 8 weeks of age when overt signs of disease pathology are manifested.

FIG. 12 is a graphical representation showing the percentage injected dose (% ID) of ⁶⁸Ga-DOTA-ECL1i per gram of heart tissue in wild type (WT) mice and Dsg2^{mut/mut} mice treated for 6 weeks with either IgG2A LALAPG isotype control or Big53 LALAPG anti-CD14 mAb.

FIG. 13 is a photographic representation showing that human LV tissue obtained from a patient with cardiac sarcoidosis contains CCR2+ monocytes and macrophages. (A) Immunostaining of LV tissue for CCR2 (red) and for CD68 (green). (B) Representative positron emission tomography (PET) images showing uptake of ¹⁸F-fluorodeoxyglucose (¹⁸F-FDG) and 68Ga-DOTA-ECL1i (gallium-68 radiolabeled CCR2 PET tracer) by the myocardium of a patient with active cardiac sarcoidosis.

Some figures and text contain color representations or entities. Color illustrations are available from the Applicant upon request or from an appropriate Patent Office. A fee may be imposed if obtained from a Patent Office.

### DETAILED DESCRIPTION

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e. to at least one) of the grammatical object of the article. By way of example, “an element” means one element or more than one element.

The term “about” as used herein refers to the usual error range for the respective value readily known to the skilled person in this technical field. Reference to “about” in connection with a value or parameter herein includes (and describes) embodiments that are directed to that value or parameter per se. In specific embodiments, the term “about” refers to a value or parameter (e.g., quantity, level, concentration, number, frequency, percentage, dimension, size, amount, weight or length) that varies by as much 15, 14, 13, 12, 11, 10, 9, 8, 7, 6, 5, 4, 3, 2 or 1% to a reference value or parameter.

As used herein, “and/or” refers to and encompasses any and all possible combinations of one or more of the associated listed items, as well as the lack of combinations when interpreted in the alternative (or).

The terms “active agent” and “therapeutic agent” are used interchangeably herein and refer to agents that prevent, reduce or ameliorate at least one symptom of a disease or disorder.

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same formulation. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

The term “adverse cardiac remodeling” refers to a group of molecular, cellular and interstitial changes that manifest as changes in the size, shape and function of the heart. These changes have a negative impact on cardiac function and eventually lead to heart failure. Representative examples of adverse cardiac remodeling include hypertrophy, thinning of the myocardium, scar formation of the myocardium, atrophy of the myocardium, reduced heart function, reduced contractile function of the heart muscle, heart failure progression and combinations thereof.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present disclosure include polyclonal and monoclonal antibodies as well as their fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known. Antigen-binding molecules also encompass dimeric antibodies, as well as multivalent forms of antibodies. In some embodiments, the antigen-binding molecules are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., 1984, Proc. Natl. Acad. Sci. USA 81:6851-6855). Also contemplated, are humanized antibodies, which are generally produced by transferring complementarity determining regions (CDRs) from heavy and light variable chains of a non-human (e.g., rodent, preferably mouse) immunoglobulin into a human variable domain. Typical residues of human antibodies are then substituted in the framework regions of the non-human counterparts. The use of antibody components derived from humanized antibodies obviates potential problems associated with the immunogenicity of non-human constant regions. General techniques for cloning non-human, particularly murine, immunoglobulin variable domains are described, for example, by Orlandi et al. (1989, Proc. Natl. Acad. Sci. USA 86:3833). Techniques for producing humanized monoclonal antibodies are described, for example, by Jones et al. (1986, Nature 321:522), Carter et al. (1992, Proc. Natl. Acad. Sci. USA 89:4285), Sandhu (1992, Crit. Rev. Biotech. 12:437), Singer et al. (1993, J. Immun. 150:2844), Sudhir (ed., Antibody Engineering Protocols, Humana Press, Inc. 1995), Kelley (“Engineering Therapeutic Antibodies,” in Protein Engineering: Principles and Practice Cleland et al. (eds.), pages 399-434 (John Wiley & Sons, Inc. 1996), and by Queen et al., U.S. Pat. No. 5,693,762 (1997). Humanized antibodies include “primatized” antibodies in which the antigen-binding region of the antibody is derived from an antibody produced by immunizing macaque monkeys with the antigen of interest. Also contemplated as antigen-binding molecules are humanized antibodies.

The term “antagonist antigen-binding molecule” is used in the broadest sense, and includes an antigen-binding molecule that inhibits or decreases the biological activity of an antigen to which the antigen-binding molecule binds (e.g., CD14). For example, an antagonist antigen-binding molecule may partially or completely block interaction between a receptor (e.g., CD14) and a ligand (e.g., a DAMP or PAMP), or may practically decrease the interaction due to tertiary structure change or down regulation of the receptor. Thus, a CD14 antagonist antigen-binding molecule encompasses antigen-binding molecules that bind to CD14 and that block, inhibit, nullify, antagonize, suppress, decrease or reduce (including significantly), in any meaningful degree, a CD14 agonist activity, including activation of downstream pathways such as Toll-like receptor (TLR) signaling pathways (e.g., TLR4 signaling pathway) and the TIR-domain-containing adapter-inducing IFN-β (TRIF) pathway, or elicitation of a cellular response (e.g., production of pro-inflammatory mediators including pro-inflammatory cytokines) to CD14 binding by a CD14 ligand (e.g., a DAMP or PAMP). In some examples, the antibody is monospecific and binds only to CD14. In other examples, the antibody is multispecific (e.g., bispecific) and binds to CD14 and at least one other antigen.

The term “antibody” herein is used in its broadest sense and specifically covers naturally occurring antibodies, monoclonal antibodies, polyclonal antibodies, multi-specific antibodies (e.g., bispecific antibodies), antibody fragments, which exhibit the desired immuno-interactivity. A naturally occurring “antibody” includes within its scope an immunoglobulin comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised specific CH domains (e.g., CH1, CH2 and CH3). Each light chain is comprised of a light chain variable region (abbreviated herein as VL) and a light chain constant region. The light chain constant region is comprised of one domain, CL. The VH and VL regions can be further subdivided into regions of hypervariability, termed complementary determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The constant regions of the antibodies may mediate the binding of an immunoglobulin to host tissues or factors, including various cells of the immune system (e.g., effector cells) and the first component (C1q) of the classical complement system. The antibodies can be of any isotype (e.g., IgG, IgE, IgM, IgD, IgA and IgY), class (e.g., IgG1, IgG2, IgG3, IgG4, IgA1 and IgA2), subclass or modified version thereof (e.g., IgG1 isotype, which carries L234A and L235A double mutations (IgG1-LALA)). The antibodies can be of any species, chimeric, humanized or human. In other embodiments, the antibody is a homomeric heavy chain antibody (e.g., camelid antibodies) which lacks the first constant region domain (CH1) but retains an otherwise intact heavy chain and is able to bind antigens through an antigen-binding domain. The variable regions of the heavy and light chains in the antibody-modular recognition domain (MRD) fusions will contain a functional binding domain that interacts with an antigen of interest.

The “variable domain” (variable domain of a light chain (VL), variable domain of a heavy chain (VH)) as used herein denotes each of the pair of light and heavy chain domains which are involved directly in binding the antibody to the antigen. The variable light and heavy chain domains have the same general structure and each domain comprises four FRs whose sequences are widely conserved, connected by three CDRs or “hypervariable regions”. The FRs adopt a β-sheet conformation and the CDRs may form loops connecting the β-sheet structure. The CDRs in each chain are held in their three-dimensional structure by the FRs and form together with the CDRs from the other chain the antigen binding site.

The term “antigen-binding portion” when used herein refer to the amino acid residues of an antibody which are responsible for antigen-binding generally, which generally comprise amino acid residues from the CDRs. Thus, “CDR” or “complementarity determining region” (also referred to as “hypervariable region”) are used interchangeably herein to refer to the amino acid sequences of the light and heavy chains of an antibody which form the three-dimensional loop structure that contributes to the formation of an antigen binding site. There are three CDRs in each of the variable regions of the heavy chain and the light chain, which are designated “CDR1”, “CDR2”, and “CDR3”, for each of the variable regions. The term “CDR set” as used herein refers to a group of three CDRs that occur in a single variable region that binds the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, 1987. J. Mol. Biol. 196:901-917; Chothia et al., 1989. Nature 342:877-883) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan (1995. FASEB J. 9:133-139) and MacCallum (1996. J. Mol. Biol. 262 (5): 732-745). Still other CDR boundary definitions may not strictly follow one of these systems, but will nonetheless overlap with the Kabat CDRs, although they may be shortened or lengthened in light of prediction or experimental findings that particular residues or groups of residues or even entire CDRs do not significantly impact antigen binding.

As used herein, the term “framework region” or “FR” refers to the remaining sequences of a variable region minus the CDRs. Therefore, the light and heavy chain variable domains of an antibody comprise from N- to C-terminus the domains FR1, CDR1, FR2, CDR2, FR3, CDR3, and FR4. CDRs and FRs are typically determined according to the standard definition of Kabat, E. A., et al., Sequences of Proteins of Immunological Interest, 5th ed., Public Health Service, National Institutes of Health, Bethesda, Md. (1991) and/or those residues from a “hypervariable loop”.

As used herein, the terms “light chain variable region” (“VL”) and “heavy chain variable region” (VH) refer to the regions or domains at the N-terminal portion of the light and heavy chains respectively which have a varied primary amino acid sequence for each antibody. The variable region of an antibody typically consists of the amino terminal domain of the light and heavy chains as they fold together to form a three-dimensional binding site for an antigen. Several subtypes of VH and VL, based on structural similarities, have been defined, for example as set forth in the Kabat database.

The term “chimeric antibody” refers to antibodies that comprise heavy and light chain variable region sequences from one species and constant region sequences from another species, such as antibodies having murine heavy and light chain variable regions linked to human constant regions.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Thus, the FRs and CDRs of a humanized antibody need not correspond precisely to the parental (i.e., donor) sequences, e.g., a donor antibody CDR or the consensus framework may be mutagenized by substitution, insertion, and/or deletion of at least one amino acid residue so that the CDR or FR at that site does not correspond to either the donor antibody or the consensus framework. Typically, such mutations, however, will not be extensive and will generally avoid “key residues” involved in binding to an antigen. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, for example, Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will generally comprise at least a portion of an immunoglobulin constant region (Fc), typically that of a human immunoglobulin. For further details, see Jones et al. (1986. Nature 321:522-525), Riechmann et al. (1988. Nature 332:323-329) and Presta (1992. Curr. Op. Struct. Biol. 2:593-596). A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well known in the art. As used herein, the term “key residue” refers to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

As used herein, “Vernier” zone refers to a subset of framework residues that may adjust CDR structure and fine-tune the fit to antigen as described by Foote and Winter (1992. J. Mol. Biol. 224:487-499). Vernier zone residues form a layer underlying the CDRs and may impact on the structure of CDRs and the affinity of the antibody.

As used herein, the term “canonical” residue refers to a residue in a CDR or framework that defines a particular canonical CDR structure as defined by Chothia et al. (1987. J. Mol. Biol. 196:901-917; 1992. J. Mol. Biol. 227:799-817), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs to an “acceptor antibody”. In some embodiments, the donor antibody is an antibody from a species different from the antibody from which the FRs are obtained or derived. In the context of a humanized antibody, the term “donor antibody” refers to a non-human antibody providing one or more CDRs.

As used herein, the terms “acceptor” and “acceptor antibody” refer to an antibody providing at least 80%, at least 85%, at least 90%, at least 95%, at least 98%, or 100% of the amino acid sequences of one or more of the FRs. In some embodiments, the term “acceptor” refers to the antibody amino acid sequence providing the constant region(s). In other embodiments, the term “acceptor” refers to the antibody amino acid sequence providing one or more of the FRs and the constant region(s). In specific embodiments, the term “acceptor” refers to a human antibody amino acid sequence that provides at least 80%, preferably, at least 85%, at least 90%, at least 95%, at least 98%, or 100% of the amino acid sequences of one or more of the FRs. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, for example, derived or obtained from a germline antibody gene, a mature antibody gene, a functional antibody (e.g., antibodies well-known in the art, antibodies in development, or antibodies commercially available).

The term “human antibody”, as used herein, is intended to include antibodies having variable and constant regions derived from human germline immunoglobulin sequences. Human antibodies according to the present disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The terms “heavy chain variable region CDR1” and “H-CDR1” are used interchangeably, as are the terms “heavy chain variable region CDR2” and “H-CDR2”, the terms “heavy chain variable region CDR3” and “H-CDR3”, the terms “light chain variable region CDR1” and “L-CDR1”; the terms “light chain variable region CDR2” and “L-CDR2” and the terms “light chain variable region CDR3” and “L-CDR3” antibody fragment. Throughout the specification, complementarity determining regions (“CDR”) are defined according to the Kabat definition unless specified otherwise. The Kabat definition is a standard for numbering the residues in an antibody and it is typically used to identify CDR regions (Kabat et al., (1991), 5th edition, NIH publication No. 91-3242).

Antigen binding can be performed by “fragments” or “antigen-binding fragments” of an intact antibody. Herein, both terms are used interchangeably. Examples of binding fragments encompassed within the term “antibody fragment” of an antibody include a Fab fragment, a monovalent fragment consisting of the VL, VH, CL and CH1 domains; a F(ab′) 2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989. Nature 341:544-546), which consists of a VH domain; and an isolated complementary determining region (CDR). In a particular embodiment, the antibody of the present disclosure is an antigen-binding fragment that lacks all or a portion of the Fc region.

A “single chain variable Fragment (scFv)” is a single protein chain in which the VL and VH regions pair to form monovalent molecules (known as single chain Fv (scFv); see, e.g., Bird et al., 1988. Science 242:423-426; and Huston et al., 1988. Proc. Natl. Acad. Sci. 85:5879-5883). Although the two domains VL and VH are coded for by separate genes, they can be joined, using recombinant methods, by an artificial peptide linker that enables them to be made as a single protein chain. Such single chain antibodies include one or more antigen binding moieties. These antibody fragments are obtained using conventional techniques known to those of skill in the art, and the fragments are screened for utility in the same manner as are intact antibodies.

The term “monoclonal antibody” and abbreviations “MAb” and “mAb”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method. Monoclonal antibodies may be produced, for example, by a single clone of antibody-producing cells, including hybridomas. The term “hybridoma” generally refers to the product of a cell-fusion between a cultured neoplastic lymphocyte and a primed B- or T-lymphocyte which expresses the specific immune potential of the parent cell.

An antibody “that binds” an antigen of interest (e.g., CD14) is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody, oligopeptide or other organic molecule to its particular target protein as determined, for example, by fluorescence activated cell sorting (FACS) analysis, enzyme-linked immunosorbent assay (ELISA), immunoprecipitation or radioimmunoprecipitation (RIA). Thus, an antibody that antagonizes CD14 suitably inhibits or decreases production of pro-inflammatory mediators, including pro-inflammatory cytokines/chemokines. With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The specific region of the antigen to which the antibody binds is typically referred to as an “epitope”. The term “epitope” broadly includes the site on an antigen which is specifically recognized by an antibody or T-cell receptor or otherwise interacts with a molecule. Generally epitopes are of active surface groupings of molecules such as amino acids or carbohydrate or sugar side chains and generally may have specific three-dimensional structural characteristics, as well as specific charge characteristics. As will be appreciated by one of skill in the art, practically anything to which an antibody can specifically bind could be an epitope.

As used herein, “arrhythmia” refers to any of a group of conditions in which there is abnormal electrical activity in the heart. This can cause the heart beat to be too fast (tachycardia). Arrhythmia can affect the atria and/or the ventricles and occur at any age.

As used herein, the term “cardiomyopathy” refers to the deterioration of the function of the myocardium (i.e., the actual heart muscle) for any reason. Subjects with cardiomyopathy are often at risk of arrhythmia or sudden cardiac death or both. Cardiomyopathy can be acquired or inherited. Common symptoms include dyspnea and peripheral edema, and risks of having dangerous forms of irregular heart rate and sudden cardiac death are increased. Cardiomyopathy often leads to progressive heart failure, i.e., the incapacity of the cardiac pump to maintain sufficient blood flow to meet the basal bodily needs for oxygen. Based on the site of the lesion, cardiomyopathy is divided into primary cardiomyopathy in which the lesion is predominantly confined to heart; and secondary cardiomyopathy in which the myocardial lesions are involved in systemic disease. Furthermore, primary cardiomyopathy is classified into three types of hereditary, acquired and a mixed type of these. Examples of the hereditary type include hypertrophic cardiomyopathy, arrhythmogenic right ventricular cardiomyopathy, left ventricular noncompaction, glycogen storage disease (PPKA2, Danon's disease), conduction abnormalities, mitochondrial cardiomyopathy, ionic channel disorders (Long QT syndrome, Brugada syndrome, Short QT syndrome, catecholaminergic polymorphic ventricular tachycardia, and Asian SUNDS (nocturnal sudden death syndrome)). Examples of the acquired type include inflammatory cardiomyopathy (myocarditis), stress-induced cardiomyopathy (Tako-Tsubo cardiomyopathy), postpartum cardiomyopathy, tachycardia-induced cardiomyopathy, and cardiomyopathy of an infant born from an insulin-dependent mother. Examples of the mixed type include dilated cardiomyopathy and restrictive cardiomyopathy (left ventricular hypertrophy without enlargement).

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “effective amount”, in the context of treating a disease or condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the age, health and physical condition of the individual to be treated and whether symptoms of disease are apparent, the taxonomic group of individual to be treated, the formulation of the composition, the assessment of the medical situation, and other relevant factors. Optimal dosing schedules can be calculated from measurements of drug accumulation in the body of the subject. Optimum dosages may vary depending on the relative potency in an individual subject, and can generally be estimated based on EC50 values found to be effective in in vitro and in vivo animal models. Persons of ordinary skill can easily determine optimum dosages, dosing methodologies and repetition rates. It is expected that the amount will fall in a relatively broad range that can be determined through routine trials.

As used herein, the term “immune cell” refers to a cell involved in the innate or adaptive (acquired) immune systems. Exemplary innate immune cells include phagocytic cells such as neutrophils, monocytes and macrophages, Natural Killer (NK) cells, polymorphonuclear leukocytes such as neutrophils eosinophils and basophils and mononuclear cells such as monocytes, macrophages and mast cells. Immune cells with roles in acquired immunity include lymphocytes such as T-cells and B-cells.

As used herein, the term “inhibit”, “inhibits”, or “inhibiting” (and grammatical equivalents thereof), in the context of development of a state, disease, disorder or condition, refers to a delay in the appearance or onset of the state, disease, disorder or condition, reducing the manifestation of symptoms of the state, disease, disorder or condition, minimizing development of the state, disease, disorder or condition, and/or the lessening of symptoms upon onset of the state, disease, disorder or condition. The terms are not meant to imply complete abolition of disease and encompasses any type of prophylactic treatment that reduces the incidence of the condition or delays the onset and/or slows progression of the state, disease, disorder or condition.

By “isolated” is meant material that is substantially or essentially free from components that normally accompany it in its native state.

The term “ligand”, as used herein, refers to any molecule which is capable of binding a receptor.

As used herein the term “pro-inflammatory” refers to an inflammation-promoting effect.

As used herein the term “pro-inflammatory mediator” refers to molecules and cells having an inflammation-promoting effect and typically being functionally involved in the signaling pathways and the immune responses of the immune system. Such agents include, cytokines such as chemokines, interleukins (IL), lymphokines, and tumor necrosis factor (TNF) as well as growth factors. In specific embodiments, the pro-inflammatory mediator is a “pro-inflammatory cytokine”. Typically, pro-inflammatory cytokines include IL-1α, IL-1β, IL-6, and TNF-α, which are largely responsible for early responses. Other pro-inflammatory mediators include LIF, IFN-γ, IFN-β, IFN-α, OSM, CNTF, TGF-β, GM-CSF, TWEAK, IL-11, IL-12, IL-15, IL-17, IL-18, IL-19, IL-20, IL-8, IL-16, IL-22, IL-23, IL-31 and IL-32 (Tato et al., 2008. Cell 132:900; Cell 132:500, Cell 132:324). Pro-inflammatory mediators may act as endogenous pyrogens (IL-1, IL-6, TNF-α), up-regulate the synthesis of secondary mediators and pro-inflammatory cytokines by both macrophages and mesenchymal cells (including fibroblasts, epithelial and endothelial cells), stimulate the production of acute phase proteins, or attract inflammatory cells. In specific embodiments, the term “pro-inflammatory cytokine” relates to any one or more of TNF-α, IL-6, IFN-β, IL-1β and IL-8. In other specific embodiments, the term “pro-inflammatory cytokine” relates to any one or more of IL-1β, IL-6, TNF-α, IFN-γ and IFN-β.

The terms “subject”, “patient” and “individual” used interchangeably herein, refer to any subject, particularly a vertebrate subject, and even more particularly a mammalian subject, (e.g. a human, canine, feline, or equine subject) with a cardiomyopathy.

“Treating” or “treatment” of a state, disease, disorder or condition means an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease (e.g., maintaining a patient in remission), preventing spread of disease, delay or slowing of disease progression, amelioration or palliation of the disease state, diminishment of the reoccurrence of disease, and remission (whether partial or total), whether detectable or undetectable, or relieving the disease, i.e., causing regression of the state, disorder or condition or at least one of its clinical or subclinical symptoms. “Treating” and “Treatment” can also mean prolonging survival as compared to expected survival if not receiving treatment, includes: the benefit to an individual to be treated is either statistically significant or at least perceptible to the patient or to the physician. In one embodiment, treatment methods comprise administering to a subject an effective amount of a CD14 antagonist antigen-binding molecule and optionally consists of a single administration, or alternatively comprises a series of administrations.

Each embodiment described herein is to be applied mutatis mutandis to each and every embodiment unless specifically stated otherwise.

### 2. CD14 Antagonist Antigen-Binding Molecules for Use in Treating or Inhibiting Development of Cardiomyopathy

The present disclosure provides methods, uses and compositions that include a CD14 antagonist antigen-binding molecule for treating or alleviating at least one symptom of a cardiomyopathy, for treating or inhibiting development of a cardiomyopathy, for alleviating or inhibiting development of adverse cardiac remodeling in a cardiomyopathy, for improving cardiac function in a cardiomyopathy, for improving ventricular function in a cardiomyopathy, for improving atrial function in a cardiomyopathy, for reducing the number of premature ventricular contractions (PVCs), for treating or alleviating at least one symptom of atrial fibrillation, or for reducing the number of premature atrial contractions (PACs).

The present disclosure contemplates any CD14 antagonist antigen-binding molecule that binds to CD14, such as human CD14 (e.g. human mCD14 or sCD14) and blocks the binding of a CD14 ligand, suitably a DAMP or PAMP, to CD14 and/or that binds to CD14 and inhibits or decreases a CD14 agonist-mediated response resulting in the production of pro-inflammatory mediators, including the production of pro-inflammatory cytokines. Such CD14 antagonist antibodies are well known in the art and any can be utilized in the methods and uses of the present disclosure. In some embodiments, a CD14 antagonist antigen-binding molecule of the present disclosure inhibits binding of a CD14 agonist, suitably a DAMP or PAMP, to CD14 thus inhibiting or decreasing the production of pro-inflammatory cytokines. In illustrative examples of this type, the CD14 antagonist antigen-binding molecule is selected from the 3C10 antibody that binds to an epitope comprised in at least a portion of the region from amino acid 7 to amino acid 14 of human CD14 (van Voohris et al., 1983. J. Exp. Med. 158:126-145; Juan et al., 1995. J. Biol. Chem. 270 (29): 17237-17242), the MEM-18 antibody that binds an epitope comprised in at least a portion of the region from amino acid 57 to amino acid 64 of CD14 (Bazil et al., 1986. Eur. J. Immunol. 16 (12): 1583-1589; Juan et al., 1995. J. Biol. Chem. 270 (10): 5219-5224), the 4C1 antibody (Adachi et al., 1999. J. Endotoxin Res. 5:139-146; Tasaka et al., 2003. Am. J. Respir. Cell. Mol. Biol.; 2003. 29 (2): 252-258), the 28C5 and 23G4 antibodies that inhibit binding of LPS and suppress production of pro-inflammatory cytokines, the 18E12 antibody that partly inhibits binding of LPS and suppresses production of pro-inflammatory cytokines (U.S. Pat. Nos. 5,820,858, 6,444,206 and 7,326,569 to Leturcq et al.), the F1024-1-3 antibody (U.S. Pat. Appl. Pub. No. 2004/0091478 by Furusako et al.) and synthetic F1204 antibody (U.S. Pat. Appl. Pub. No. 2008/0286290 by Furusako et al.) which bind to an epitope comprised in at least a portion of the region from amino acid 269 to amino acid 315 of human CD14, and which inhibit binding of LPS and NF-κB activation as well as suppressing cytokine production, and the r18D11 and rMil2 antibodies (U.S. Pat. Appl. Pub. No. 2017/0107294 by Espevik et al.), that bind to the LPS-binding and/or signaling domains of human CD14 and inhibit LPS-induced release of pro-inflammatory cytokines. In some embodiments, a CD14 antagonist antigen-binding molecule of the present disclosure inhibits binding of CD14 to a TLR such as TLR4, thereby blocking CD14-agonist mediated response, illustrative examples of which include the F1024 antibody disclosed in International Publication WO2002/42333. Other CD14 antagonist antigen-binding molecules include the single-chain antibody scFv2F9 and the related human-mouse chimeric antibody Hm2F9 (Tang et al. 2007, Immunopharmacol Immunotoxicol. 29,375-386; and Shen et al., 2014, DNA Cell Biol. 33 (9): 599-604). Further examples of CD14 antagonist antigen-binding molecules include the anti-human CD14 18D11 IgG1 mAb, 18D11 IgG1 F (ab)′2 fragment and the chimeric r18D11 antibody (IgG2/4) (see e.g., Lau et al., 2013, J Immunol. 191:4769-4777). Each of the above references relating to CD14 antagonist antigen-binding molecules is incorporated herein by reference in its entirety. The CD14 antagonist antigen-binding molecule may be a full-length immunoglobulin antibody or an antigen-binding fragment of an intact antibody, representative examples of which include a Fab fragment, a F(ab′) 2 fragment, an Fd fragment consisting of the VH and CH1 domains, an Fv fragment consisting of the VL and VH domains of a single arm of an antibody, a single domain antibody (dAb) fragment (Ward et al., 1989. Nature 341:544-546), which consists of a VH domain; and an isolated CDR. Suitably, the CD14 antagonist antigen-binding molecule is a chimeric, humanized or human antibody.

In some embodiments, the CD14 antagonist antigen-binding molecule comprises a VH and VL of an antibody disclosed in U.S. Pat. No. 5,820,858:
(1) an antibody comprising:
a VL domain comprising, consisting or consisting essentially of the sequence:

(3C10 VL)
[SEQ ID NO: 1]
QSPASLAVSLGQRATISC RASESVDSFGNSFMH WYQQKAGQPPKSSIY
RAANLES GIPARFSGSGSRTDFTLTINPVEADDVATYFC QQSYEDPWT
FGGGTKLGNQ;

and
a VH domain comprising, consisting or consisting essentially of the sequence:

(3C10 VH)
[SEQ ID NO: 2]
LVKPGGSLKLSCVASGFTFS SYAMS WVRQTPEKRLEWVA
SISSGGTTYYPDNVKG RFTISRDNARNILYLQMSSLRSEDTAMYYCAR
GYYDYHY WGQGTTLTVSS;

(2) an antibody comprising:
a VL domain comprising, consisting or consisting essentially of the sequence:

(28C5 VL)
[SEQ ID NO: 3]
QSPASLAVSLGQRATISC RASESVDSYVNSFLH WYQQKPGQPPKLLIY
RASNLQS GIPARFSGSGSRTDFTLTINPVEADDVATYCC QQSNEDPTT
FGGGTKLEIK;

and
a VH domain comprising, consisting or consisting essentially of the sequence:

(28C5 VH)
[SEQ ID NO: 4]
LQQSGPGLVKPSQSLSLTCTVTGYSIT SDSAWN WIRQFPGNRLEWMG
YISYSGSTSYNPSLKS RISITRDTSKNQFFLQLNSVTTEDTATYYCVR
GLRFAY WGQGTLVTVSA;

and
(3) an antibody comprising:
a VL domain comprising, consisting or consisting essentially of the sequence:

(18E12 VL)
[SEQ ID NO: 5]
QTPSSLSASLGDRVTISC RASQDIKNYLN WYQQPGGTVKVLIY
YTSRLHS GVPSRFSGSGSGTDYSLTISNLEQEDFATYFC QRGDTLPWT
FGGGTKLEIK;

and
a VH domain comprising, consisting or consisting essentially of the sequence:

(18E12 VH)
[SEQ ID NO: 6]
LESGPGLVAPSQSLSITCTVSGFSLT NYDIS WIRQPPGKGLEWLG
VIWTSGGTNYNSAFMS RLSITKDNSESQVFLKMNGLQTDDTGIYYCVR
GDGNFYLYNFDY WGQGTTLTVSS;

In some embodiments, the CD14 antagonist antigen-binding molecule comprises a VH and VL of an antibody disclosed in U.S. Pat. Appl. Pub. No. 2004/0091478:
(1) an antibody comprising:
a VL domain comprising, consisting or consisting essentially of the sequence:

(F1024-1-3 VL)
[SEQ ID NO: 57]
YIVMTQTPTSISISVGERVTMNCKAS QNVGSNVDWY
QQKTGQSPKLLIY KASNRY
TGVPDRFTGSGSGTDFTFTISNMQAVDLAVYYC
MQSNTNPPW TFGGGTKLELKRA;

and
a VH domain comprising, consisting or consisting essentially of the sequence:

(F1024-1-3 VH)
[SEQ ID NO: 58]
EVKLLESGGGLVQPSQTLSISCKASGYTFT DYAMN
WVKQAPGDGLKWMG WINTQTGKPTYADD
FKQRFVFSLETSASTAYLQINNLNIEDTA TYFCTRSTFYYSSYIY
GWYFDFWGPGTMVTVSS.

In some embodiments, the CD14 antagonist antigen-binding molecule comprises a VH and VL of an antibody disclosed in U.S. Pat. Appl. Pub. No. 2008/0286290:
(1) an antibody comprising:
a VL domain comprising, consisting or consisting essentially of the sequence:

[SEQ ID NO: 59]
DIVMTQSPTSISISVGERVTMNC KASQNVGSNVD
WYQQKTGQSPKLLIY KASNRYT
GVPDRFTGSGSGTDFTFTISNMQAVDLAVYYC MQSNTNPPWT
FGGGTKLELKRA (F1024 VL);

and
a VH domain comprising, consisting or consisting essentially of the sequence:

[SEQ ID NO: 60]
QIQLVQSGPELKKPGESVKISCKASGYTFT DYAMN
WVKQAPGNGLKWMG WINTQTGKPTYADDFKQ
RFVFSLETSASTAYLQINNLNIEDTATYFCTR STFYYSSYIYGWYFDF
WGPGTMVTVSS (F1024 VH).

In some embodiments, the CD14 antagonist antigen-binding molecule comprises a VH and VL of an antibody disclosed in U.S. Pat. Appl. Pub. No. 2017/0107294:
(1) an antibody comprising:
a VL domain comprising, consisting or consisting essentially of the sequence:

[SEQ ID NO: 61)
NIVLTQSPASLAVSLGQRATISC RASESVDSYGNSFMH
WYQQKPGQPPKLLIY LASNLES
GVPARFSGSGSRTDFTLTIDPVEADDVATYYC QQNNGDPYT
FGGGTKLEIIR (r18D11 VL);

and
a VH domain comprising, consisting or consisting essentially of the sequence:

[SEQ ID NO: 62]
EVQLVESGGGLMQPKGSLKLSCAASGFTFK TYALN
WVRQAPGTGLEWVA RIRSKSNNYTTYYADSVKD
RFTISRDDSQNMLYLQMNNLKTEDTAMYYCVR PQSGTSFAY

and
(2) an antibody comprising:
a VL domain comprising, consisting or consisting essentially of the sequence:

[SEQ ID NO: 63]
DIVMTQSQKFMSTSVGDRVSVTC KASQYVGTNVA
WYQQKPGQSPKALIQ SASYRCS
GVPDRFTGSGSGTDFTLTISNVQSEDLADYFC QQYNTYVT
FGGGTKLELKR (rMil2 VL);

and
a VH domain comprising, consisting or consisting essentially of the sequence:

[SEQ ID NO: 64]
QVRLQQPGAELVRPGASVKLSCKASGYTFT TYWMN
WVKQRPEDGLEWIG RIDPYDSETHYNQNFKD
KAILTVDKSSSTAYMQLSSLTYEDSAVYYCTR KEGRQWGAYFDY
WGQGTTLTVSS (rMil2 VH).

Also contemplated are antigen-bind molecules that comprise the VL and VH CDR sequences of the above antibodies and related antibodies, representative embodiments of which include:
(1) an antibody that comprises: a) an antibody VL domain, or antigen binding fragment thereof, comprising L-CDR1, L-CDR2 and L-CDR3, wherein: L-CDR1 comprises, consists or consists essentially of the sequence RASESVDSFGNSFMH [SEQ ID NO: 7] (3C10 L-CDR1); L-CDR2 comprises, consists or consists essentially of the sequence RAANLES [SEQ ID NO: 8] (3C10 L-CDR2); and L-CDR3 comprises, consists or consists essentially of the sequence QQSYEDPWT [SEQ ID NO: 9] (3C10 L-CDR3); and b) an antibody VH domain, or antigen binding fragment thereof, comprising H-CDR1, H-CDR2 and H-CDR3, wherein: H-CDR1 comprises, consists or consists essentially of the sequence SYAMS [SEQ ID NO: 10] (3C10 H-CDR1); H-CDR2 comprises, consists or consists essentially of the sequence SISSGGTTYYPDNVKG [SEQ ID NO: 11] (3C10 H-CDR2); and H-CDR3 comprises, consists or consists essentially of the sequence GYYDYHY [SEQ ID NO: 12] (3C10 H-CDR3);
(2) an antibody that comprises: a) an antibody VL domain, or antigen binding fragment thereof, comprising L-CDR1, L-CDR2 and L-CDR3, wherein: L-CDR1 comprises, consists or consists essentially of the sequence RASESVDSYVNSFLH [SEQ ID NO: 13] (28C5 L-CDR1); L-CDR2 comprises, consists or consists essentially of the sequence RASNLQS [SEQ ID NO: 14] (28C5 L-CDR2); and L-CDR3 comprises, consists or consists essentially of the sequence QQSNEDPTT [SEQ ID NO: 15] (28C5 L-CDR3); and b) an antibody VH domain, or antigen binding fragment thereof, comprising H-CDR1, H-CDR2 and H-CDR3, wherein: H-CDR1 comprises, consists or consists essentially of the sequence SDSAWN [SEQ ID NO: 16] (28C5 H-CDR1); H-CDR2 comprises, consists or consists essentially of the sequence YISYSGSTSYNPSLKS [SEQ ID NO: 17] (28C5 H-CDR2); and H-CDR3 comprises, consists or consists essentially of the sequence GLRFAY [SEQ ID NO: 18] (28C5 H-CDR3);
(3) an antibody that comprises: a) an antibody VL domain, or antigen binding fragment thereof, comprising L-CDR1, L-CDR2 and L-CDR3, wherein: L-CDR1 comprises, consists or consists essentially of the sequence RASESVDSYVNSFLH [SEQ ID NO: 13] (IC14 L-CDR1); L-CDR2 comprises, consists or consists essentially of the sequence RASNLQS [SEQ ID NO: 14] (IC14 L-CDR2); and L-CDR3 comprises, consists or consists essentially of the sequence QQSNEDPYT [SEQ ID NO: 27] (IC14 L-CDR3); and b) an antibody VH domain, or antigen binding fragment thereof, comprising H-CDR1, H-CDR2 and H-CDR3, wherein: H-CDR1 comprises, consists or consists essentially of the sequence SDSAWN [SEQ ID NO: 16] (IC14 H-CDR1); H-CDR2 comprises, consists or consists essentially of the sequence YISYSGSTSYNPSLKS [SEQ ID NO: 17] (IC14 H-CDR2); and H-CDR3 comprises, consists or consists essentially of the sequence GLRFAY [SEQ ID NO: 18] (IC14 H-CDR3);
(4) an antibody that comprises: a) an antibody VL domain, or antigen binding fragment thereof, comprising L-CDR1, L-CDR2 and L-CDR3, wherein: L-CDR1 comprises, consists or consists essentially of the sequence RASQDIKNYLN [SEQ ID NO: 19] (18E12 L-CDR1); L-CDR2 comprises, consists or consists essentially of the sequence YTSRLHS [SEQ ID NO: 20] (18E12 L-CDR2); and L-CDR3 comprises, consists or consists essentially of the sequence QRGDTLPWT [SEQ ID NO: 21] (18E12 L-CDR3); and b) an antibody VH domain, or antigen binding fragment thereof, comprising H-CDR1, H-CDR2 and H-CDR3, wherein: H-CDR1 comprises, consists or consists essentially of the sequence NYDIS [SEQ ID NO: 22] (18E12 H-CDR1); H-CDR2 comprises, consists or consists essentially of the sequence VIWTSGGTNYNSAFMS [SEQ ID NO: 23] (18E12 H-CDR2); and H-CDR3 comprises, consists or consists essentially of the sequence GDGNFYLYNFDY [SEQ ID NO: 24] (18E12 H-CDR3);
(5) an antibody that comprises: a) an antibody VL domain, or antigen binding fragment thereof, comprising L-CDR1, L-CDR2 and L-CDR3, wherein: L-CDR1 comprises, consists or consists essentially of the sequence QNVGSNVDWY [SEQ ID NO: 34] (F1024-1-3 L-CDR1); L-CDR2 comprises, consists or consists essentially of the sequence KASNRY [SEQ ID NO: 35] (F1024-1-3 L-CDR2); and L-CDR3 comprises, consists or consists essentially of the sequence MQSNTNPPW [SEQ ID NO: 36] (F1024-1-3 L-CDR3); and b) an antibody VH domain, or antigen binding fragment thereof, comprising H-CDR1, H-CDR2 and H-CDR3, wherein: H-CDR1 comprises, consists or consists essentially of the sequence DYAMN [SEQ ID NO: 37] (F1024-1-3 H-CDR1); H-CDR2 comprises, consists or consists essentially of the sequence WINTQTGKPTYADDF [SEQ ID NO: 38] (F1024-1-3 H-CDR2); and H-CDR3 comprises, consists or consists essentially of the sequence TYFCTRSTFYYSSYIY [SEQ ID NO: 39] (F1024-1-3 H-CDR3);
(6) an antibody that comprises: a) an antibody VL domain, or antigen binding fragment thereof, comprising L-CDR1, L-CDR2 and L-CDR3, wherein: L-CDR1 comprises, consists or consists essentially of the sequence KASQNVGSNVD [SEQ ID NO: 40] (F1024 L-CDR1); L-CDR2 comprises, consists or consists essentially of the sequence KASNRYT [SEQ ID NO: 41] (F1024 L-CDR2); and L-CDR3 comprises, consists or consists essentially of the sequence MQSNTNPPWT [SEQ ID NO: 42] (F1024 L-CDR3); and b) an antibody VH domain, or antigen binding fragment thereof, comprising H-CDR1, H-CDR2 and H-CDR3, wherein: H-CDR1 comprises, consists or consists essentially of the sequence DYAMN [SEQ ID NO: 37] (F1024 H-CDR1); H-CDR2 comprises, consists or consists essentially of the sequence WINTQTGKPTYADDFKQ [SEQ ID NO: 43] (F1024 H-CDR2); and H-CDR3 comprises, consists or consists essentially of the sequence STFYYSSYIYGWYFDF [SEQ ID NO: 44] (F1024 H-CDR3);
(7) an antibody that comprises: a) an antibody VL domain, or antigen binding fragment thereof, comprising L-CDR1, L-CDR2 and L-CDR3, wherein: L-CDR1 comprises, consists or consists essentially of the sequence RASESVDSYGNSFMH [SEQ ID NO: 45] (r18D11 L-CDR1); L-CDR2 comprises, consists or consists essentially of the sequence LASNLES [SEQ ID NO: 46] (r18D11 L-CDR2); and L-CDR3 comprises, consists or consists essentially of the sequence QQNNGDPYT [SEQ ID NO: 47] (r18D11 L-CDR3); and b) an antibody VH domain, or antigen binding fragment thereof, comprising H-CDR1, H-CDR2 and H-CDR3, wherein: H-CDR1 comprises, consists or consists essentially of the sequence TYALN [SEQ ID NO: 48] (r18D11 H-CDR1); H-CDR2 comprises, consists or consists essentially of the sequence RIRSKSNNYTTYYADSVKD [SEQ ID NO: 49] (r18D11 H-CDR2); and H-CDR3 comprises, consists or consists essentially of the sequence PQSGTSFAY [SEQ ID NO: 50] (r18D11 H-CDR3); and
(8) an antibody that comprises: a) an antibody VL domain, or antigen binding fragment thereof, comprising L-CDR1, L-CDR2 and L-CDR3, wherein: L-CDR1 comprises, consists or consists essentially of the sequence KASQYVGTNVA [SEQ ID NO: 51] (rMil2 L-CDR1); L-CDR2 comprises, consists or consists essentially of the sequence SASYRCS [SEQ ID NO: 52] (rMil2 L-CDR2); and L-CDR3 comprises, consists or consists essentially of the sequence QQYNTYVT [SEQ ID NO: 53] (rMil2 L-CDR3); and b) an antibody VH domain, or antigen binding fragment thereof, comprising H-CDR1, H-CDR2 and H-CDR3, wherein: H-CDR1 comprises, consists or consists essentially of the sequence TYWMN [SEQ ID NO: 54] (rMil2 H-CDR1); H-CDR2 comprises, consists or consists essentially of the sequence RIDPYDSETHYNONFKD [SEQ ID NO: 55] (rMil2 H-CDR2); and H-CDR3 comprises, consists or consists essentially of the sequence KEGRQWGAYFDY [SEQ ID NO: 56] (rMil2 H-CDR3).

In some embodiments, the CD14 antagonist antigen-binding molecule is a humanized antibody. In illustrative examples of this type, the humanized CD14 antagonist antibody suitably comprises a donor CDR set corresponding to a CD14 antagonist antibody (e.g., one of the CD14 antagonist antibodies described above), and a human acceptor framework. The human acceptor framework may comprise at least one amino acid substitution relative to a human germline acceptor framework at a key residue selected from the group consisting of: a residue adjacent to a CDR; a glycosylation site residue; a rare residue; a canonical residue; a contact residue between heavy chain variable region and light chain variable region; a residue within a Vernier zone; and a residue in a region that overlaps between a Chothia-defined VH CDR1 and a Kabat-defined first heavy chain framework. Techniques for producing humanized mAbs are well known in the art (see, for example, Jones et al., 1986. Nature 321:522-525; Riechmann et al. 1988. Nature 332:323-329; Verhoeyen et al., 1988. Science 239:1534-1536; Carter et al., 1992. Proc. Natl. Acad. Sci. USA 89:4285-4289; Sandhu, J S., 1992. Crit. Rev. Biotech. 12:437-462, and Singer et al., 1993. J. Immunol. 150:2844-2857). A chimeric or murine monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. As simply transferring mouse CDRs into human FRs often results in a reduction or even loss of antibody affinity, additional modification might be required in order to restore the original affinity of the murine antibody. This can be accomplished by the replacement of one or more human residues in the FR regions with their murine counterparts to obtain an antibody that possesses good binding affinity to its epitope. See, for example, Tempest et al. (1991. Biotechnology 9:266-271) and Verhoeyen et al. (1988 supra). Generally, those human FR amino acid residues that differ from their murine counterparts and are located close to or touching one or more CDR amino acid residues would be candidates for substitution.

In some embodiments, the CD14 antagonist antibody is the IC14 antibody (Axtelle et al., 2001. J. Endotoxin Res. 7:310-314; and U.S. Pat. Appl. No. 2006/0121574, which are incorporated herein by reference in their entirety) or an antigen-binding fragment thereof. The IC14 antibody is a chimeric (murine/human) monoclonal antibody that specifically binds to human CD14. IC14 was derived from the murine 28C5 noted above and comprises an IgG4 heavy chain (see, U.S. Pat. Nos. 5,820,858, 6,444,206 and 7,326,569 to Leturcq et al., and Leturcq et al., 1996. J. Clin. Invest. 98:1533-1538). Thus, in one example, the CD14 antagonist antibody comprises the IC14 heavy chain and light chain CDRs, as described above. In another example, the CD14 antagonist antibody comprises the VL domain and a VH domain, wherein:
the VL domain comprises, consists or consists essentially of the amino acid sequence:

[SEQ ID NO: 25]
QSPASLAVSLGQRATISCRASESVDSYVNSFLHWYQQKPGQPPKLLIYR
ASNLQSGIPARFSGSGSRTDFTLTINPVEADDVATYYCQQSNEDPYTFG
GGTKLEIK;

and
the VH domain comprises, consists or consists essentially of the amino acid sequence:

[SEQ ID NO: 26]
LQQSGPGLVKPSQSLSLTCTVTGYSITSDSAWNWIRQFPGNRLEWMGYI
SYSGSTSYNPSLKSRISITRDTSKNQFFLQLNSVTTEDTATYYCVRGLR
FAYWGQGTLVTVSS;

or
the VL domain comprises, consists or consists essentially of the amino acid sequence:

[SEQ ID NO: 30]
DIVLTQSPASLAVSLGQRATISCRASESVDSYVNSFLHWYQQKPGQPPK
LLIYRASNLQSGIPARFSGSGSRTDFTLTINPVEADDVATYYCQQSNED
PYTFGGGTKLEIK;

and
the VH domain comprises, consists or consists essentially of the amino acid sequence:

[SEQ ID NO: 31]
DVQLQQSGPGLVKPSQSLSLTCTVTGYSITSDSAWNWIRQFPGNRLEWM
GYISYSGSTSYNPSLKSRISITRDTSKNQFFLQLNSVTTEDTATYYCVR
GLRFAYWGQGTLVTVSS.

In another example, the CD14 antagonist antibody comprises light chain and heavy chain of IC14, wherein:
the light chain comprises, consists or consists essentially of the amino acid sequence:

[SEQ ID NO: 28]
QSPASLAVSLGQRATISCRASESVDSYVNSFLHWYQQKPGQPPKLLIYR
ASNLQSGIPARFSGSGSRTDFTLTINPVEADDVATYYCQQSNEDPYTFG
GGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC;

and
the heavy chain comprises, consists or consists essentially of the amino acid sequence:

[SEQ ID NO: 29]
LQQSGPGLVKPSQSLSLTCTVTGYSITSDSAWNWIRQFPGNRLEWMGYI
SYSGSTSYNPSLKSRISITRDTSKNQFFLQLNSVTTEDTATYYCVRGLR
FAYWGQGTLVTVSSASTKGPSVFPLAPCSRSTSESTAALGCLVKDYFPE
PVTVSWNSGALTSGVHTFPAVLQSSGLYSLSSVVTVPSSSLGTKTYTCN
VDHKPSNTKVDKRVESKYGPPCPSCPAPEFLGGPSVFLFPPKPKDTLMI
SRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK;

or
the light chain comprises, consists or consists essentially of the amino acid sequence:

[SEQ ID NO: 32]
DIVLTQSPASLAVSLGQRATISCRASESVDSYVNSFLHWYQQKPGQPPK
LLIYRASNLQSGIPARFSGSGSRTDFTLTINPVEADDVATYYCQQSNED
PYTFGGGTKLEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC;

and
the heavy chain comprises, consists or consists essentially of the amino acid sequence:

[SEQ ID NO: 33]
DVQLQQSGPGLVKPSQSLSLTCTVTGYSITSDSAWNWIRQFPGNRLEWM
GYISYSGSTSYNPSLKSRISITRDTSKNQFFLQLNSVTTEDTATYYCVR
GLRFAYWGQGTLVTVSSASTKGPSVFPLAPCSRSTSESTAALGCLVKDY
FPEPVTVSWNSGALTSGVHTFPAVLQSSGLYSLSSVVTVPSSSLGTKTY
TCNVDHKPSNTKVDKRVESKYGPPCPSCPAPEFLGGPSVFLFPPKPKDT
LMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLG
K.

Additional CD14 antagonist antigen-binding molecules suitable for use in the methods disclosed herein can be identified by methods well known to those skilled in the art. These methods generally comprise determining whether an antibody is capable of directly antagonizing CD14. For example, the methods may involve determining whether an antibody is capable of inhibiting or decreasing the amount or agonist activity of CD14, wherein the ability to inhibit or decrease the amount or agonist activity of CD14 indicates that the antibody may be suitable for use in treating cardiomyopathy. In some embodiments, the antibody is contacted with CD14, or a cell that expresses CD14 on its surface, or a nucleic acid sequence from which CD14 is expressed, suitably in the presence of a CD14 agonist such as a DAMP or PAMP, wherein a decrease in the amount or agonist activity of CD14 in the presence of the agonist, when compared to a control, indicates that the antibody binds to CD14 and directly antagonizes CD14. A decrease or inhibition of CD14 agonist activity, includes for example inhibiting, or decreasing activation of, downstream pathways such as TLR signaling pathways (e.g., TLR4 signaling pathway) and the TRIF pathway, or elicitation of a cellular response (e.g., production of pro-inflammatory mediators including pro-inflammatory cytokines).

These methods may be carried out in vivo, ex vivo or in vitro. In particular, the step of contacting an antibody with CD14 or with a cell that expresses CD14 on its surface (e.g., immune cells) may be carried out in vivo, ex vivo or in vitro. The methods may be carried out in a cell-based or a cell-free system. For example, the method may comprise a step of contacting a cell expressing CD14 on its surface with an antibody and determining whether the contacting of the cell with the antibody leads to a decrease in the amount or agonist activity of CD14. In such a cell-based assay, the CD14 and/or the antibody may be endogenous to the host cell, may be introduced into a host cell or tissue, may be introduced into the host cell or tissue by causing or allowing the expression of an expression construct or vector or may be introduced into the host cell by stimulating or activating expression from an endogenous gene in the cell. In such a cell-based method, the amount of activity of CD14 may be assessed in the presence or absence of an antibody in order to determine whether the agent is altering the amount of CD14 in the cell, such as through regulation of CD14 expression in the cell or through destabilization of CD14 protein within the cell, or altering the CD14 agonist activity of the cell. The presence of a lower CD14 agonist activity or a decreased amount of CD14 on the cell surface in the presence of the antibody indicates that the antibody may be a suitable antagonist of CD14 for use in accordance with the present disclosure.

In some examples, it is further determined whether the antibody lacks substantial or detectable binding to another cellular component, suitably a binding partner of CD14, such as a CD14 binding partner that is either secreted (e.g., MD2) or located on the cell membrane (e.g., TLR4), to thereby determine that the antibody is a specific antagonist of CD14. In a non-limiting example of this type, the antibody is contacted in the presence of a CD14 agonist such as a DAMP or PAMP (1) with a wild-type cell that expresses CD14 on its surface (e.g., an immune cell such a macrophage), and (2) with a CD14 negative cell (e.g., an immune cell that is the same as in (1) but has a loss of function in the CD14 gene). If the antibody inhibits a CD14 agonist activity of the wild-type cell but not of the CD14 negative cell, this indicates that the antibody is a CD14 specific antagonist. Cells of this type may be constructed using routine procedures or animals.

In other examples, potential CD14 antagonist antigen-binding molecules are assessed in vivo, such as, for example, in an animal model. In such an in vivo model, the effects of the antibody may be assessed in the circulation (e.g., blood) or heart, or in other organs such as lung, liver, kidney, or the brain. In particular examples, models of cardiomyopathy are used to assess the activity of the antibody.

Exemplary CD14 antagonist antigen-binding molecules suitably effect a decrease in CD14 activity or levels of at least 5%, at least 10%, at least 25%, at least 50%, at least 60%, at least 75%, or at least 85% or more compared to in the absence of the antibody. in some examples, the antibody may result in a decrease in CD14 agonist activity or levels such that the agonist activity or level of CD14 is no longer detectable in the presence of the antibody. Such a decrease may be seen in the sample being tested or, for example where the method is carried out in an animal model.

Preferably, the antibody is a specific antagonist of CD14 as described above. However, this does not mean that a specific antagonist of CD14 has a complete absence of off-target antagonistic activity. In this regard, the specific antagonist of CD14 may have negligible or a minor direct binding and effect on other cellular components, such that the antagonism of the activity, signaling or expression of a non-CD14 cellular component, is less than less than 15%, less than 10%, less than 5%, less than 1%, or less than 0.1% of the direct binding and effect of that agent on the activity, signaling or expression of CD14.

Levels or amounts of CD14 may be measured by assessing expression of the CD14 gene. Gene expression may be assessed by looking at mRNA production or levels or at protein production or levels. Expression products such as mRNA and proteins may be identified or quantified by methods known in the art. Such methods may utilize hybridization to specifically identify the mRNA of interest. For example such methods may involve PCR or real-time PCR approaches. Methods to identify or quantify a protein of interest may involve the use of antibodies that bind that protein. For example, such methods may involve western blotting. Regulation of CD14 gene expression may be compared in the presence and absence of an antibody. Thus, antibodies can be identified that decrease CD14 gene expression compared to the level seen in the absence of the antibody. Such antibodies may be suitable antagonists of CD14 in accordance with the present disclosure.

The methods for identifying suitable antagonist antigen-binding molecules for use in accordance with the present disclosure may assess the agonist activity of CD14. For example, such a method may be carried out using peripheral blood mononuclear cells. Such cells will produce pro-inflammatory cytokines such as IL-1α, IL-6, TNF-α, IFN-β, IL-1β, IL-17 and IL-8 on response to stimulation with, for example, LPS. Methods may therefore comprise combining peripheral blood mononuclear cells with the antibody or a vehicle and adding LPS. The cells may then be incubated for an amount of time (e.g., 24 hours) to allow the production of pro-inflammatory mediators such as cytokines. The level of pro-inflammatory cytokines such as IL-1α, IL-6, TNF-α, IFN-β, IL-1β, IL-17 and IL-8 produced by the cells in that time period can then be assessed. If the antibody has anti-CD14 properties, then the production of such cytokines should be reduced compared to the vehicle-treated cells.

### 3. Ancillary Agents and Interventions

The CD14 antagonist antigen-binding molecule may be administered alone or in combination with one or more other active agents (also referred to as “ancillary therapeutic agents” and the like) or other interventions, such as agents and interventions that are useful for treating or inhibiting the development of a cardiomyopathy. The ancillary therapeutic agents may be any compound, molecule, or substance that exerts therapeutic effect to a subject in need thereof, suitably in the context of treating a cardiomyopathy.

Representative ancillary therapeutic agents suitable for the purposes of the present disclosure include, for example, fibrinolytic agents, beta blockers, high intensity statins (e.g., atorvastatin or rosuvastatin), angiotensin converting enzyme (ACE) inhibitors, angiotensin II receptor blockers, platelet inhibitors, thiadiazolidindiones, corticosteroids, anticonvulsants, immunosuppressants (e.g., cyclosporine, tacrolimus, prednisolone, hydrocortisone, sirolimus, everolimus, azathioprine, mycophenolic acid, methotrexate, basiliximab, daclizumab, rituximab, anti-thymocyte globulin, anti-lymphocyte globulin), antimicrobials, calcium channel blockers, digoxin, antiarrhythmics, anticoagulants, diuretics (e.g., spironolactone, eplerenone), exon-skipping therapies (e.g., eteplirsen, drisapersen), anti-myostatin antibodies (e.g., PF-06252616), anti-connective tissue growth factor antibodies (e.g., FG-3019), PDE5 inhibitors (e.g., tadalafil, sildenafil), PDE9 inhibitors, NF-κB inhibitors, stop codon read-through drugs (e.g., ataluren), utrophin modulators (e.g., SMT C1100, SMT022357), anti-fibrotic agents (e.g., halofuginone, angiotensin [1-7]), synthetic analogs of coenzyme Q₁₀ (e.g., idebenone), allogeneic cardiac cell therapy (e.g., CAP-1002; Toll-like receptor antagonists (e.g., IMO-8400); mineralocorticoid-receptor antagonists; β-adrenoceptor antagonists, resveratrol, and SIRT1 activators.

In some embodiments, the ancillary agent is a beta blocker (or beta-adrenoceptor antagonist). Suitable beta-blockers may be non-selective or beta-1 selective. Non-selective agents bind to both beta-1 and beta-2 receptors and induce antagonizing effects via both receptors. Non-limiting examples of non-selective beta blockers include propranolol, carvedilol, sotalol, and labetalol. Beta-1 receptor-selective blockers only bind to the beta-1 receptors and include, for example, atenolol, bisoprolol, metoprolol, and esmolol.

In other examples, the ancillary agent is a fibrinolytic agent, such as, for example, streptokinase, anistreplase or a tissue plasminogen activator (e.g., tenecteplase, reteplase or alteplase).

In further examples, the ancillary agent is a platelet inhibitor, such as aspirin, a P2Y12 inhibitors (e.g., ticlopidine, clopidogrel, ticagrelor or prasugrel) or a glycoprotein IIb/IIIa receptor antagonist.

In another example, the ancillary agent is an ACE inhibitor. Non-limiting examples of ACE inhibitors include benazepril, captopril, enalapril, fosinopril, Lisinopril, moexipril, perindopril, quinapril, ramipril and trandolapril.

In some embodiments, the ancillary agent is a thiadiazolidindione, representative examples of which include glycogen synthase kinase 3β (GSK3β) inhibitors, which are preferably selective, including reversible and irreversible inhibitors. Thiadiazolidindiones such as tideglusib or NPE100928, including derivatives thereof, can prevent electrical, molecular and structural changes associated with ACM, as disclosed for example in U.S. Pat. Appl. Pub. No. 2022/0249448, the contents of which are hereby incorporated by reference herein in their entirety.

In another example, the ancillary agent is a corticosteroid, illustrative examples of which include steroids, phenytoin, procainamide; quinine; glucocorticoids (e.g., deflazacort, vamorolone, VBP15, prednisone triamcinolone, methylprednisolone systemic, betamethasone, budesonide, prednisolone, hydrocortisone, dexamethasone, and/or cortisone.

In some embodiments, the ancillary agent is an immunosuppressants, representative examples of which include cyclosporine, tacrolimus, prednisolone, hydrocortisone, sirolimus, everolimus, azathioprine, mycophenolic acid, methotrexate, basiliximab, daclizumab, rituximab, anti-thymocyte globulin, and anti-lymphocyte globulin.

In another example, the ancillary agent is an anti-microbial, non-limiting examples of which include: penicillins (e.g., penicillin and amoxicillin); cephalosporins (e.g., cephalexin); macrolides (e.g., erythromycin, clarithromycin, and azithromycin); fluoroquinolones (e.g., levofloxacin and ofloxacin); sulfonamides (e.g., co-trimoxazole and trimethoprim); tetracyclines (e.g., tetracycline and doxycycline); and aminoglycosides (e.g., gentamicin and tobramycin)).

When two or more therapeutic agents are used in combination, the dosage of each therapeutic agent is commonly identical to the dosage of the agent when used independently. However, when a therapeutic agent interferes with the metabolism of others, the dosage of each therapeutic agent is properly adjusted. Alternatively, where the two or more therapeutic agents show synergistic effects, the dose of one or more may be reduced. Each therapeutic agent may be administered simultaneously or separately in an appropriate time interval.

When combination therapy is desired, the CD14 antagonist antibody is administered separately, simultaneously or sequentially with one or more ancillary agents or interventions. In some embodiments, this may be achieved by administering, such as systemically, a single composition or pharmacological formulation that includes both types of agent, or by administering two separate compositions or formulations at the same time, wherein one composition includes the CD14 antagonist antigen-binding molecule and the other the ancillary agent. In other embodiments, the treatment with the CD14 antagonist antigen-binding molecule may precede or follow the treatment with the ancillary agent by intervals ranging from minutes to hours or even days or weeks.

Where two or more agents are administered to a subject “in conjunction”, “in combination” or “concurrently” they may be administered in a single composition at the same time, or in separate compositions at the same time, or in separate compositions separated in time.

In another example, administration of the antigen-binding molecule is in conjunction with an intervention (e.g., surgical procedure), such as a percutaneous coronary intervention (PCI; also known as coronary angioplasty), coronary artery bypass including coronary artery bypass grafting (CABG), implantation of a pacemaker, implantation of an implantable cardioverter-defibrillator (ICD), cardiac catheterization, revascularization and heart transplantation.

In some situations, the antigen-binding molecule and ancillary agent(s) are administered within about 1-12 hours of each other or within about 2-6 hours of each other. In other situations, it may be desirable to extend the time period for treatment significantly, however, where one or more days (e.g., 1, 2, 3, 4, 5, 6, 7 or 8 days) lapse between the respective administrations. In embodiments where the ancillary agent is administered separately to the CD14 antagonist antigen-binding molecule, it will be understood that the ancillary agent can be administered by a method which is different to that of the administration method used for the CD14 antagonist antibody. In further embodiments, where an intervention (e.g., PCI) is performed on the subject, the antigen-binding molecule is administered to the subject within 72 hours of the PCI, such as at the time of or within 12, 24, 36 or 48 hours of the intervention.

### 4. Pharmaceutical Compositions

As described herein, a CD14 antagonist antigen-binding molecule, optionally in combination with at least one ancillary therapeutic agent, is useful for treating or alleviating at least one symptom of a cardiomyopathy, for treating or inhibiting development of a cardiomyopathy, for alleviating or inhibiting development of adverse cardiac remodeling in a cardiomyopathy, for improving cardiac function in a cardiomyopathy, for improving ventricular function in a cardiomyopathy, for improving atrial function in a cardiomyopathy, for reducing the number of premature ventricular contractions (PVCs), for treating or alleviating at least one symptom of atrial fibrillation, or for reducing the number of premature atrial contractions (PACs).

The CD14 antagonist antigen-binding molecule and optionally the ancillary agent(s) can be administered either by themselves suitably in the form of pharmaceutical compositions, which optionally comprise a pharmaceutically acceptable carrier. Thus, also provided herein are pharmaceutical compositions comprising a CD14 antagonist antigen-binding molecule, and optionally at least one ancillary therapeutic agent, for use in treating or inhibiting the development of a cardiomyopathy.

The CD14 antagonist antigen-binding molecules and optionally the ancillary therapeutic agent(s) may be formulated in a conventional manner using one or more pharmaceutically acceptable carriers, stabilizers or excipients (vehicles) to form a pharmaceutical composition as is known in the art, in particular with respect to protein active agents. Carrier(s) are “acceptable” in the sense of being compatible with the other ingredients of the composition and not deleterious to the recipient (e.g. patient) thereof. Suitable carriers typically include physiological saline or ethanol polyols such as glycerol or propylene glycol.

The antigen-binding molecule may be formulated as neutral or salt forms. Pharmaceutically acceptable salts include the acid addition salts (formed with free amino groups) and which are formed with inorganic acids such as hydrochloric or phosphoric acids, or such organic acids such as acetic, oxalic, tartaric and maleic. Salts formed with the free carboxyl groups may also be derived from inorganic bases such as sodium, potassium, ammonium, calcium, or ferric hydroxides, and organic bases as isopropylamine, trimethylamine, 2-ethylamino ethanol, histidine and procaine.

The compositions may be suitably formulated for systemic administration, including intravenous, intramuscular, subcutaneous, or intraperitoneal administration and conveniently comprise sterile aqueous solutions of the antigen-binding molecule, which are preferably isotonic with the blood of the recipient. Such formulations are typically prepared by dissolving solid active ingredient in water containing physiologically compatible substances such as sodium chloride, glycine, and the like, and having a buffered pH compatible with physiological conditions to produce an aqueous solution, and rendering said solution sterile. These may be prepared in unit or multi-dose containers, for example, sealed ampoules or vials.

The compositions may incorporate a stabilizer, such as for example polyethylene glycol, proteins, saccharides (e.g., trehalose), amino acids, inorganic acids and admixtures thereof. Stabilizers are used in aqueous solutions at the appropriate concentration and pH. The pH of the aqueous solution is adjusted to be within the range of 5.0-9.0, preferably within the range of 6-8. In formulating the antibody, anti-adsorption agent may be used. Other suitable excipients may typically include an antioxidant such as ascorbic acid. The compositions may be formulated as controlled release preparations which may be achieved through the use of polymer to complex or absorb the proteins. Appropriate polymers for controlled release formulations include for example polyester, polyamino acids, polyvinyl, pyrrolidone, ethylenevinylacetate, and methylcellulose. Another possible method for controlled release is to incorporate the antibody into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatin-microcapsules and poly(methylmethacylate) microcapsules, respectively, or in colloidal drug delivery systems, for example, liposomes, albumin microspheres, microemulsions, nanoparticles, and nanocapsules or in macroemulsions.

A CD14 antagonist antigen-binding molecule and optionally at least one ancillary agent may also be administered directly to the airways in the form of an aerosol. For use as aerosols, the antagonist antigen-binding molecules of the present disclosure in solution or suspension may be packaged in a pressurized aerosol container together with suitable propellants, for example, hydrocarbon propellants like propane, butane, or isobutane with conventional adjuvants. The materials disclosed herein may be administered in a non-pressurized form such as in a nebulizer or atomizer.

One of skill in the art will recognize that formulations are routinely designed according to their intended use, i.e. route of administration.

### 5. Methods of Treatment

The present disclosure also relates to the use of a pharmaceutical composition comprising a CD14 antagonist antigen-binding molecule, and optionally at least one ancillary therapeutic agent, in methods for treating or inhibiting the development of a cardiomyopathy. Cardiomyopathy is the measurable deterioration of the myocardium's ability to contract, leading to heart failure. The disease progresses over time with variable onset of arrhythmias and ventricle dysfunction. Electrocardiographic abnormalities can be found early in the disease and progress with age. Development of cardiomyopathy is characterized by initial diastolic dysfunction followed by eccentric hypertrophy.

In some aspects, the present disclosure provides methods of treating cardiomyopathy, comprising administering a pharmaceutical composition disclosed herein to a subject in need. In some aspects, the present disclosure provides a method of inhibiting development of cardiomyopathy, comprising administering a pharmaceutical composition disclosed herein to a subject in need. In some aspects, the present disclosure provides a method of delaying cardiomyopathy, comprising administering a pharmaceutical composition disclosed herein to a subject in need. In some aspects, the present disclosure provides a method of slowing progression of cardiomyopathy, comprising administering a pharmaceutical composition disclosed herein to a subject in need. In some aspects, the present disclosure provides a method of ameliorating cardiomyopathy, comprising administering a pharmaceutical composition disclosed herein to a subject in need.

Cardiomyopathies which may be treated or the development of which may be inhibited by a pharmaceutical composition disclosed herein include primary cardiomyopathies and secondary cardiomyopathies. Representative primary cardiomyopathies include: genetic cardiomyopathies, illustrative examples of which include hypertrophic cardiomyopathy, arrhythmogenic cardiomyopathy (ACM), left ventricular non-compaction, conduction system disease, ion channelopathy (e.g., long-QT syndrome), Brugada syndrome, catecholaminergic polymorphic ventricular tachycardia, short-QT syndrome and idiopathic ventricular fibrillation); mixed (genetic and non-genetic) cardiomyopathies, non-limiting examples of which include dilated cardiomyopathy and restrictive cardiomyopathy; and acquired cardiomyopathies such as myocarditis (e.g., viral myocarditis) and other inflammatory cardiomyopathies (e.g., Kawasaki disease and Chagas disease), stress (Tako-Tsubo) cardiomyopathy and peripartum cardiomyopathy. Representative secondary cardiomyopathies include: infiltrative cardiomyopathies associated with accumulation of abnormal substances between myocytes, non-limiting examples of which include cardiomyopathies resulting from amyloidosis, including primary, familial autosomal dominant, senile and secondary forms of amyloidosis, Gaucher disease, Hurler's disease and Hunter's disease; storage cardiomyopathies such a hemochromatosis, Fabry's disease, glycogen storage disease and Niemann-Pick disease; toxicity associated cardiomyopathies such as resulting from exposure to drugs, heavy metals and chemical agents, illustrative examples of which include such as chemotherapy cardiomyopathy (e.g., cardiomyopathies associated with exposure to: anthracyclines such as doxorubicin (Adriamycin) and daunorubicin; cyclophosphamide; and radiation) and alcoholic cardiomyopathy; endomyocardial cardiomyopathies including endomyocardial fibrosis, hypereosinophilic syndrome (Loeffler's endocarditis); granulomatous cardiomyopathies such as cardiac sarcoidosis; endocrine cardiomyopathies including those associated with diabetes mellitus, hyperthyroidism, hypothyroidism, hyperparathyroidism, pheochromocytoma and acromegaly; cardiofacial cardiomyopathies such as Noonan syndrome and lentiginosis; neuromuscular cardiomyopathies, which may be associated with accumulation of abnormal substances within myocytes, non-limiting examples of which include Friedreich's ataxia, Duchenne muscular dystrophy, Becker muscular dystrophy, Emery-Dreifuss muscular dystrophy, myotonic dystrophy, neurofibromatosis, and tuberous sclerosis; nutritional deficiency cardiomyopathies including cardiomyopathies associated with beriberi (thiamine), pallagra, scurvy, selenium, carnitine or kwashiorkor; autoimmune or collagen cardiomyopathies such as systemic lupus erythematosus, dermatomyositis, rheumatoid arthritis, scleroderma, and polyarteritis nodosa; and electrolyte imbalance cardiomyopathies. In specific embodiments, the cardiomyopathy is ACM, which is suitably arrhythmogenic right ventricular cardiomyopathy (ARVC). In other embodiments, the cardiomyopathy is a non-ACM cardiomyopathy.

In some embodiments, the subject has one of more mutations in a gene associated with ACM. Genes associated with ACM is for example a gene encoding a component of the desmosome. Genes encoding a component of the desmosome include for example, Plakophilin-2 (PKP2), Desmoplakin (DSP), Desmoglein-2 (DSG2), Desmocollin-2 (DSC2), or Plakoglobin (JUP). Other gene associated with ACM include, but are not limited to, Transmembrane protein 43 (TMEM43), Catenin Alpha 3 (CTNNA3), Desmin (DES), Lamin A/C (LMNA), Phospholamban (PLN), Ryanodine Receptor 2 (RYR2), Transforming growth factor beta-3 (TGFB3), Titin (TTN), Filamin C (FLNC), RNA Binding Motif Protein 20 (RBM20), Sodium Voltage-Gated Channel Alpha Subunit 5 (SCN5A), or BAG Cochaperone 3 (BAG3).

In some aspects, the cardiomyopathy is associated with an arrhythmia. In a representative embodiment of this type, the cardiomyopathy is ACM.

In some aspects, the subject has had an implantable cardioverter defibrillator (ICD) inserted due to a perceived risk of life-threatening arrhythmias or has previously suffered a cardiac arrest, sustained ventricular tachycardia, or episodes of worrisome syncope or pre-syncope.

In some aspects, the subject has begun to manifest clinical features of the disease, including frequent premature ventricular contractions (PVCs) or non-sustained ventricular tachycardia on ambulatory monitoring, electrocardiogram (ECG) abnormalities, such as an epsilon wave or precordial T-wave inversion, or evidence of cardiac dysfunction on imaging modalities, such as echocardiography and cardiac magnetic resonance imaging. Non-sustained ventricular tachycardia is defined as more than 3 beats of ventricular origin at a rate greater than 100 beats per minute that lasts less than 30 seconds in duration. When the rhythm lasts longer than 30 seconds or hemodynamic instability occurs in less than 30 seconds, it is considered sustained ventricular tachycardia. In some aspects, the patient has a damaging genetic variant that places them at risk of developing ACM and suffering from life-threatening arrhythmias.

Symptoms and/or complications of cardiomyopathy are well known in the art and include, but are not limited to, ventricular tachycardia, implantable cardioverter-defibrillator (ICD) shock, lung congestion, fluid retention, fatigue, heart murmurs, rapid heartbeat, arrhythmias, chest pain, light-headedness, fainting, dyspnea, peripheral edema, abdominal distention, embolization, myocardial fibrofatty infiltration, heart failure or sudden cardiac death (SCD).

The treatment or inhibition of development of cardiomyopathic symptoms may be measured by any means known in the art. For example, evaluation may include echocardiographic evaluation, cardia magnetic resonance imagining (MRI), cardiac MRI with late gadolinium enhancement. In particular, the LV size, thickness, volumes, ejection fraction (EF), and scar/inflammatory burden of the myocardium, as well as strain may be evaluated. Strain, change in LV dimension and volume, and scar burden are key measures.

In some aspects, the present disclosure provides a method of inhibiting development of adverse cardiac remodeling, comprising administering a pharmaceutical composition disclosed herein to a subject in need. In some aspects, the present disclosure provides a method of delaying adverse cardiac remodeling, comprising administering a pharmaceutical composition disclosed herein to a subject in need. In some aspects, the present disclosure provides a method of slowing the progression of adverse cardiac remodeling, comprising administering a pharmaceutical composition disclosed herein to a subject in need. In some aspects, the present disclosure provides a method of ameliorating adverse cardiac remodeling, comprising administering a pharmaceutical composition disclosed herein to a subject in need. In some embodiments, adverse cardiac remodeling is inhibited, delayed, or ameliorated for about 1 week, about 1 month, about 2 months, about 3 months, about 4 months, about 5 months, about 6 months, about 8 months, about 1 year, about 2 years, about 5 years, and/or about 10 years compared with the adverse cardiac remodeling in an untreated cardiomyopathic subject. In some embodiments, adverse cardiac remodeling is inhibited, delayed, or ameliorated by about 1%, about 5%, about 10%, about 20%, about 30%, about 40%, about 50%, about 60%, about 70%, about 80%, or about 90% compared with the adverse cardiac remodeling in an untreated cardiomyopathic subject. In some embodiments, this inhibition, delay, or amelioration of adverse cardiac remodeling is observed at the time points disclosed herein. In some embodiments, the adverse cardiac remodeling comprises an increase in fibrosis of the myocardium. In some embodiments, the adverse cardiac remodeling comprises a change in cardiac cavity diameter. In some embodiments, the adverse cardiac remodeling comprises a change in cardiac mass (hypertrophy and atrophy). In some embodiments, the adverse cardiac remodeling comprises a change in cardiac geometry (heart wall thickness and shape). In some embodiments, the adverse cardiac remodeling comprises a change in cardiac inflammatory infiltrate.

In some aspects, the present disclosure provides a method of inhibiting development of cardiac deterioration, comprising administering a pharmaceutical composition disclosed herein to a subject in need. In some aspects, the present disclosure provides a method of delaying cardiac deterioration, comprising administering a pharmaceutical composition disclosed herein to a subject in need. In some aspects, the present disclosure provides a method of slowing the progression of cardiac deterioration, comprising administering a pharmaceutical composition disclosed herein to a subject in need. In some aspects, the present disclosure provides a method of ameliorating cardiac deterioration, comprising administering a pharmaceutical composition disclosed herein to a subject in need. In some embodiments, cardiac deterioration is inhibited, delayed, or ameliorated for about 1 week, about 1 month, about 2 months, about 3 months, about 4 months, about 5 months, about 6 months, about 8 months, about 1 year, about 2 years, about 5 years, and/or about 10 years compared with the cardiac deterioration in an untreated cardiomyopathic subject. In some embodiments, cardiac deterioration is inhibited, delayed, or ameliorated by about 1%, about 5%, about 10%, about 20%, about 30%, about 40%, about 50%, about 60%, about 70%, about 80%, or about 90% compared with cardiac deterioration in an untreated cardiomyopathic subject. In some embodiments, this inhibition, delay, or amelioration of cardiac deterioration is observed at the time points disclosed herein. In some embodiments, the cardiac deterioration is a decrease in inotropy. In some embodiments, the cardiac deterioration is a decrease in lusitropy. In some embodiments, the cardiac deterioration is a thickening of the heart muscle. In some embodiments, the cardiac deterioration is cardiac hypertrophy.

In some embodiments, administration of the CD14 antagonist antigen-binding molecule improves cardiac function in a subject compared to an untreated cardiomyopathic subject. In some embodiments, cardiac function is improved for about 1 week, about 1 month, about 2 months, about 3 months, about 4 months, about 5 months, about 6 months, about 8 months, about 1 year, about 2 years, about 5 years, and/or about 10 years compared with the cardiac function of an untreated cardiomyopathic subject. In some embodiments, cardiac function is improved by about 1%, about 5%, about 10%, about 20%, about 30%, about 40%, about 50%, about 60%, about 70%, about 80%, or about 90% compared with the cardiac function of an untreated cardiomyopathie subject. In some embodiments, this improvement in cardiac function is observed at the time points disclosed herein. In some embodiments, the cardiac function improvement is an increase in inotropy. In some embodiments, the cardiac function improvement is an increase in lusitropy. In some embodiments, the cardiac function improvement is less thickening of the heart muscle. In some embodiments, the cardiac function improvement is less cardiac hypertrophy. In some embodiments, the cardiac function improvement is less premature ventricular contractions (PVCs). In some embodiments, the cardiac function improvement is less premature atrial contractions (PACs).

In some embodiments, administration of the CD14 antagonist antigen-binding molecule improves ventricular function in a subject compared to an untreated cardiomyopathie subject. In some embodiments, ventricular function is improved for about 1 week, about 1 month, about 2 months, about 3 months, about 4 months, about 5 months, about 6 months, about 8 months, about 1 year, about 2 years, about 5 years, and/or about 10 years compared with the ventricular function of an untreated cardiomyopathie subject. In some embodiments, ventricular function is improved by about 1%, about 5%, about 10%, about 20%, about 30%, about 40%, about 50%, about 60%, about 70%, about 80%, or about 90% compared with the ventricular function of an untreated cardiomyopathie subject. In some embodiments, this improvement in ventricular function is observed at the time points disclosed herein. In some embodiments, the ventricular function improvement is improved fractional shortening. In some embodiments, the ventricular function improvement is improved ejection fraction. In some embodiments, the ventricular function improvement is reduced end-diastolic volume. In some embodiments, the ventricular function improvement is decreased left ventricular mass. In some embodiments, the ventricular function improvement is a reduction in ventricular end diastolic diameter. In some embodiments, the ventricular function improvement is decreased arrhythmias. In some embodiments, the ventricular function improvement is reduced murmurs. In some embodiments, the ventricular function improvement is reduced heart rate. In some embodiments, the ventricular function improvement is normalizing of heart geometry. In some embodiments, the ventricular function improvement is less PVCs. PVCs may be measured by an electrocardiographic device such as for example a Holter monitor. A reduction of PVCs means a reduction of about ≥1%, about ≥2%, about ≥4%, about ≥5%, about ≥10%, about ≥15%, about ≥20%, about ≥25%, about ≥30%, about ≥35%, about ≥40%, about ≥45%, about ≥50%, or more compared to the number of PVC prior to treatment (i.e., the subjects baseline PVC rate). The number of PVCs and the reduction thereof can be measured in a period of about 24 hrs., about 48 hrs., about 72 hrs., about 96 hrs., about 120 hrs., or more. Alternatively, the number of PVCs and the reduction thereof can be measured in a period of about 1 day, about 2 days, about 3 days, about 4 days, about 5 days, about 6 days, about 7 days or more. The number of PVCs and the reduction thereof can be measured in a period of about 1 week, about 2 weeks, about 3 weeks, about 4 weeks, or more. The number of PVCs and the reduction thereof can be measured in a period of about 1 month, about 2 months, about 3 months, about 4 months, about 5 months, about 6 months, or more.

In some embodiments, administration of the CD14 antagonist antigen-binding molecule improves atrial function in a subject compared to an untreated cardiomyopathie subject. In some embodiments, atrial function is improved for about 1 week, about 1 month, about 2 months, about 3 months, about 4 months, about 5 months, about 6 months, about 8 months, about 1 year, about 2 years, about 5 years, and/or about 10 years compared with the atrial function of an untreated cardiomyopathie subject. In some embodiments, cardiac function is improved by about 1%, about 5%, about 10%, about 20%, about 30%, about 40%, about 50%, about 60%, about 70%, about 80%, or about 90% compared with the atrial function of an untreated cardiomyopathie subject. In some embodiments, this improvement in atrial function is observed at the time points disclosed herein. In some embodiments, the atrial function improvement is improved atrial reservoir function. In some embodiments, the atrial function improvement is improved atrial conduit function. In some embodiments, the atrial function improvement is improved atrial pump function. In some embodiments, the atrial function improvement is decreased left atrial mass. In some embodiments, the atrial function improvement is decreased arrhythmias. In some embodiments, the atrial function improvement is less PACs. A reduction of PACs means a reduction of about ≥1%, about ≥2%, about ≥4%, about ≥5%, about ≥10%, about ≥15%, about ≥20%, about ≥25%, about ≥30%, about ≥35%, about ≥40%, about ≥45%, about ≥50%, or more compared to the number of PAC prior to treatment (i.e., the subjects baseline PAC rate). The number of PACs and the reduction thereof can be measured in a period of about 24 hrs., about 48 hrs., about 72 hrs., about 96 hrs., about 120 hrs., or more. Alternatively, the number of PACs and the reduction thereof can be measured in a period of about 1 day, about 2 days, about 3 days, about 4 days, about 5 days, about 6 days, about 7 days or more. The number of PACs and the reduction thereof can be measured in a period of about 1 week, about 2 weeks, about 3 weeks, about 4 weeks, or more. The number of PACs and the reduction thereof can be measured in a period of about 1 month, about 2 months, about 3 months, about 4 months, about 5 months, about 6 months, or more.

In some aspects, the present disclosure provides a method of inhibiting development of atrial fibrillation, comprising administering a pharmaceutical composition disclosed herein to a subject in need. In some aspects, the present disclosure provides a method of delaying atrial fibrillation, comprising administering a pharmaceutical composition disclosed herein to a subject in need. In some aspects, the present disclosure provides a method of slowing the progression of atrial fibrillation, comprising administering a pharmaceutical composition disclosed herein to a subject in need. In some aspects, the present disclosure provides a method of ameliorating atrial fibrillation, comprising administering a pharmaceutical composition disclosed herein to a subject in need. In some embodiments, atrial fibrillation is inhibited, delayed, or ameliorated for about 1 week, about 1 month, about 2 months, about 3 months, about 4 months, about 5 months, about 6 months, about 8 months, about 1 year, about 2 years, about 5 years, and/or about 10 years compared with the atrial fibrillation in an untreated cardiomyopathie subject. In some embodiments, atrial fibrillation is inhibited, delayed, or ameliorated by about 1%, about 5%, about 10%, about 20%, about 30%, about 40%, about 50%, about 60%, about 70%, about 80%, or about 90% compared with the atrial fibrillation in an untreated cardiomyopathie subject. In some embodiments, this inhibition, delay, or amelioration of atrial fibrillation is observed at the time points disclosed herein.

In some aspects, the present disclosure provides a method of inhibiting development of fibrosis, comprising administering a pharmaceutical composition disclosed herein to a subject in need. In some aspects, the present disclosure provides a method of delaying the development of fibrosis, comprising administering a pharmaceutical composition disclosed herein to a subject in need. In some aspects, the present disclosure provides a method of slowing the progression of fibrosis, comprising administering a pharmaceutical composition disclosed herein to a subject in need. In some aspects, the present disclosure provides a method of ameliorating the development of fibrosis, comprising administering a pharmaceutical composition disclosed herein to a subject in need. In some embodiments, fibrosis is inhibited, delayed, or ameliorated for about 1 week, about 1 month, about 2 months, about 3 months, about 4 months, about 5 months, about 6 months, about 8 months, about 1 year, about 2 years, about 5 years, and/or about 10 years compared with the fibrosis in an untreated cardiomyopathie subject. In some embodiments, fibrosis is inhibited, delayed, or ameliorated by about 1%, about 5%, about 10%, about 20%, about 30%, about 40%, about 50%, about 60%, about 70%, about 80%, or about 90% compared with the fibrosis in an untreated cardiomyopathie subject. In some embodiments, this inhibition, delay, or amelioration of fibrosis is observed at the time points disclosed herein. In some embodiments, fibrosis is measured using histological techniques on a biopsy (e.g., hematoxylin and eosin (HE) and trichrome staining) where fibrosis can be assessed by determining the percentage of collagen present versus the total tissue area.

In some embodiments, the CD14 antagonist antigen-binding molecules disclosed herein are administered chronically. In some embodiments, the CD14 antagonist antigen-binding molecules disclosed herein are administered for about 6 months, for about 7 months, for about 8 months, for about 9 months, for about 10 months, for about 11 months, for about 1 year, for about 2 years, for about 3 years, for about 4 years, for about 5 years, for about 10 years or more. The CD14 antagonist antigen-binding molecules may be administered at any required dose and/or frequency disclosed herein.

In some embodiments, the CD14 antagonist antigen-binding molecules disclosed herein are administered until cardiomyopathie symptoms improve. In some embodiments, the CD14 antagonist antigen-binding molecules disclosed herein are administered until cardiomyopathie symptoms are ameliorated. In some embodiments, the CD14 antagonist antigen-binding molecules disclosed herein are administered until cardiomyopathie symptoms are delayed. In some embodiments, the CD14 antagonist antigen-binding molecules disclosed herein are administered until cardiomyopathie symptoms are cured.

In some embodiments, the CD14 antagonist antigen-binding molecules disclosed herein are administered before the patient begins to exhibit one or more cardiomyopathie symptoms. In some embodiments, the CD14 antagonist antigen-binding molecules disclosed herein are administered at the onset of cardiomyopathie symptoms. In some embodiments, the CD14 antagonist antigen-binding molecules disclosed herein are administered before the patient begins to exhibit cardiomyopathy. In some embodiments, the CD14 antagonist antigen-binding molecules disclosed herein are administered after manifestation of cardiomyopathy symptoms.

In some embodiments, the subject is determined to have abnormal echocardiograms compared to a non-cardiomyopathie subject. In some embodiments, the subject is determined to have changes in cardiac performance compared to a non-cardiomyopathie subject. In some embodiments, the subject is determined to have an increased P-R interval compared to non-cardiomyopathie subjects. In some embodiments, the subject is determined to have increased U waves compared to non-cardiomyopathie subjects. In some embodiments, the subject is determined to have wide QRS compared to non-cardiomyopathie subjects. In some embodiments, the subject is determined to have nonspecific ST-T changes compared to non-cardiomyopathie subjects. In some embodiments, the subject is determined to have sinus arrhythmias compared to non-cardiomyopathie subjects. In some embodiments, the subject is determined to have deep Q waves and elevated R waves precordially compared to non-cardiomyopathie subjects. In some embodiments, the subject is determined to have an abnormal tonic contraction (TC) compared to non-cardiomyopathie subjects. In some embodiments, tonic contraction results from a period of calcium dysregulation which manifests as sustained ion-driven myocyte contraction, resulting in echocardiographic appearance of left ventricle “underfilling” (Su et al. (2015, Pediatr. Cardiol. December 29). In some embodiments, the subject is determined to have an arrhythmia compared to non-cardiomyopathie subjects. In some embodiments, the subject is determined to have an sinus tachycardia compared to non-cardiomyopathie subjects. In some embodiments, the subject is determined to have systolic dysfunction compared to non-cardiomyopathie subjects. In some embodiments, the subject is determined to have diastolic dysfunction compared to non-cardiomyopathie subjects. In some embodiments, the subject is determined to have decreased mitral systolic wave velocity compared to non-cardiomyopathie subjects. In some embodiments, the subject is determined to have wall motion abnormalities compared to non-cardiomyopathie subjects. The heart function and/or characteristics may be measured using electrocardiogram.

In some embodiments, the subject is determined to have increased pro-inflammatory mediator, suitably pro-inflammatory cytokine, expression compared to a non-cardiomyopathie subject. In some embodiments, the subject is determined to have increased pro-inflammatory mediator, suitably pro-inflammatory cytokine, activity compared to a non-cardiomyopathie subject. In some embodiments, the pro-inflammatory cytokine is any pro-inflammatory cytokine, including but not limited to, OPN, LTβ4, TNF-α, interleukin-6 (IL-6) and soluble tumor necrosis factor-alpha receptor (sTNFαR). The expression or activity of pro-inflammatory cytokines in a subject may be measured through cytokine assays.

In some embodiments, the subject is determined to have decreased neuronal nitric oxide synthase (nNOS) protein expression compared to a non-cardiomyopathie subject. In some embodiments, the subject is determined to have decreased nNOS activity compared to a non-cardiomyopathie subject. In some embodiments, the subject is determined to have altered nNOS protein accumulation compared to a non-cardiomyopathie subject. In some embodiments, the subject is determined to have increased nNOS protein accumulation in the cytosol of skeletal muscle compared to a non-cardiomyopathie subject. In some embodiments, the subject is determined to have decreased nNOS protein accumulation in the sarcolemma of skeletal muscle compared to a non-cardiomyopathie subject. Protein accumulation may be assayed through histological techniques. Protein expression may be assayed through immunoprecipitation techniques. Protein activity may be measured by NOS catalytic assays.

In some embodiments, pharmaceutical compositions comprising a CD14 antagonist antigen-binding molecule and optionally at least one ancillary therapeutic agent (“active agents”) disclosed herein are for “systemic delivery”, meaning that the active agent(s) is (are) not delivered locally to a pathological site or a site of action. Instead, the active agent(s) is (are) absorbed into the bloodstream from the injection site, where the active agent(s) acts systemically or is transported to a site of action via the circulation. The active agent(s) may be administered by any suitable route, such as for example, orally, intravenously, intramuscularly, nasally, subcutaneously, and intra-rectally. In some embodiments, the active agent(s) is (are) for parenteral administration, preferably intravenous administration. In other embodiments, the active agent(s) is (are) for local administration to a pathological site to be treated (e.g., myocardial tissue). In these embodiments, the active agent(s) may be administered by injection, insertion or implantation directly into or onto or adjacent to the pathological site.

In some embodiments, the active agent(s) is (are) administered about monthly, and may be administered locally or intravenously. In some embodiments, the active agent(s) is (are) administered about weekly, and may be administered locally or intravenously. In some embodiments, the site of administration is not a pathological site, for example, is not the intended site of action.

In various embodiments, the plasma concentration of the active agent(s) does not change by more than a factor of about 100, or a factor of about 50, or a factor of about 10, or a factor of about 5, or a factor of about 3 over the course of a plurality of administrations, such as at least 2, at least about 5, or at least about 10 administrations of the formulation. The administrations are substantially evenly spaced, such as, for example, about daily, or about once per week, or from one to about five times per month, or about once every two months, or about once every three months. In some embodiments, the active agent(s) is (are) administered to the subject over a period of about 1 day, or over a period of about 2 days, or over a period of about 3 days, or over a period of about 4 days, or over a period of about 5 days, or over a period of about 6 days, over a period of about 1 week, or over a period of about 2 weeks, or over a period of about 3 weeks, or over a period of about 4 weeks, or over a period of about 5 weeks, or over a period of about 6 weeks, or over a period of about 2 months, or over a period of about 3 months, or over a period of about 4 months, or over a period of about 5 months, or over a period of about 6 months, or over a period of about 7 months, or over a period of about 8 months, or over a period of about 9 months, or over a period of about 10 months, or over a period of about 11 months, or over a period of about 1 year, or over a period of about 14 months, or over a period of about 16 months, or over a period of about 18 months, or over a period of about 20 months, or over a period of about 22 months, or over a period of about 2 years.

While one of skill in the art can determine the desirable dose in each case, a suitable effective dosage of a CD14 antagonist antigen-binding molecule for achieving a therapeutic benefit, may, for example, be in a range of about 0.1 mg and 50 mg (and all one tenth integer mg units in between), or between about 0.2 mg and 40 mg (and all one tenth integer mg units in between), or between about 0.5 mg and 40 mg (and all one tenth integer mg units in between), or between about 1 mg and 30 mg (and all integer mg units in between), or between about 2 mg and 20 mg (and all integer mg units in between), or between about 4 mg and 15 mg (and all integer mg units in between), or between about 5 mg and 10 mg (and all integer mg units in between) per kilogram body weight per day. In some embodiments, the CD14 antagonist antigen-binding molecule is administered at a low dose. In some embodiments, the CD14 antagonist antigen-binding molecule is administered at a dose between 1 mg per kilogram per body weight per day to about 9 mg per kilogram per body weight per day. In some embodiments, a suitable effective dosage of the CD14 antagonist antigen-binding molecule for achieving a therapeutic benefit, may, for example, be in a range of 0.1 mg and 50 mg (and all one tenth integer mg units in between), or between about 0.2 mg and 40 mg (and all one tenth integer mg units in between), or between about 0.5 mg and 40 mg (and all one tenth integer mg units in between), or between about 1 mg and 30 mg (and all integer mg units in between), or between about 2 mg and 20 mg (and all integer mg units in between), or between about 4 mg and 15 mg (and all integer mg units in between), or between about 5 mg and 10 mg (and all integer mg units in between) per kilogram body weight per week. Alternatively, if the condition of the recipient so requires, the doses may be administered as a continuous infusion.

In certain embodiments, the subject is a human, but in other embodiments may be a non-human mammal, such as a domesticated pet (e.g., dog or cat), or livestock or farm animal (e.g., horse, cow, sheep, or pig).

### 6. Representative Embodiments

1. A method of treating or alleviating at least one symptom of a cardiomyopathy in a subject, the method comprising, consisting or consisting essentially of administering to the subject a CD14 antagonist antigen-binding molecule.

2. The method of embodiment 1, wherein the at least one symptom is selected from ventricular tachycardia, implantable cardioverter-defibrillator (ICD) shock, lung congestion, fluid retention, fatigue, heart murmurs, rapid heartbeat, arrhythmias, chest pain, light-headedness, fainting, dyspnea, peripheral edema, abdominal distention, myocardial fibro-fatty infiltration, embolization, syncope, angina, exercise intolerance, orthopnea, heart failure and sudden cardiac death (SCD).

3. The method of embodiment 1 or embodiment 2, wherein the administration of the CD14 antagonist antigen-binding molecule improves one or more clinical parameters of the subject.

4. The method of embodiment 3, wherein the one or more clinical parameters are selected from reduced left ventricular ejection fraction, left ventricular hypertrophy, increased myocardial fibrosis, adverse cardiac remodeling, right ventricular dysfunction, aberrant heart geometry (e.g., increased right ventricular fractional area change), increased end-diastolic volume, increased frequency of premature ventricular contractions, and increased left ventricular mass.

5. A method of alleviating or inhibiting development of adverse cardiac remodeling in a subject with a cardiomyopathy, the method comprising, consisting or consisting essentially of administering to the subject a CD14 antagonist antigen-binding molecule.

6. A method of improving cardiac function in a subject with a cardiomyopathy, the method comprising, consisting or consisting essentially of administering to the subject a CD14 antagonist antigen-binding molecule.

7. The method of embodiment 6, wherein improving cardiac function comprises improving left ventricular function, improving fractional shortening, improving ejection fraction, reducing end-diastolic volume, decreasing left ventricular mass, decreasing arrhythmias, reducing frequency of heart murmurs, reducing heart rate, normalizing of heart geometry, or a combination thereof.

8. A method of improving ventricular function in a subject with a cardiomyopathy, the method comprising, consisting or consisting essentially of administering to the subject a CD14 antagonist antigen-binding molecule.

9. A method of improving atrial function in a subject with a cardiomyopathy, the method comprising, consisting or consisting essentially of administering to the subject a CD14 antagonist antigen-binding molecule.

10. The method of any one of embodiments 1 to 9, wherein the cardiomyopathy is a primary cardiomyopathy or a secondary cardiomyopathy.

11. The method of embodiment 10, wherein the primary cardiomyopathy is selected from genetic cardiomyopathies (e.g., hypertrophic cardiomyopathy, arrhythmogenic cardiomyopathy (ACM), left ventricular non-compaction, conduction system disease, ion channelopathy (e.g., long-QT syndrome), Brugada syndrome, catecholaminergic polymorphic ventricular tachycardia, short-QT syndrome and idiopathic ventricular fibrillation), mixed (genetic and non-genetic) cardiomyopathies (e.g., dilated cardiomyopathy and restrictive cardiomyopathy), and acquired cardiomyopathies (e.g., myocarditis such as viral myocarditis and other inflammatory cardiomyopathies such as Kawasaki disease and Chagas disease, stress (Tako-Tsubo) cardiomyopathy and peripartum cardiomyopathy).

12. The method of embodiment 10, wherein the secondary cardiomyopathy is selected from genetic cardiomyopathies, illustrative examples of which include hypertrophic cardiomyopathy, arrhythmogenic cardiomyopathy (ACM), left ventricular non-compaction, conduction system disease, ion channelopathy (e.g., long-QT syndrome), Brugada syndrome, catecholaminergic polymorphic ventricular tachycardia, short-QT syndrome and idiopathic ventricular fibrillation); mixed (genetic and non-genetic) cardiomyopathies, non-limiting examples of which include dilated cardiomyopathy and restrictive cardiomyopathy; and acquired cardiomyopathies such as myocarditis (e.g., viral myocarditis) and other inflammatory cardiomyopathies (e.g., Kawasaki disease and Chagas disease), stress (Tako-Tsubo) cardiomyopathy and peripartum cardiomyopathy. Representative secondary cardiomyopathies include: infiltrative cardiomyopathies associated with accumulation of abnormal substances between myocytes, non-limiting examples of which include cardiomyopathies resulting from amyloidosis, including primary, familial autosomal dominant, senile and secondary forms of amyloidosis, Gaucher disease, Hurler's disease and Hunter's disease; storage cardiomyopathies such a hemochromatosis, Fabry's disease, glycogen storage disease and Niemann-Pick disease; toxicity associated cardiomyopathies such as resulting from exposure to drugs, heavy metals and chemical agents, illustrative examples of which include such as chemotherapy cardiomyopathy (e.g., cardiomyopathies associated with exposure to: anthracyclines such as doxorubicin (Adriamycin) and daunorubicin; cyclophosphamide; and radiation) and alcoholic cardiomyopathy; endomyocardial cardiomyopathies including endomyocardial fibrosis, hypereosinophilic syndrome (Löeffler's endocarditis); granulomatous cardiomyopathies such as cardiac sarcoidosis; endocrine cardiomyopathies including those associated with diabetes mellitus, hyperthyroidism, hypothyroidism, hyperparathyroidism, pheochromocytoma and acromegaly; cardiofacial cardiomyopathies such as Noonan syndrome and lentiginosis; neuromuscular cardiomyopathies, which may be associated with accumulation of abnormal substances within myocytes, non-limiting examples of which include Friedreich's ataxia, Duchenne muscular dystrophy, Becker muscular dystrophy, Emery-Dreifuss muscular dystrophy, myotonic dystrophy, neurofibromatosis, and tuberous sclerosis; nutritional deficiency cardiomyopathies including cardiomyopathies associated with beriberi (thiamine), pallagra, scurvy, selenium, carnitine or kwashiorkor; autoimmune or collagen cardiomyopathies such as systemic lupus erythematosus, dermatomyositis, rheumatoid arthritis, scleroderma, and polyarteritis nodosa; and electrolyte imbalance cardiomyopathies.

13. The method of any one of embodiments 1 to 12, wherein the cardiomyopathy is ACM.

14. A method of treating or alleviating at least one symptom of ACM in a subject, the method comprising, consisting or consisting essentially of administering to the subject a CD14 antagonist antigen-binding molecule.

15. A method of reducing the number of premature ventricular contractions (PVCs) in a subject, the method comprising, consisting or consisting essentially of administering to the subject a CD14 antagonist antigen-binding molecule.

16. A method of treating or alleviating a symptom of atrial fibrillation in a subject, the method comprising, consisting or consisting essentially of administering to a subject having or at risk of developing atrial fibrillation a CD14 antagonist antigen-binding molecule.

17. A method of reducing the number of premature atrial contractions (PACs) in a subject, the method comprising, consisting or consisting essentially of administering to the subject a CD14 antagonist antigen-binding molecule.

18. The method of any one of embodiments 1 to 17, wherein the antigen-binding molecule is selected from:
(i) an antibody that comprises: a) an antibody VL domain, or antigen binding fragment thereof, comprising L-CDR1, L-CDR2 and L-CDR3, wherein: L-CDR1 comprises the sequence RASESVDSFGNSFMH [SEQ ID NO: 7] (3C10 L-CDR1); L-CDR2 comprises the sequence RAANLES [SEQ ID NO: 8] (3C10 L-CDR2); and L-CDR3 comprises the sequence QQSYEDPWT [SEQ ID NO: 9] (3C10 L-CDR3); and b) an antibody VH domain, or antigen binding fragment thereof, comprising H-CDR1, H-CDR2 and H-CDR3, wherein: H-CDR1 comprises the sequence SYAMS [SEQ ID NO: 10] (3C10 H-CDR1); H-CDR2 comprises the sequence SISSGGTTYYPDNVKG [SEQ ID NO: 11] (3C10 H-CDR2); and H-CDR3 comprises the sequence GYYDYHY [SEQ ID NO: 12] (3C10 H-CDR3);
(ii) an antibody that comprises: a) an antibody VL domain, or antigen binding fragment thereof, comprising L-CDR1, L-CDR2 and L-CDR3, wherein: L-CDR1 comprises the sequence RASESVDSYVNSFLH [SEQ ID NO: 13] (28C5 L-CDR1); L-CDR2 comprises the sequence RASNLQS [SEQ ID NO: 14] (28C5 L-CDR2); and L-CDR3 comprises the sequence QQSNEDPTT [SEQ ID NO: 15] (28C5 L-CDR3); and b) an antibody VH domain, or antigen binding fragment thereof, comprising H-CDR1, H-CDR2 and H-CDR3, wherein: H-CDR1 comprises the sequence SDSAWN [SEQ ID NO: 16] (28C5 H-CDR1); H-CDR2 comprises the sequence YISYSGSTSYNPSLKS [SEQ ID NO: 17] (28C5 H-CDR2); and H-CDR3 comprises the sequence GLRFAY [SEQ ID NO: 18] (28C5 H-CDR3);
(iii) an antibody that comprises: a) an antibody VL domain, or antigen binding fragment thereof, comprising L-CDR1, L-CDR2 and L-CDR3, wherein: L-CDR1 comprises the sequence RASESVDSYVNSFLH [SEQ ID NO: 13] (IC14 L-CDR1); L-CDR2 comprises the sequence RASNLQS [SEQ ID NO: 14] (IC14 L-CDR2); and L-CDR3 comprises the sequence QQSNEDPYT [SEQ ID NO: 27] (IC14 L-CDR3); and b) an antibody VH domain, or antigen binding fragment thereof, comprising H-CDR1, H-CDR2 and H-CDR3, wherein: H-CDR1 comprises the sequence SDSAWN [SEQ ID NO: 16] (IC14 H-CDR1); H-CDR2 comprises the sequence YISYSGSTSYNPSLKS [SEQ ID NO: 17] (IC14 H-CDR2); and H-CDR3 comprises the sequence GLRFAY [SEQ ID NO: 18] (IC14 H-CDR3);
(iv) an antibody that comprises: a) an antibody VL domain, or antigen binding fragment thereof, comprising L-CDR1, L-CDR2 and L-CDR3, wherein: L-CDR1 comprises the sequence RASQDIKNYLN [SEQ ID NO: 19] (18E12 L-CDR1); L-CDR2 comprises the sequence YTSRLHS [SEQ ID NO: 20] (18E12 L-CDR2); and L-CDR3 comprises the sequence QRGDTLPWT [SEQ ID NO: 21] (18E12 L-CDR3); and b) an antibody VH domain, or antigen binding fragment thereof, comprising H-CDR1, H-CDR2 and H-CDR3, wherein: H-CDR1 comprises the sequence NYDIS [SEQ ID NO: 22] (18E12 H-CDR1); H-CDR2 comprises the sequence VIWTSGGTNYNSAFMS [SEQ ID NO: 23] (18E12 H-CDR2); and H-CDR3 comprises the sequence GDGNFYLYNFDY [SEQ ID NO: 24] (18E12 H-CDR3);
(v) an antibody that comprises: a) an antibody VL domain, or antigen binding fragment thereof, comprising L-CDR1, L-CDR2 and L-CDR3, wherein: L-CDR1 comprises the sequence QNVGSNVDWY [SEQ ID NO: 34] (F1024-1-3 L-CDR1); L-CDR2 comprises the sequence KASNRY [SEQ ID NO: 35] (F1024-1-3 L-CDR2); and L-CDR3 comprises the sequence MQSNTNPPW [SEQ ID NO: 36] (F1024-1-3 L-CDR3); and b) an antibody VH domain, or antigen binding fragment thereof, comprising H-CDR1, H-CDR2 and H-CDR3, wherein: H-CDR1 comprises the sequence DYAMN [SEQ ID NO: 37] (F1024-1-3 H-CDR1); H-CDR2 comprises the sequence WINTQTGKPTYADDF [SEQ ID NO: 38] (F1024-1-3 H-CDR2); and H-CDR3 comprises the sequence TYFCTRSTFYYSSYIY [SEQ ID NO: 39] (F1024-1-3 H-CDR3);
(vi) an antibody that comprises: a) an antibody VL domain, or antigen binding fragment thereof, comprising L-CDR1, L-CDR2 and L-CDR3, wherein: L-CDR1 comprises the sequence KASQNVGSNVD [SEQ ID NO: 40] (F1024 L-CDR1); L-CDR2 comprises the sequence KASNRYT [SEQ ID NO: 41] (F1024 L-CDR2); and L-CDR3 comprises the sequence MQSNTNPPWT [SEQ ID NO: 42] (F1024 L-CDR3); and b) an antibody VH domain, or antigen binding fragment thereof, comprising H-CDR1, H-CDR2 and H-CDR3, wherein: H-CDR1 comprises the sequence DYAMN [SEQ ID NO: 37] (F1024 H-CDR1); H-CDR2 comprises the sequence WINTQTGKPTYADDFKQ [SEQ ID NO: 43] (F1024 H-CDR2); and H-CDR3 comprises the sequence STFYYSSYIYGWYFDF [SEQ ID NO: 44] (F1024 H-CDR3);
(vii) an antibody that comprises: a) an antibody VL domain, or antigen binding fragment thereof, comprising L-CDR1, L-CDR2 and L-CDR3, wherein: L-CDR1 comprises the sequence RASESVDSYGNSFMH [SEQ ID NO: 45] (r18D11 L-CDR1); L-CDR2 comprises the sequence LASNLES [SEQ ID NO: 46] (r18D11 L-CDR2); and L-CDR3 comprises the sequence QQNNGDPYT [SEQ ID NO: 47] (r18D11 L-CDR3); and b) an antibody VH domain, or antigen binding fragment thereof, comprising H-CDR1, H-CDR2 and H-CDR3, wherein: H-CDR1 comprises the sequence TYALN [SEQ ID NO: 48] (r18D11 H-CDR1); H-CDR2 comprises the sequence RIRSKSNNYTTYYADSVKD [SEQ ID NO: 49] (r18D11 H-CDR2); and H-CDR3 comprises the sequence PQSGTSFAY [SEQ ID NO: 50] (r18D11 H-CDR3); and
(viii) an antibody that comprises: a) an antibody VL domain, or antigen binding fragment thereof, comprising L-CDR1, L-CDR2 and L-CDR3, wherein: L-CDR1 comprises the sequence KASQYVGTNVA [SEQ ID NO: 51] (rMil2 L-CDR1); L-CDR2 comprises the sequence SASYRCS [SEQ ID NO: 52] (rMil2 L-CDR2); and L-CDR3 comprises the sequence QQYNTYVT [SEQ ID NO: 53] (rMil2 L-CDR3); and b) an antibody VH domain, or antigen binding fragment thereof, comprising H-CDR1, H-CDR2 and H-CDR3, wherein: H-CDR1 comprises the sequence TYWMN [SEQ ID NO: 54] (rMil2 H-CDR1); H-CDR2 comprises the sequence RIDPYDSETHYNONFKD [SEQ ID NO: 55] (rMil2 H-CDR2); and H-CDR3 comprises the sequence KEGROWGAYFDY [SEQ ID NO: 56] (rMil2 H-CDR3).

19. The method of any one of embodiments 1 to 18, wherein the antigen-binding molecule is selected from:
(i) an antibody comprising:
a VL domain that comprises, consists or consists essentially of the sequence:

[SEQ ID NO: 1]
QSPASLAVSLGQRATISCRASESVDSFGNSFMHWYQQKAGQPPKSSIYR
AANLESGIPARFSGSGSRTDFTMTINPVEADDVATYFCQQSYEDPWTFG
GGTKLGNQ (C310 VL);

and
a VH domain that comprises, consists or consists essentially of the sequence:

(3C10 VH)
[SEQ ID NO: 2]
LVKPGGSLKLSCVASGFTFSSYAMSWVRQTPEKRLEWVASISSGGTTYYPDNVKGRFTISRDNARNILY
LQMSSLRSEDTAMYYCARGYYDYHYWGQGTTLTVSS;

(ii) an antibody comprising:
a VL domain that comprises, consists or consists essentially of the sequence:

(28C5 VL)
[SEQ ID NO: 3]
QSPASLAVSLGQRATISCRASESVDSYVNSFLHWYQQKPGQPPKLLIYRASNLQS
GIPARFSGSGSRTDFTLTINPVEADDVATYCCQQSNEDPTTFGGGTKLEIK;

and
a VH domain that comprises, consists or consists essentially of the sequence:

(28C5 VH)
[SEQ ID NO: 4]
LQQSGPGLVKPSQSLSLTCTVTGYSITSDSAWNWIRQFPGNRLEWMGYISYSGSTSYNPSLKSRISITR
DTSKNQFFLQLNSVTTEDTATYYCVRGLRFAYWGQGTLVTVSA;

(iii) an antibody comprising:
a VL domain that comprises, consists or consists essentially of the sequence:

(18E12 VL)
[SEQ ID NO: 5]
QTPSSLSASLGDRVTISCRASQDIKNYLNWYQQPGGTVKVLIYYTSRLHSGVPSRFSGSGSGTDYSLTI
SNLEQEDFATYFCQRGDTLPWTFGGGTKLEIK;

and
a VH domain that comprises, consists or consists essentially of the sequence:

(18E12 VH)
[SEQ ID NO: 6]
LESGPGLVAPSQSLSITCTVSGFSLTNYDISWIRQPPGKGLEWLGVIWTSGGTNYNSAFMSRLSITKDN
SESQVFLKMNGLQTDDTGIYYCVRGDGNFYLYNFDYWGQGTTLTVSS;

(iv) an antibody comprising:
a VL domain that comprises, consists or consists essentially of the sequence:

(F1024-1-3 VL)
[SEQ ID NO: 57]
YIVMTQTPTSISISVGERVTMNCKASQNVGSNVDWYQQKTGQSPKLLIYKASNRYTGVPDRFTGSGSG
TDFTFTISNMQAVDLAVYYCMQSNTNPPWTFGGGTKLELKRA;

and
a VH domain that comprises, consists or consists essentially of the sequence:

(F1024-1-3 VH)
[SEQ ID NO: 58]
EVKLLESGGGLVQPSQTLSISCKASGYTFTDYAMNWVKQAPGDGLKWMGWINTQTGKPTYADDFKQ
RFVFSLETSASTAYLQINNLNIEDTATYFCTRSTFYYSSYIYGWYFDFWGPGTMVTVSS;

(v) an antibody comprising:
a VL domain that comprises, consists or consists essentially of the sequence:

(F1024 VL)
[SEQ ID NO: 59]
DIVMTQSPTSISISVGERVTMNCKASQNVGSNVDWYQQKTGQSPKLLIYKASNRYTGVPDRFTGSGS
GTDFTFTISNMQAVDLAVYYCMQSNTNPPWTFGGGTKLELKRA ;

and
a VH domain that comprises, consists or consists essentially of the sequence:

(F1024 VH)
[SEQ ID NO: 60]
QIQLVQSGPELKKPGESVKISCKASGYTFTDYAMNWVKQAPGNGLKWMGWINTQTGKPTYADDFKQ
RFVFSLETSASTAYLQINNLNIEDTATYFCTRSTFYYSSYIYGWYFDFWGPGTMVTVSS.

(vi) an antibody comprising:
a VL domain that comprises, consists or consists essentially of the sequence:

(r18D11 VL)
[SEQ ID NO: 61]
NIVLTQSPASLAVSLGQRATISCRASESVDSYGNSFMHWYQQKPGQPPKLLIYLASNLESGVPARFSGS
GSRTDFTLTIDPVEADDVATYYCQQNNGDPYTFGGGTKLEIIR;

and
a VH domain that comprises, consists or consists essentially of the sequence:

(r18D11 VH)
[SEQ ID NO: 62]
EVQLVESGGGLMQPKGSLKLSCAASGFTFKTYALNWVRQAPGTGLEWVARIRSKSNNYTTYYADSVK
DRFTISRDDSQNMLYLQMNNLKTEDTAMYYCVRPQSGTSFAYWGQGTLVTVSA;

and
(vii) an antibody comprising:
a VL domain that comprises, consists or consists essentially of the sequence:

(rMil2 VL)
[SEQ ID NO: 63]
DIVMTQSQKFMSTSVGDRVSVTCKASQYVGTNVAWYQQKPGQSPKALIQSASYRCSGVPDRFTGSG
SGTDFTLTISNVQSEDLADYFCQQYNTYVTFGGGTKLELKR;

and
a VH domain that comprises, consists or consists essentially of the sequence:

(rMil2 VH)
[SEQ ID NO: 64]
QVRLQQPGAELVRPGASVKLSCKASGYTFTTYWMNWVKQRPEDGLEWIGRIDPYDSETHYNQNFKDK
AILTVDKSSSTAYMQLSSLTYEDSAVYYCTRKEGRQWGAYFDYWGQGTTLTVSS.

20. The method according to any one of embodiments 1 to 19, wherein the antigen-binding molecule is humanized or chimeric.

21. The method of any one of embodiments 1 to 20, wherein the antigen-binding molecule comprises a light chain and a heavy chain, wherein:
the light chain comprises the amino acid sequence:

[SEQ ID NO: 25]
METDTILLWVLLLWVPGSTGDIVLTQSPASLAVSLGQRATISCRASESVDSYVNSFLHWYQQKP
GQPPKLLIYRASNLQSGIPARFSGSGSRTDFTLTINPVEADDVATYYCQQSNEDPYTFGGGTKLE
IKRTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC;

and
the heavy chain comprises the amino acid sequence:

[SEQ ID NO: 26]
MKVLSLLYLLTAIPGILSDVQLQQSGPGLVKPSQSLSLTCTVTGYSITSDSAWNWIRQFPGNRLE
WMGYISYSGSTSYNPSLKSRISITRDTSKNQFFLQLNSVTTEDTATYYCVRGLRFAYWGQGTLV
TVSSASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSVFLFPP
KPKDTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHY
TQKSLSLSLGK.

22. The method of any one of embodiments 1 to 21, wherein the antigen-binding molecule is the IC14 antibody.

23. The method of any one of embodiments 14 and 18 to 22, wherein the at least one symptom of ACM is selected from ventricular tachycardia, implantable cardioverter-defibrillator (ICD) shock, lung congestion, fluid retention, fatigue, heart murmurs, rapid heartbeat, arrhythmias, chest pain, light-headedness, fainting, dyspnea, peripheral edema, abdominal distention, myocardial fibro-fatty infiltration, embolization, syncope, angina, exercise intolerance, orthopnea, heart failure and sudden cardiac death (SCD).

24. The method of any one of embodiments 12 and 16 to 21, wherein the administration of the CD14 antagonist antigen-binding molecule improves one or more clinical parameters of the subject.

25. The method of embodiment 22, wherein the one or more clinical parameters are selected from reduced left ventricular ejection fraction, left ventricular hypertrophy, increased myocardial fibrosis, adverse cardiac remodeling, right ventricular dysfunction, aberrant heart geometry (e.g., increased right ventricular fractional area change), increased end-diastolic volume, increased frequency of premature ventricular contractions, and increased left ventricular mass.

26. The method of any one of embodiments 14 and 18 to 25, wherein the ACM is arrhythmogenic right ventricular cardiomyopathy (ARVC).

27. The method of any one of embodiments 16 and 18 to 22, wherein the symptom of atrial fibrillation is rapid heartbeat, arrhythmias, chest pain, light-headedness, fainting, dyspnea, heart failure, stroke or death.

28. The method of any one of embodiments 17 and 18 to 22, wherein the subject has atrial fibrillation (AF).

29. The method of any one of embodiments 1 to 28, wherein the subject has an implantable cardioverter defibrillator (ICD).

30. The method of embodiment 29, wherein the administration of a CD14 antagonist antigen-binding molecule reduces the number of electrical shocks delivered by the ICD to the subject.

31. The method of any one of embodiments 1 to 7 and 9 to 30, wherein the administration of a CD14 antagonist antigen-binding molecule improves a measure of ventricular function in the subject.

32. The method of embodiment 31, wherein the measure of ventricular function is ventricular strain, systolic function or diastolic function.

33. The method of embodiment 31 or embodiment 32, wherein the ventricular function is measured by echocardiography (echo), ambulatory rhythm (Holter) monitoring, cardiac CT scan, or cardiac magnetic resonance imaging.

34. The method of any one of embodiments 1 to 33, wherein the subject has one or more mutations in a gene associated with ACM.

35. The method of embodiment 34, wherein the gene associated with ACM is a gene encoding a component of the desmosome.

36. The method of embodiment 35, wherein the gene encodes Plakophilin-2 (PKP2), Desmoplakin (DSP), Desmoglein-2 (DSG2), Desmocollin-2 (DSC2), or Plakoglobin (JUP).

37. The method of embodiment 34, wherein the gene associated with ACM is Transmembrane protein 43 (TMEM43), Catenin Alpha 3 (CTNNA3), Desmin (DES), Lamin A/C (LMNA), Phospholamban (PLN), Ryanodine Receptor 2 (RYR2), Transforming growth factor beta-3 (TGFB3), Titin (TTN), Filamin C (FLNC), RNA Binding Motif Protein 20 (RBM20), Sodium Voltage-Gated Channel Alpha Subunit 5 (SCN5A), or BAG Cochaperone 3 (BAG3).

38. The method of any one of embodiments 1 to 37, wherein the subject is a human, a canine, a feline, an equine, a bovine, an ovine or a porcine.

39. The method of embodiment 38, wherein the subject is a human.

40. The method of any one of embodiments 1 to 39, wherein the CD14 antagonist antigen-binding molecule is administered systemically.

41. The method of any one of embodiments 1 to 39, wherein the CD14 antagonist antigen-binding molecule is administered locally to the heart (e.g., left ventricle) of the subject.

42. The method of any one of embodiments 1 to 41, wherein the CD14 antagonist antigen-binding molecule is administered at a daily dose of between about 0.1 mg/kg and 50 mg/kg (and all one tenth integer mg/kg units in between), or between about 0.2 mg/kg and 40 mg/kg (and all one tenth integer mg/kg units in between), or between about 0.5 mg/kg and 40 mg/kg (and all one tenth integer mg/kg units in between), or between about 1 mg/kg and 30 mg/kg (and all integer mg/kg units in between), or between about 2 mg/kg and 20 mg/kg (and all integer mg/kg units in between), or between about 4 mg/kg and 15 mg/kg (and all integer mg/kg units in between), or between about 5 mg/kg and 10 mg/kg (and all integer mg/kg units in between).

43. The method of embodiment 42, wherein the daily dose is administered in a single dose.

44. The method of embodiment 42, wherein the daily dose is administered in two doses.

45. The method of any one of embodiments 1 to 42, wherein the CD14 antagonist antigen-binding molecule is administered at a weekly dose of between about 1 mg/kg and 30 mg/kg (and all integer mg/kg units in between), or between about 2 mg/kg and 20 mg/kg (and all integer mg/kg units in between), or between about 4 mg/kg and 15 mg/kg (and all integer mg/kg units in between), or between about 5 mg/kg and 10 mg/kg (and all integer mg/kg units in between).

46. The method of any one of embodiments 1 to 45, wherein the CD14 antagonist antigen-binding molecule is administered to the subject over a period of about 1 day, or over a period of about 2 days, or over a period of about 3 days, or over a period of about 4 days, or over a period of about 5 days, or over a period of about 6 days, over a period of about 1 week, or over a period of about 2 weeks, or over a period of about 3 weeks, or over a period of about 4 weeks, or over a period of about 5 weeks, or over a period of about 6 weeks, or over a period of about 2 months, or over a period of about 3 months, or over a period of about 4 months, or over a period of about 5 months, or over a period of about 6 months, or over a period of about 7 months, or over a period of about 8 months, or over a period of about 9 months, or over a period of about 10 months, or over a period of about 11 months, or over a period of about 1 year, or over a period of about 14 months, or over a period of about 16 months, or over a period of about 18 months, or over a period of about 20 months, or over a period of about 22 months, or over a period of about 2 years.

47. The method of any one of embodiments 1 to 46, wherein the subject has not been diagnosed with autoimmune cardiomyopathy.

48. The method of any one of embodiments 1 to 47, wherein the subject has not undergone cardiopulmonary bypass surgery.

49. The method of any one of embodiments 1 to 48, wherein the subject is not an acute myocardial infarction patient.

50. The method of any one of embodiments 1 to 49, further comprising concurrently administering an ancillary cardiomyopathy therapeutic agent or intervention to the subject.

51. The method of embodiment 50, wherein the ancillary cardiomyopathy therapeutic agent is selected from angiotensin converting enzyme inhibitors (e.g., Enalipril, Lisinopril), angiotensin receptor blockers (e.g., Losartan, Valsartan), beta blockers (e.g., Lopressor, Toprol-XL), antiarrhythmic drugs (e.g., amiodarone (Cordarone, Pacerone), flecainide (Tambocor), ibutilide (Corvert) lidocaine (Xylocaine), procainamide (Procan, Procanbid), propafenone (Rythmol), quinidine, tocainide (Tonocarid)) digoxin, diuretics (e.g., Lasix; or Parkinson's disease therapies including, for example, levodopa, dopamine agonists (e.g., bromocriptine, pergolide, pramipexole, ropinirole, Piribedil, cabergoline, apomorphine, lisuride), statins (e.g., atorvastatin, fluvastatin, lovastatin, pitavastatin, pravastatin, rosuvastatin, simvastatin) MAO-B inhibitors (e.g., selegiline or rasagiline), amantadine, anticholinergics, antipsychotics (e.g., clozapine), cholinesterase inhibitors, modafinil, thiadiazolidindiones and non-steroidal anti-inflammatory drugs.

52. The method of any one of embodiments 1 to 51, further comprising performing a surgical procedure on the subject, wherein the surgical procedure is suitable for treating the cardiomyopathy.

53. The method of embodiment 52, wherein the surgical procedure comprises percutaneous coronary intervention (PCI; also known as coronary angioplasty), coronary artery bypass including coronary artery bypass grafting (CABG), implantation of a pacemaker, implantation of an implantable cardioverter-defibrillator (ICD), cardiac catheterization, revascularization and/or heart transplantation.

54. The method of embodiment 52 or embodiment 53, wherein the surgical procedure is performed before, at the same time as, or after administration of the CD14 antagonist antigen-binding molecule, optionally together with an ancillary cardiomyopathy therapeutic agent.

55. A CD14 antagonist antigen-binding molecule for use in treating or alleviating at least one symptom of a cardiomyopathy.

56. A CD14 antagonist antigen-binding molecule for use in alleviating or inhibiting development of adverse cardiac remodeling in a cardiomyopathy.

57. A CD14 antagonist antigen-binding molecule for use in improving cardiac function in a cardiomyopathy.

58. A CD14 antagonist antigen-binding molecule for use in improving ventricular function in a cardiomyopathy.

59. A CD14 antagonist antigen-binding molecule for use in improving atrial function in a cardiomyopathy.

60. A CD14 antagonist antigen-binding molecule for use in reducing the number of premature ventricular contractions (PVCs).

61. A CD14 antagonist antigen-binding molecule for use in treating or alleviating at least one symptom of atrial fibrillation.

62. A CD14 antagonist antigen-binding molecule for use or in reducing the number of premature atrial contractions (PACs).

63. Use of a CD14 antagonist antigen-binding molecule in the manufacture of a medicament for treating or alleviating at least one symptom of a cardiomyopathy.

64. Use of a CD14 antagonist antigen-binding molecule in the manufacture of a medicament for alleviating or inhibiting development of adverse cardiac remodeling in a cardiomyopathy.

65. Use of a CD14 antagonist antigen-binding molecule in the manufacture of a medicament for improving cardiac function in a cardiomyopathy.

66. Use of a CD14 antagonist antigen-binding molecule in the manufacture of a medicament for improving ventricular function in a cardiomyopathy.

67. Use of a CD14 antagonist antigen-binding molecule in the manufacture of a medicament for improving atrial function in a cardiomyopathy.

68. Use of a CD14 antagonist antigen-binding molecule in the manufacture of a medicament for reducing the number of premature ventricular contractions (PVCs).

69. Use of a CD14 antagonist antigen-binding molecule in the manufacture of a medicament for treating or alleviating at least one symptom of atrial fibrillation.

70. Use of a CD14 antagonist antigen-binding molecule in the manufacture of a medicament for reducing the number of premature atrial contractions (PACs).

71. The use of any one of embodiments 55 to 70, wherein the CD14 antagonist antigen-binding molecule is used in combination with one or more ancillary cardiomyopathy therapeutic agents or interventions.

In order that the disclosure may be readily understood and put into practical effect, particular preferred embodiments will now be described by way of the following non-limiting examples.

### EXAMPLES

### Example 1

Inhibiting Development of Arrhythmogenic Cardiomyopathy with an Anti-CD14 Antibody

Arrhythmogenic cardiomyopathy (ACM) is the name applied to a spectrum of non-ischemic heart muscle diseases with right-dominant (ARVC), left-dominant (ALVC) or biventricular patterns of myocardial injury and dysfunction. Most cases are caused by variants in genes that encode desmosomal proteins. In addition to forming cell-cell adhesion junctions, some desmosomal proteins participate in important signaling cascades including Wnt and Hippo pathways in which alterations have been implicated in the pathogenesis of ACM. However, exactly how variants in desmosomal genes lead to the complex clinical picture of arrhythmias and progressive myocardial injury remains largely unknown.

ACM is a leading cause of sudden death in young individuals and especially in athletes. As the name implies, it is associated with a high incidence of lethal ventricular arrhythmias. Indeed, the frequency of appropriate therapy for potentially lethal arrhythmias among all patients fitted with implantable cardiac defibrillators is higher for ACM patients than many other forms of heart disease. Fortunately, sudden deaths in young adults are infrequent, but they are obviously devastating to families in whom other members may also be at risk.

There are no drug therapies for ACM. The only treatment proven to reduce sudden death in ACM is an implantable cardioverter-defibrillator (ICD). While this can be lifesaving, it has major shortcomings including significant cost, associated psychological stress and quality-of-life issues. However, perhaps the most important limitation is that ICDs do nothing to treat the underlying heart muscle disease or prevent its progression. Even if the risk of sudden death can be reduced, chronic ongoing myocardial injury can progress to severe heart failure for which the only treatment is cardiac transplantation.

Considerable evidence now indicates that ACM is a chronic inflammatory disease. Inflammation in ACM has traditionally been considered in the context of inflammatory cell infiltrates in the heart, which are common in ACM patients. These inflammatory cells likely contribute to myocardial injury, but we also know that cardiac myocytes themselves mount an intense innate immune response in ACM and produce prodigious amounts of potent pro-inflammatory mediators. Activation of an innate immune response in cardiac myocytes occurs early in ACM and it appears to be a cell-autonomous process driven by upstream signals from ACM disease alleles. It is also a chronic, unremitting process.

Based on unpublished data showing that activation of an innate immune response in cardiac myocytes is a major mechanism promoting myocardial injury in ACM, and that disease progression is also mediated by actions of pro-inflammatory macrophages mobilized by signals from cardiac myocytes, the present inventors proposed testing an anti-CD14 antagonist antibody in a Dsg2^{mut/mut} mouse model of ACM to determine whether CD14 blockade would be effective in treating or slowing the development or progression of ACM.

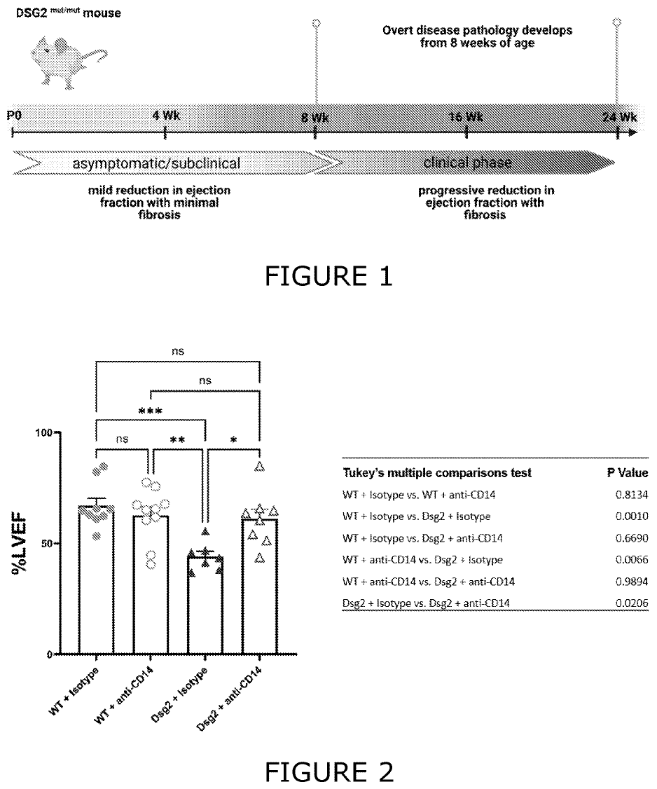

Dsg2^{mut/mut} mice show little if any apparent cardiac structural or functional derangements at 8 weeks of age, but during the ensuing 8 weeks, they develop a robust phenotype that recapitulates the most important clinical features seen in ACM patients, namely myocardial damage and arrhythmias, as illustrated in FIG. 1. This includes progressive deterioration of ventricular contractile function associated with development of extensive myocardial necrosis, fibrosis and inflammation. It also includes ECG abnormalities and arrhythmias. These structural and functional changes are associated with marked shifts in the distribution of various cardiac myocyte proteins including desmosomal proteins, connexins and ion channel proteins, proteins involved in the Wnt/β-catenin signaling pathway, and synapse associated protein 97 (SAP97), a chaperone protein involved in ion channel transport to intercalated disks. As disease progresses from 16 weeks onwards, there is progressive reduction in ejection fraction with significantly progression of myocardial fibrosis and inflammation.

Early Intervention with CD14 Antagonist Antibody

Starting at 8 weeks of age, Dsg2^{mut/mut} mice and wild-type (WT) control mice received intraperitoneal (i.p.) injections of anti-CD14 mAb or isotype control antibody at 14-day intervals over an 8-week period. The results shown in FIGS. 2-6 reveal that Dsg2^{mut/mut} mice at 16 weeks of age treated with isotype control exhibited a number of features representative of human ACM, including decreased left ventricular ejection fraction (LVEF), increased left ventricular mass (LVM) which is indicative of hypertrophic remodeling and decreased right ventricular fractional area change (RVFAC), an increase in premature ventricular contractions (PVCs) and an increase in fibrosis as measured by histologic analysis.

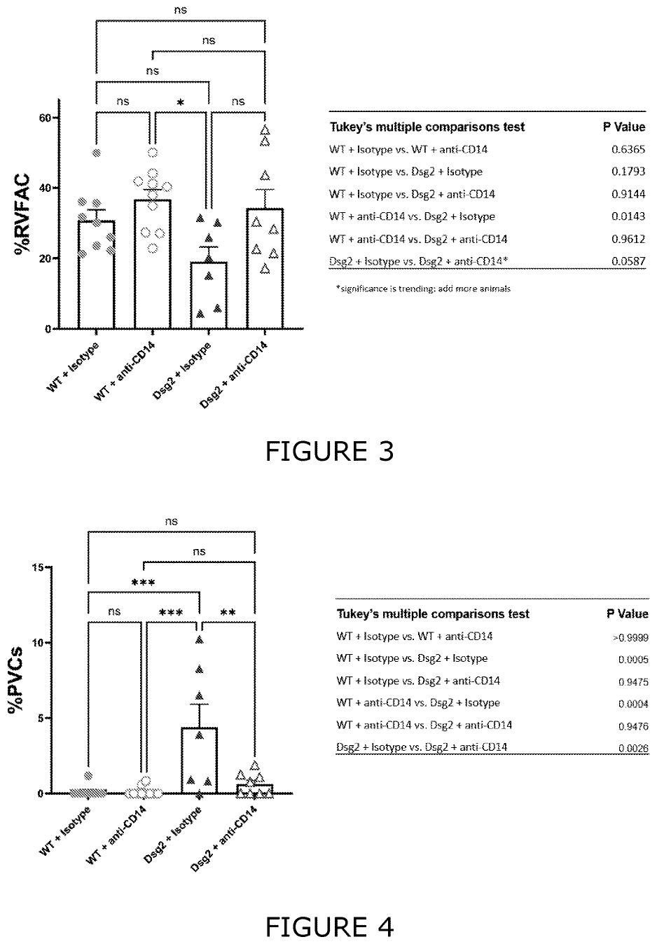

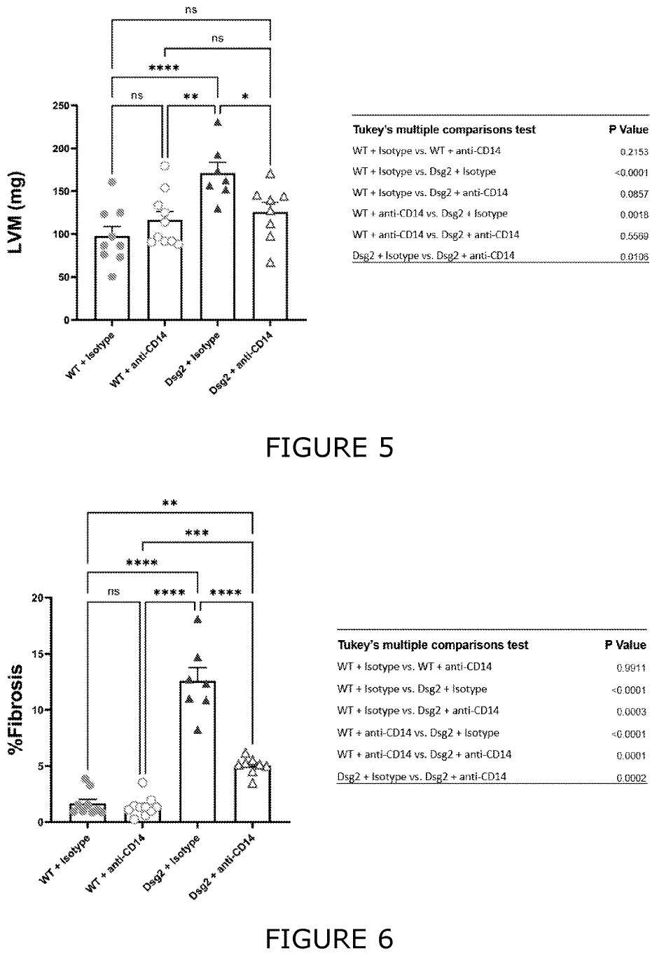

By contrast, anti-CD14 treatment of Dsg2^{mut/mut} mice was protective in all these pathologic changes. Notably:
Anti-CD14 treatment restored the % LVEF to a level seen in wild-type animals (FIG. 2).
Anti-CD14 treatment prevented right ventricular dysfunction by normalizing % RVFAC (FIG. 3).
Anti-CD14 treatment reduced ectopic heart beats by decreasing the frequency of PVCs, as measured by ECG (FIG. 4).
Anti-CD14 treatment restored the LVM to a level seen in wild-type animals, thereby minimizing hypertrophic remodeling (FIG. 5).
Anti-CD14 treatment reduced myocardial injury as measured by histologic analysis of fibrosis (FIG. 6).

Accordingly, anti-CD14 treatment inhibited development of ventricular dysfunction and adverse cardiac remodeling in the Dsg2^{mut/mut} model of arrhythmogenic cardiomyopathy.

These studies emphasize the important role played by CD14 in mediating disease progression in the mouse ACM model, with CD14 blockade maintaining ventricular function, decreasing the frequency of PVCs and reducing or blocking pathologic adverse cardiac remodeling. Given the underlying heart muscle disease and the ongoing disease progression which is seen in ACM patients fitted with an ICD treatment CD14 blockade may be efficacious, helping to mitigate the chronic ongoing myocardial injury that ultimately leads to heart failure and cardiac transplantation.

### Materials and Methods

### Mouse Model of ACM

The Jeffrey Saffitz lab at Beth Israel Deaconess Medical Center have a robust mouse model of ACM expressing a variant in the gene for the desmosomal protein desmoglein-2. Beginning at 8 weeks of age Dsg2^{mut/mut} mice begin to exhibit key features seen in ACM patients, including myocardial damage (necrotic/fibrotic lesions, myocyte apoptosis), contractile dysfunction, action potential remodeling and ventricular arrhythmias, and inflammation (inflammatory infiltrates, production of inflammatory cytokines), with NFκB activated in cardiac myocytes.

### Experimental Design

Starting at 8 weeks of age Dsg2^{mut/mut} mice and WT control mice (n=7-10/group) received intraperitoneal (i.p.) injections of anti-CD14 mAb (biG53 LALA-PG) or isotype control antibody. Mice were injected every 14 days (100 μL of anti-CD14 or isotype) at a dose of 5 mg/kg.

### Readouts

Mice were echoed at 8 weeks of age, prior to the initiation of treatment and at end of study. Electrocardiograms (ECGs) were performed at 16 weeks of age (8 weeks of dosing), after which mice were sacrificed. ECGs were assessed for premature ventricular contractions (PVCs) and runs of ventricular tachycardia; echo parameters included ejection fraction and measurements of ventricular walls/chambers. Upon sacrifice, hearts were isolated, with half fixed in formalin for histological analysis of necrosis and fibrosis and the other half frozen for subsequent analyses.

### Example 2

Treatment of Arrhythmogenic Cardiomyopathy with an Anti-CD14 Antibody

The present inventors also tested whether an anti-CD14 antagonist antibody in the Dsg2^{mut/mut} mouse model would be effective in treating or slowing the progression of ACM and promote recovery of contractile dysfunction in animals with established disease.

Late Intervention with CD14 Antagonist Antibody

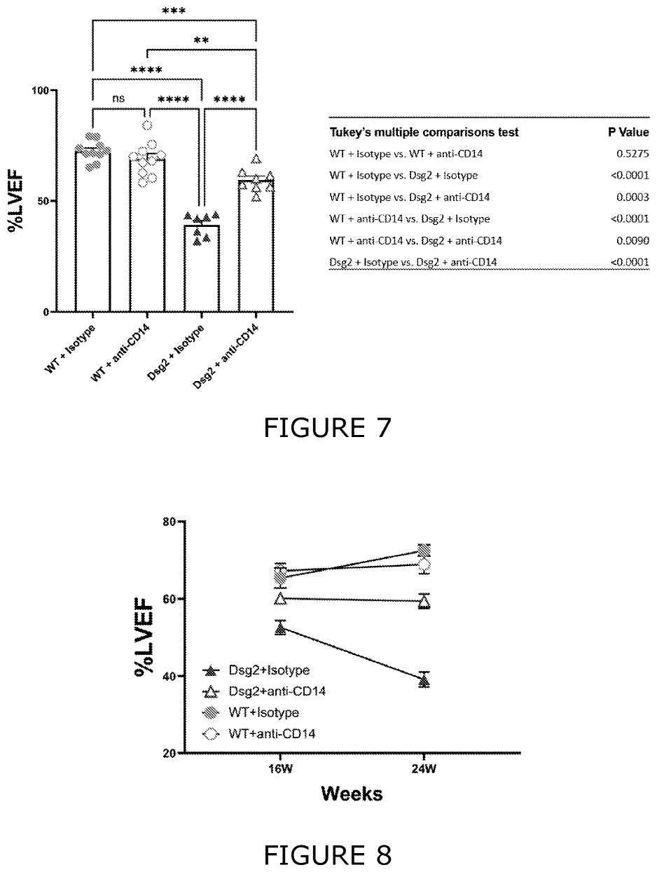

Starting at 16 weeks of age, Dsg2^{mut/mut} mice and WT control mice received i.p. injections of anti-CD14 mAb or isotype control antibody at 14-day intervals over an 8-week period. The results shown in FIGS. 7-9 reveal that Dsg2^{mut/mut} mice at 24 weeks of age treated with isotype control exhibited markedly worse disease symptoms, including significantly lower % LVEF and markedly higher fibrosis, than before the isotype control antibody was administered to them at 16 weeks of age.

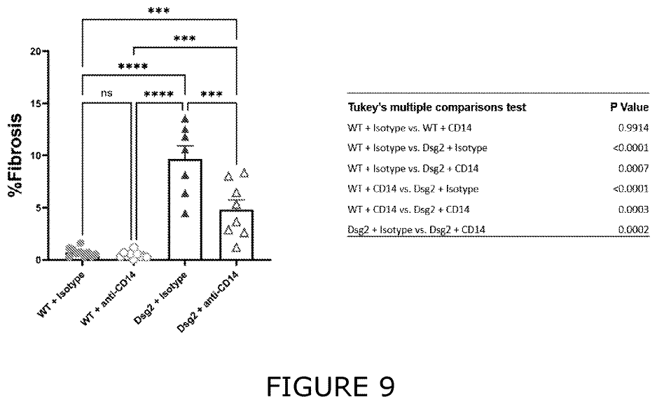

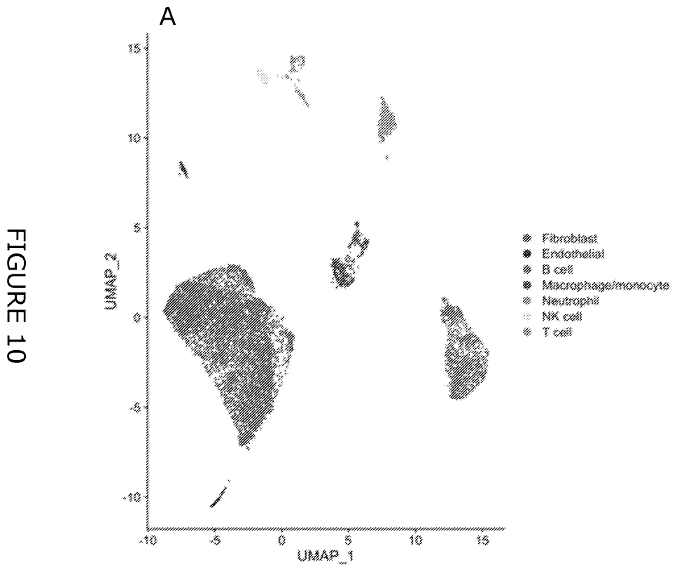

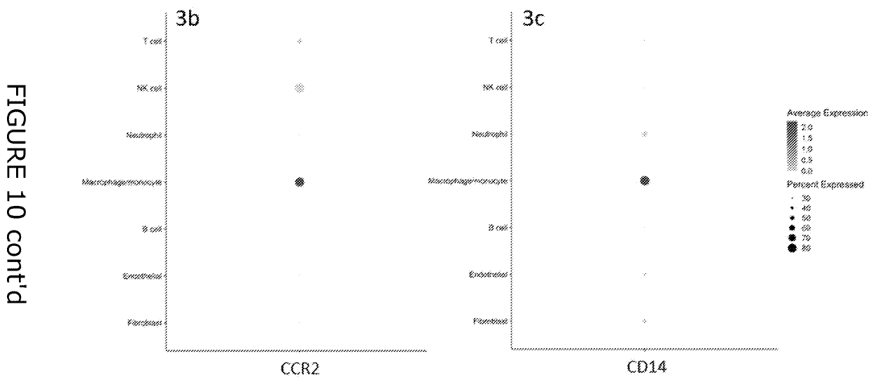

On the other hand, anti-CD14 treatment of Dsg2^{mut/mut} mice at 16 weeks of age noticeably impeded/slowing further disease progression, including mitigating against further deterioration of % LVEF (FIGS. 7, 8) and myocardial fibrosis (FIG. 9).

Accordingly, anti-CD14 treatment in animals with established disease stabilized the cardiomyopathy by impeding or markedly slowing down further worsening of ventricular dysfunction and adverse cardiac remodeling.

### Example 3

Inflammatory Macrophages from Human and Murine Subjects with Cardiomyopathies Co-Express CCR2 and CD14

It is known that the mouse heart contains a heterogeneous population of functionally distinct macrophages with remarkable effects on cardiovascular disease pathogenesis. Notably, CCR2− and CCR2+ macrophages are known to have distinct functions in the heart. In this regard, CCR2-macrophages are involved in various forms of tissue remodeling such as coronary development, postnatal coronary growth, and cardiac regeneration (Lavine et al., Proc. Natl Acad. Sci. USA 2014; 111, 16029-16034; Leid et al., Circ. Res. 2016; 118, 1498-1511). For example, following neonatal cardiomyocyte injury, CCR2-macrophages orchestrate cardiac tissue regeneration and functional recovery of the heart through expansion of the coronary vasculature, cardiomyocyte proliferation, and physiological cardiomyocyte hypertrophy. In the absence of CCR2− macrophages, the pediatric mouse heart demonstrates little regenerative capacity. Within the resting adult heart, CCR2+ macrophages are a particularly inflammatory population derived from circulating Ly6C^{high}CCR2+ blood monocytes that infiltrate heart following various forms of tissue injury.

It is also known that CCR2+ and CCR2− macrophages from human patients with dilated cardiomyopathy (DCM) and ischemic cardiomyopathy (ICM) uniformly express CD14 and that CCR2+ macrophages represent an inflammatory population whose abundance is associated with persistent left ventricular (LV) systolic dysfunction and adverse remodeling (Bajpai et al., Nat Med. 2018; 24 (8): 1234-1245).

Based on this knowledge and the results disclosed in Examples 1 and 2, the present inventors hypothesized that the hearts of Dsg2^{mut/mut} mice would also contain pro-inflammatory macrophage populations that co-express CCR2 and CD14. To test this hypothesis, they performed single-cell RNA sequence (scRNA seq) analysis of cardiac tissue obtained from 12-week-Dsg2^{mut/mut} mice according to methods described by Chelko, S. P. et al. (Mechanisms of Innate Immune Injury in Arrhythmogenic Cardiomyopathy. bioRxiv Prepr. Serv. Biol. (2023) doi: 10.1101/2023.07.12.548682). This analysis identified 7 distinct stromal and immune cell types (FIG. 10A), including fibroblasts, endothelial cells, B cells, monocytes/macrophages, neutrophils, NK cells and T cells. Within the monocyte/macrophage cluster, a significant macrophage subpopulation was identified co-expressing CCR2 (FIG. 10B) and CD14 (FIG. 10C). These results show that Dsg2^{mut/mut} mice contain pro-inflammatory CCR2+CD14+ monocytes and macrophages that are likely associated with the development of cardiomyopathy disease in the Dsg2^{mut/mut} model of ACM.

Taken together with the knowledge that pro-inflammatory CCR2+CD14+ macrophages are also present in other cardiomyopathies, including DCM and ICM, the data presented herein indicate that CCR2+CD14+ cardiac macrophages are likely associated with the development of cardiomyopathies in general, and that targeting these macrophages with anti-CD14 antagonists is expected to be beneficial for alleviating or inhibiting development of any one or more of reduced left ventricular ejection fraction, left ventricular hypertrophy, increased myocardial fibrosis, adverse cardiac remodeling, right ventricular dysfunction, aberrant heart geometry (e.g., increased right ventricular fractional area change), increased end-diastolic volume, increased frequency of premature ventricular contractions, and increased left ventricular mass, reduced cardiac function, including reduced ventricular function and reduced atrial function, in cardiomyopathie subjects or subjects at risk of developing cardiomyopathies.

### Example 4

Pet Imaging of ⁶⁸Ga-DOTA Tetraazacyclododecane Tetraacetic Acid-ECL1i Extracellular Loop 1 Inverso Imaging in a DSG2^{mut/mut} Mouse Model of Arrhythmogenic Cardiomyopathy

The C—C chemokine receptor type 2 (CCR2) is specifically expressed on inflammatory populations of monocytes and macrophages that contribute to the development and progression of heart failure and cardiac dysfunction post cardiac injury. The present inventors have disclosed herein preservation of cardiac function, reduction of fibrosis and reduction of pre ventricular contractions in Dsg2^{mut/mut} mice by treatment with big53 LALA PG anti-CD14 mAb. Left untreated these mice develop a pathology in line with arrhythmogenic cardiomyopathy (ACM) patients over the course of 8 weeks of age. Furthermore, using single cell RNA Seq technology, it was identified that treatment with anti-CD14 specifically altered the genetic expression profile of CCR2+ macrophages in these mice and it was reasoned that this mechanism of action is related to the therapeutic benefits that were observed in those mice.

In the present study, a peptide-based imaging probe ⁶⁸Ga-DOTA tetraazacyclododecane tetraacetic acid-ECL1i extracellular loop 1 inverso (68Ga-DOTA-ECL1i) was evaluated, which specifically recognizes the CCR2+ monocytes and macrophages that are known to contribute to fibrosis and adverse remodeling in cardiac and pulmonary injuries (Lavine et al., Nat. Cardiovasc Res. https://doi.org/10.1038/s44161-023-00335-6; Liu et al., Radiology 2017; 283 (3): 758-768). The purpose of the study was to non-invasively detect cardiac inflammation in a mouse model of ACM utilizing the ⁶⁸Ga-DOTA-ECL1i probe to investigate if treatment with anti-CD14 reduced the CCR2+ signature in treated versus control animals, and further validate the proposed mechanism of action that CD14 alters the activity and recruitment of CCR2+ macrophages in the hearts of ACM mice.

### Materials and Methods

### Active Ingredient Dosing

Dsg2^{mut/mut} mice and wild type C57black mice were treated weekly for 4 weeks from 8 weeks of age with either big53 LALA PG anti-CD14 mAb (5 mg/kg), or IgG2A LALAPG isotype control (5 mg/kg).

### Imaging

Dynamic PET image acquisition (from 0 to 60 min) and corresponding computed tomographic (CT) images were obtained by using cross-calibrated scanners (Inveon microPET/CT [Siemens, Malvern, Pa] or Focus 220 PET [Concorde Microsystems, Knoxville, Tenn]) immediately after tail vein injection of ⁶⁸Ga-DOTA-ECL1i (3.7 MBq per mouse). Organ uptake was calculated as the percentage injected dose (% ID) per gram of tissue in three-dimensional regions of interest from PET images, without correction for partial volume effect, by using software (Inveon Research Workplace, Siemens).

### Statistical Methods:

Data were analyzed by using software (Prism, version 6.07; GraphPad, La Jolla, Calif.). Multiple means were compared by using a one- or two-way analysis of variance with the Tukey test. P<0.05 was indicative of a statistically significant difference.

### Results

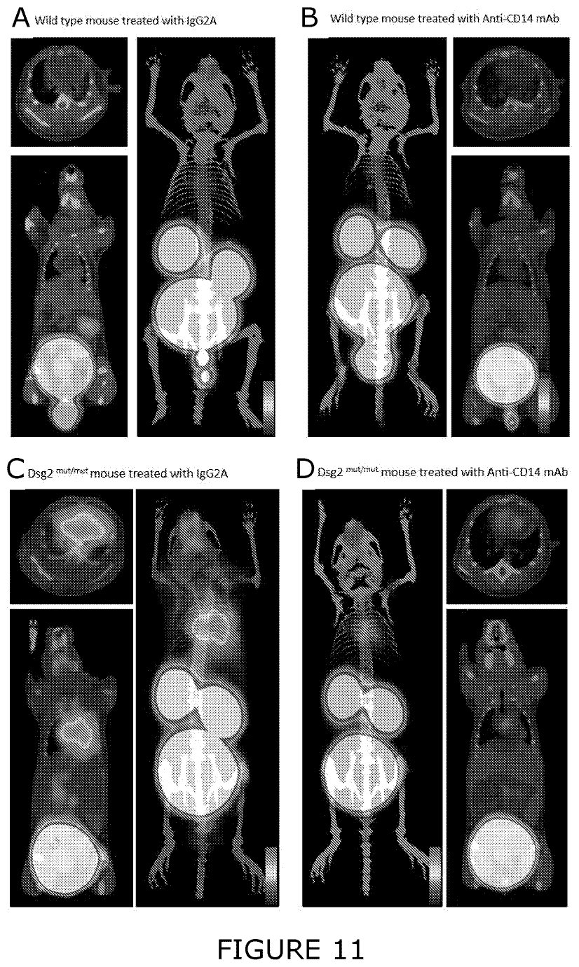

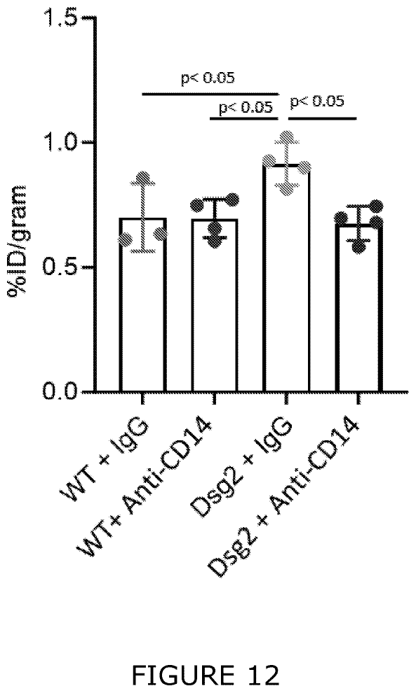

⁶⁸Ga-DOTA-ECL1i uptake in the hearts of wild type mice treated with either anti-CD14 or isotype control were equivalent (FIGS. 11A and 11B). In comparison the uptake of the PET probe was much higher in the Dsg2^{mut/mut} mice treated with the IgG2A LALAPG isotype control (FIG. 11B) and that this was reduced in anti-CD14 treated animals to levels equivalent to wild type mice (FIG. 11D). The uptake difference was further validated by calculation of the percentage injected dose per gram of tissue of the heart showing statistical significance (p<0.05) between the untreated and treated Dsg2^{mut/mut} mice (FIG. 12).

### Conclusion

Imaging of Dsg2^{mut/mut} mice injected with of 68Ga-DOTA-ECL1i PET probe confirmed that CCR2+ macrophages, which have been established by previous studies to uniformly express CD14, are localized in the heart of the ACM mouse at the 14-week time point, noting that disease pathology in these mice is overtly established at 8 weeks of age. Furthermore, treatment of the mice with an anti-CD14 antibody significantly (p<0.05) reduced the CCR2+ signature in the diseased mice. This study highlights that there is a marked reduction in CCR2+CD14+ macrophages in the hearts of treated ACM animals, which coincides with restoration of left ventricular ejection fraction (% LVEF) to a level seen in wild-type animals, as shown in FIG. 2, inhibition of right ventricular dysfunction by normalizing right ventricular fractional area change (% RVFAC), as shown in FIG. 3, reduction of ectopic heart beats by decreasing the frequency of premature ventricular contractions (PVCs), as shown in FIG. 4, restoration of left ventricular mass (LVM) to a level seen in wild-type animals with associated decrease or minimization of hypertrophic remodeling, as shown in FIG. 5, and reduction of myocardial fibrosis, as shown in FIG. 6, which, in turn, reduces myocardial injury.

### Example 5

### Myocardium of Human Cardiac Sarcoidosis Patient Contains CCR2+ Monocytes and Macrophages

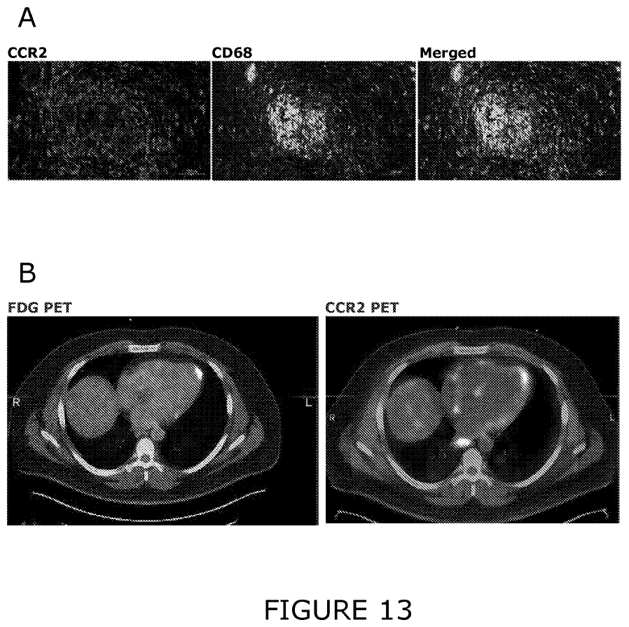

To assess whether the myocardium of human cardiac sarcoidosis patients also contains pro-inflammatory CCR2+ monocytes and macrophages, immunostaining was performed on LV tissue obtained from a patient with cardiac sarcoidosis (CS). Dual immunostaining for CD68 (pan-macrophage marker) and CCR2 (FIG. 13A) revealed that CCR2+ monocytes and macrophages are present within the periphery of the granuloma surrounding the multinucleated giant cells and that CCR2+ cell abundance correlated with markers of disease activity: particularly, macrophage proliferation and pS6 kinase staining (reporter of mTOR activation).

The presence of pro-inflammatory CCR2+ monocytes and macrophages was further investigated by PET imaging of myocardial tissue obtained from a patient with active CS and presenting with palpitations and high degree atrioventricular (AV) conduction block. In this study, the CS patient was subjected to serial PET imaging of ¹⁸F-FDG and ⁶⁸Ga-DOTA-ECL1i to assess uptake of these radiotracers by the patient's myocardium. ¹⁸F-FDG is used routinely for assessing viability of the myocardium and for detecting infarct areas including is CS and other inflammatory cardiomyopathies (Khalaf et al., Methodist Debakey Cardiovasc J. 2020; 16 (2): 122-129). ⁶⁸Ga-DOTA-ECL1i contains a 7 amino acid peptide that allosterically binds CCR2 and uptake of this radiotracer by myocardial tissue is used to image CCR2+ monocyte and CCR2+ macrophage infiltration into the heart. Notably, ⁶⁸Ga-DOTA-ECL1i specifically binds human heart failure specimens, with signal intensity associated with CCR2+ macrophage abundance Heo et al., J Nucl Med. 2021; 62 (1): 111-114).

The results of the PET imaging (FIG. 13B) of the CS patient reveal that FDG uptake was present in the apex of the LV, indicating presence of an infarct, and that CCR2 uptake was present within the apex, septum, and basal lateral wall of the LV, as well as in a parabrachial lymph node, indicating development and/or progression of cardiomyopathy disease.

Since it is established that pro-inflammatory cardiac CCR2+ macrophages from cardiomyopathy patients uniformly express CD14, the present inventors propose, in the context of the present disclosure, that pro-inflammatory cardiac CCR2+CD14+ macrophages are also present in CS patients and that targeting these macrophages with anti-CD14 antagonists is expected to be beneficial for treating or inhibiting the development of CS and associated clinical parameters (e.g., reduced left ventricular ejection fraction, left ventricular hypertrophy, increased myocardial fibrosis, adverse cardiac remodeling, right ventricular dysfunction, aberrant heart geometry (e.g., increased right ventricular fractional area change), increased end-diastolic volume, increased frequency of premature ventricular contractions, and increased left ventricular mass).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

What is claimed is:

1. A method of treating or alleviating at least one symptom of a cardiomyopathy in a subject, the method comprising, consisting or consisting essentially of administering to the subject a CD14 antagonist antigen-binding molecule.

2. The method of claim 1, wherein the at least one symptom is selected from ventricular tachycardia, implantable cardioverter-defibrillator (ICD) shock, lung congestion, fluid retention, fatigue, heart murmurs, rapid heartbeat, arrhythmias, chest pain, light-headedness, fainting, dyspnea, peripheral edema, abdominal distention, myocardial fibro-fatty infiltration, embolization, syncope, angina, exercise intolerance, orthopnea, heart failure and sudden cardiac death (SCD).

3. The method of claim 1 or claim 2, wherein the administration of the CD14 antagonist antigen-binding molecule improves one or more clinical parameters of the subject.

4. The method of claim 3, wherein the one or more clinical parameters are selected from reduced left ventricular ejection fraction, left ventricular hypertrophy, increased myocardial fibrosis, adverse cardiac remodeling, right ventricular dysfunction, aberrant heart geometry (e.g., increased right ventricular fractional area change), increased end-diastolic volume, increased frequency of premature ventricular contractions, and increased left ventricular mass.

5. A method of alleviating or inhibiting development of adverse cardiac remodeling in a subject with a cardiomyopathy, the method comprising, consisting or consisting essentially of administering to the subject a CD14 antagonist antigen-binding molecule.

6. A method of improving cardiac function in a subject with a cardiomyopathy, the method comprising, consisting or consisting essentially of administering to the subject a CD14 antagonist antigen-binding molecule.

7. The method of claim 6, wherein improving cardiac function comprises improving left ventricular function, improving fractional shortening, improving ejection fraction, reducing end-diastolic volume, decreasing left ventricular mass, decreasing arrhythmias, reducing frequency of heart murmurs, reducing heart rate, normalizing of heart geometry, or a combination thereof.

8. A method of improving ventricular function in a subject with a cardiomyopathy, the method comprising, consisting or consisting essentially of administering to the subject a CD14 antagonist antigen-binding molecule.

9. A method of improving atrial function in a subject with a cardiomyopathy, the method comprising, consisting or consisting essentially of administering to the subject a CD14 antagonist antigen-binding molecule.

10. The method of any one of claims 1 to 9, wherein the cardiomyopathy is a primary cardiomyopathy or a secondary cardiomyopathy.

11. The method of claim 10, wherein the primary cardiomyopathy is selected from genetic cardiomyopathies (e.g., hypertrophic cardiomyopathy, arrhythmogenic cardiomyopathy (ACM), left ventricular non-compaction, conduction system disease, ion channelopathy (e.g., long-QT syndrome), Brugada syndrome, catecholaminergic polymorphic ventricular tachycardia, short-QT syndrome and idiopathic ventricular fibrillation), mixed (genetic and non-genetic) cardiomyopathies (e.g., dilated cardiomyopathy and restrictive cardiomyopathy), and acquired cardiomyopathies (e.g., myocarditis such as viral myocarditis and other inflammatory cardiomyopathies such as Kawasaki disease and Chagas disease, stress (Tako-Tsubo) cardiomyopathy and peripartum cardiomyopathy).

12. The method of claim 10, wherein the secondary cardiomyopathy is selected from genetic cardiomyopathies, illustrative examples of which include hypertrophic cardiomyopathy, arrhythmogenic cardiomyopathy (ACM), left ventricular non-compaction, conduction system disease, ion channelopathy (e.g., long-QT syndrome), Brugada syndrome, catecholaminergic polymorphic ventricular tachycardia, short-QT syndrome and idiopathic ventricular fibrillation); mixed (genetic and non-genetic) cardiomyopathies, non-limiting examples of which include dilated cardiomyopathy and restrictive cardiomyopathy; and acquired cardiomyopathies such as myocarditis (e.g., viral myocarditis) and other inflammatory cardiomyopathies (e.g., Kawasaki disease and Chagas disease), stress (Tako-Tsubo) cardiomyopathy and peripartum cardiomyopathy. Representative secondary cardiomyopathies include: infiltrative cardiomyopathies associated with accumulation of abnormal substances between myocytes, non-limiting examples of which include cardiomyopathies resulting from amyloidosis, including primary, familial autosomal dominant, senile and secondary forms of amyloidosis, Gaucher disease, Hurler's disease and Hunter's disease; storage cardiomyopathies such a hemochromatosis, Fabry's disease, glycogen storage disease and Niemann-Pick disease; toxicity associated cardiomyopathies such as resulting from exposure to drugs, heavy metals and chemical agents, illustrative examples of which include such as chemotherapy cardiomyopathy (e.g., cardiomyopathies associated with exposure to: anthracyclines such as doxorubicin (Adriamycin) and daunorubicin; cyclophosphamide; and radiation) and alcoholic cardiomyopathy; endomyocardial cardiomyopathies including endomyocardial fibrosis, hypereosinophilic syndrome (Löeffler's endocarditis); granulomatous cardiomyopathies such as cardiac sarcoidosis; endocrine cardiomyopathies including those associated with diabetes mellitus, hyperthyroidism, hypothyroidism, hyperparathyroidism, pheochromocytoma and acromegaly; cardiofacial cardiomyopathies such as Noonan syndrome and lentiginosis; neuromuscular cardiomyopathies, which may be associated with accumulation of abnormal substances within myocytes, non-limiting examples of which include Friedreich's ataxia, Duchenne muscular dystrophy, Becker muscular dystrophy, Emery-Dreifuss muscular dystrophy, myotonic dystrophy, neurofibromatosis, and tuberous sclerosis; nutritional deficiency cardiomyopathies including cardiomyopathies associated with beriberi (thiamine), pallagra, scurvy, selenium, carnitine or kwashiorkor; autoimmune or collagen cardiomyopathies such as systemic lupus erythematosus, dermatomyositis, rheumatoid arthritis, scleroderma, and polyarteritis nodosa; and electrolyte imbalance cardiomyopathies.

13. The method of any one of claims 1 to 12, wherein the cardiomyopathy is ACM.

14. The method of any one of claims 1 to 12, wherein the cardiomyopathy is a non-ACM cardiomyopathy.

15. The method of any one of claims 1 to 12 and 14, wherein the cardiomyopathy is cardiac sarcoidosis.

16. A method of treating or alleviating at least one symptom of ACM in a subject, the method comprising, consisting or consisting essentially of administering to the subject a CD14 antagonist antigen-binding molecule.

17. A method of reducing the number of premature ventricular contractions (PVCs) in a subject, the method comprising, consisting or consisting essentially of administering to the subject a CD14 antagonist antigen-binding molecule.

18. A method of treating or alleviating a symptom of atrial fibrillation in a subject, the method comprising, consisting or consisting essentially of administering to a subject having or at risk of developing atrial fibrillation a CD14 antagonist antigen-binding molecule.

19. A method of reducing the number of premature atrial contractions (PACs) in a subject, the method comprising, consisting or consisting essentially of administering to the subject a CD14 antagonist antigen-binding molecule.

20. The method of any one of claims 1 to 19, further comprising concurrently administering with the CD14 antagonist antigen-binding molecule at least one ancillary therapeutic agent or intervention for treating or inhibiting the development of a cardiomyopathy.
